SEQUENCE LISTING (524 sequences)

SEQ ID NO: 1 - protein, 24 aa, Artificial Sequence
  Synthetic
  MISC_FEATURE (1)..(1): Xaa = 6-Aminohexanoic acid
XRTRRRRRRQRVRIAYEEIFVRNM

SEQ ID NO: 2 - protein, 22 aa, Artificial Sequence
  Synthetic
TKRKRKKQRVKIAYEEIFVKNM

SEQ ID NO: 3 - protein, 21 aa, Artificial Sequence
  Synthetic
KRKRKKQRVKIAYEEIFVKNM

SEQ ID NO: 4 - protein, 20 aa, Artificial Sequence
  Synthetic
RKRKKQRVKIAYEEIFVKNM

SEQ ID NO: 5 - protein, 23 aa, Artificial Sequence
  Synthetic
KTKRKRKKQRVKIAYEEIFVKNM

SEQ ID NO: 6 - protein, 23 aa, Artificial Sequence
  Synthetic
KTKRKRKKQRVKIAYEEVFVKNM

SEQ ID NO: 7 - protein, 24 aa, Artificial Sequence
  Synthetic
KNKSKGVVKIQRRKAPFVVYESIN

SEQ ID NO: 8 - protein, 24 aa, Artificial Sequence
  Synthetic
KKSSKRAKTQRRRKSFVKMYENIH

SEQ ID NO: 9 - protein, 24 aa, Artificial Sequence
  Synthetic
KKSSKRAKTQRRRKSFVKMYENIH

SEQ ID NO: 10 - protein, 19 aa, Artificial Sequence
  Synthetic
KRKKQRVKIAYEEIFVKNM

SEQ ID NO: 11 - protein, 18 aa, Artificial Sequence
  Synthetic
RKKQRVKIAYEEIFVKNM

SEQ ID NO: 12 - protein, 17 aa, Artificial Sequence
  Synthetic
KKQRVKIAYEEIFVKNM

SEQ ID NO: 13 - protein, 22 aa, Artificial Sequence
  Synthetic
KTKRKRKKQRVKIAYEEIFVKN

SEQ ID NO: 14 - protein, 21 aa, Artificial Sequence
  Synthetic
KTKRKRKKQRVKIAYEEIFVK

SEQ ID NO: 15 - protein, 20 aa, Artificial Sequence
  Synthetic
KTKRKRKKQRVKIAYEEIFV

SEQ ID NO: 16 - protein, 19 aa, Artificial Sequence
  Synthetic
KTKRKRKKQRVKIAYEEIF

SEQ ID NO: 17 - protein, 18 aa, Artificial Sequence
  Synthetic
KTKRKRKKQRVKIAYEEI

SEQ ID NO: 18 - protein, 17 aa, Artificial Sequence
  Synthetic
KTKRKRKKQRVKIAYEE

SEQ ID NO: 19 - protein, 22 aa, Artificial Sequence
  Synthetic
TKRKRKKQRVKIAYEEIFVQNM

SEQ ID NO: 20 - protein, 21 aa, Artificial Sequence
  Synthetic
KRKRKKQRVKIAYEEIFVQNM

SEQ ID NO: 21 - protein, 20 aa, Artificial Sequence
  Synthetic
RKRKKQRVKIAYEEIFVQNM

SEQ ID NO: 22 - protein, 19 aa, Artificial Sequence
  Synthetic
KRKKQRVKIAYEEIFVQNM

SEQ ID NO: 23 - protein, 18 aa, Artificial Sequence
  Synthetic
RKKQRVKIAYEEIFVQNM

SEQ ID NO: 24 - protein, 17 aa, Artificial Sequence
  Synthetic
KKQRVKIAYEEIFVQNM

SEQ ID NO: 25 - protein, 22 aa, Artificial Sequence
  Synthetic
KTKRKRKKQRVKIAYEEIFVQN

SEQ ID NO: 26 - protein, 21 aa, Artificial Sequence
  Synthetic
KTKRKRKKQRVKIAYEEIFVQ

SEQ ID NO: 27 - protein, 25 aa, Artificial Sequence
  Synthetic
  MISC_FEATURE (1)..(1): Xaa = Acylated Arginine
  MISC_FEATURE (24)..(24): Xaa = 6-Aminohexanoic acid
XTRRRRRRQRVRIAYEEIFVRNMXK

SEQ ID NO: 28 - protein, 25 aa, Artificial Sequence
  Synthetic
  MISC_FEATURE (1)..(1): Xaa = Acylated Arginine
  MISC_FEATURE (24)..(24): Xaa = 6-Aminohexanoic acid
  MISC_FEATURE (25)..(25): The side chain of Lysine at position 24 is covalently bound to an Ida linker forming a homodimer of residues 1 - 25.
XTRRRRRRQRVRIAYEEIFVRNMXK

SEQ ID NO: 29 - protein, 19 aa, Artificial Sequence
  Synthetic
KTKRKRKKQAYEEIFVQNM

SEQ ID NO: 30 - protein, 19 aa, Artificial Sequence
  Synthetic
KTKRKRKKQRYEEIFVQNM

SEQ ID NO: 31 - protein, 19 aa, Artificial Sequence
  Synthetic
KTKRKRKKQRVEEIFVQNM

SEQ ID NO: 32 - protein, 19 aa, Artificial Sequence
  Synthetic
KTKRKRKKQRVKEIFVQNM

SEQ ID NO: 33 - protein, 19 aa, Artificial Sequence
  Synthetic
KTKRKRKKQRVKIIFVQNM

SEQ ID NO: 34 - protein, 19 aa, Artificial Sequence
  Synthetic
KTKRKRKKQRVKIAFVQNM

SEQ ID NO: 35 - protein, 16 aa, Artificial Sequence
  Synthetic
KTKRKRKKQRVKIAYE

SEQ ID NO: 36 - protein, 15 aa, Artificial Sequence
  Synthetic
KTKRKRKKQRVKIAY

SEQ ID NO: 37 - protein, 14 aa, Artificial Sequence
  Synthetic
KTKRKRKKQRVKIA

SEQ ID NO: 38 - protein, 13 aa, Artificial Sequence
  Synthetic
KTKRKRKKQRVKI

SEQ ID NO: 39 - protein, 19 aa, Artificial Sequence
  Synthetic
KTKRKRKKQRVKIAYVQNM

SEQ ID NO: 40 - protein, 19 aa, Artificial Sequence
  Synthetic
KTKRKRKKQRVKIAYEQNM

SEQ ID NO: 41 - protein, 19 aa, Artificial Sequence
  Synthetic
KTKRKRKKQRVKIAYEENM

SEQ ID NO: 42 - protein, 19 aa, Artificial Sequence
  Synthetic
KTKRKRKKQRVKIAYEEIM

SEQ ID NO: 43 - protein, 13 aa, Artificial Sequence
  Synthetic
VKIAYEEIFVQNM

SEQ ID NO: 44 - protein, 14 aa, Artificial Sequence
  Synthetic
RVKIAYEEIFVQNM

SEQ ID NO: 45 - protein, 23 aa, Artificial Sequence
  Synthetic
KTKRKRKKQRVKIAYEEIFVQAA

SEQ ID NO: 46 - protein, 23 aa, Artificial Sequence
  Synthetic
KTKRKRKKQRVKIAYEEIFAANM

SEQ ID NO: 47 - protein, 23 aa, Artificial Sequence
  Synthetic
KTKRKRKKQRVKIAYEEAAVQNM

SEQ ID NO: 48 - protein, 23 aa, Artificial Sequence
  Synthetic
KTKRKRKKQRVKIAYAAIFVQNM

SEQ ID NO: 49 - protein, 23 aa, Artificial Sequence
  Synthetic
KTKRKRKKQRVKIAAEEIFVQNM

SEQ ID NO: 50 - protein, 23 aa, Artificial Sequence
  Synthetic
KTKRKRKKQRVAAAYEEIFVQNM

SEQ ID NO: 51 - protein, 24 aa, Artificial Sequence
  Synthetic
  MISC_FEATURE (1)..(1): Xaa = Acylated Lysine
  MISC_FEATURE (24)..(24): The side chain of Lysine at position 24 is covalently bound to an Ida linker forming a homodimer of residues 1 through 24.
XTKRKRKKQRVKIAYEEIFVQNMK

SEQ ID NO: 52 - protein, 19 aa, Artificial Sequence
  Synthetic
KTKRKRVKIAYEEIFVQNM

SEQ ID NO: 53 - protein, 23 aa, Artificial Sequence
  Synthetic
KTKRKRKAARVKIAYEEIFVQNM

SEQ ID NO: 54 - protein, 23 aa, Artificial Sequence
  Synthetic
KTKRKAAKQRVKIAYEEIFVQNM

SEQ ID NO: 55 - protein, 23 aa, Artificial Sequence
  Synthetic
KTKAARKKQRVKIAYEEIFVQNM

SEQ ID NO: 56 - protein, 23 aa, Artificial Sequence
  Synthetic
KAARKRKKQRVKIAYEEIFVQNM

SEQ ID NO: 57 - protein, 19 aa, Artificial Sequence
  Synthetic
KTKKQRVKIAYEEIFVQNM

SEQ ID NO: 58 - protein, 19 aa, Artificial Sequence
  Synthetic
KTKRKRKKIAYEEIFVQNM

SEQ ID NO: 59 - protein, 24 aa, Artificial Sequence
  Synthetic
  MISC_FEATURE (1)..(1): Xaa = 6-Aminohexanoic acid
  MISC_FEATURE (1)..(1): The amino  group of 6-aminohexanoic acid is covalently bound to an Ida linker forming a homodimer of residues 1 through 24.
XRTRRRRRRQRVRIAYEEIFVRNM

SEQ ID NO: 60 - protein, 19 aa, Artificial Sequence
  Synthetic
TKRKRKKQRVKIAYEEIFV

SEQ ID NO: 61 - protein, 18 aa, Artificial Sequence
  Synthetic
KRKRKKQRVKIAYEEIFV

SEQ ID NO: 62 - protein, 17 aa, Artificial Sequence
  Synthetic
RKRKKQRVKIAYEEIFV

SEQ ID NO: 63 - protein, 16 aa, Artificial Sequence
  Synthetic
KRKKQRVKIAYEEIFV

SEQ ID NO: 64 - protein, 15 aa, Artificial Sequence
  Synthetic
RKKQRVKIAYEEIFV

SEQ ID NO: 65 - protein, 18 aa, Artificial Sequence
  Synthetic
TKRKRKKQRVKIAYEEIF

SEQ ID NO: 66 - protein, 17 aa, Artificial Sequence
  Synthetic
KRKRKKQRVKIAYEEIF

SEQ ID NO: 67 - protein, 16 aa, Artificial Sequence
  Synthetic
RKRKKQRVKIAYEEIF

SEQ ID NO: 68 - protein, 15 aa, Artificial Sequence
  Synthetic
KRKKQRVKIAYEEIF

SEQ ID NO: 69 - protein, 14 aa, Artificial Sequence
  Synthetic
RKKQRVKIAYEEIF

SEQ ID NO: 70 - protein, 13 aa, Artificial Sequence
  Synthetic
KKQRVKIAYEEIF

SEQ ID NO: 71 - protein, 23 aa, Artificial Sequence
  Synthetic
KKKKKMPKLRFASRIRKIRKKQF

SEQ ID NO: 72 - protein, 23 aa, Artificial Sequence
  Synthetic
AAKRKRKKQRVKIAYEEIFVQNM

SEQ ID NO: 73 - protein, 23 aa, Artificial Sequence
  Synthetic
KTKRKRKKQAAKIAYEEIFVQNM

SEQ ID NO: 74 - protein, 20 aa, Artificial Sequence
  Synthetic
TKRKRKKQQRVKIAYEEIFV

SEQ ID NO: 75 - protein, 19 aa, Artificial Sequence
  Synthetic
KRKRKKQQRVKIAYEEIFV

SEQ ID NO: 76 - protein, 18 aa, Artificial Sequence
  Synthetic
RKRKKQQRVKIAYEEIFV

SEQ ID NO: 77 - protein, 17 aa, Artificial Sequence
  Synthetic
KRKKQQRVKIAYEEIFV

SEQ ID NO: 78 - protein, 16 aa, Artificial Sequence
  Synthetic
RKKQQRVKIAYEEIFV

SEQ ID NO: 79 - protein, 15 aa, Artificial Sequence
  Synthetic
KTKRKRKKQRVKIIF

SEQ ID NO: 80 - protein, 14 aa, Artificial Sequence
  Synthetic
TKRKRKKQRVKIIF

SEQ ID NO: 81 - protein, 13 aa, Artificial Sequence
  Synthetic
KRKRKKQRVKIIF

SEQ ID NO: 82 - protein, 12 aa, Artificial Sequence
  Synthetic
RKRKKQRVKIIF

SEQ ID NO: 83 - protein, 11 aa, Artificial Sequence
  Synthetic
KRKKQRVKIIF

SEQ ID NO: 84 - protein, 10 aa, Artificial Sequence
  Synthetic
RKKQRVKIIF

SEQ ID NO: 85 - protein, 16 aa, Artificial Sequence
  Synthetic
KTKRKRKKQRVKIIFV

SEQ ID NO: 86 - protein, 15 aa, Artificial Sequence
  Synthetic
TKRKRKKQRVKIIFV

SEQ ID NO: 87 - protein, 14 aa, Artificial Sequence
  Synthetic
KRKRKKQRVKIIFV

SEQ ID NO: 88 - protein, 12 aa, Artificial Sequence
  Synthetic
KRKKQRVKIIFV

SEQ ID NO: 89 - protein, 11 aa, Artificial Sequence
  Synthetic
RKKQRVKIIFV

SEQ ID NO: 90 - protein, 13 aa, Artificial Sequence
  Synthetic
RKRKKQRVKIIFV

SEQ ID NO: 91 - protein, 16 aa, Artificial Sequence
  Synthetic
  MISC_FEATURE (16)..(16): Dimeric peptide wherein the C terminus of Valine in position 16 of one peptide chain is covalently bound to one of the amino groups of a Lysine linker and Val of the remaining peptide chain is covalently bound to the other amino group of the Lysine
KTKRKRKKQRVKIIFV

SEQ ID NO: 92 - protein, 15 aa, Artificial Sequence
  Synthetic
  MISC_FEATURE (15)..(15): Dimeric peptide wherein the C terminus of Valine in position 15 of one peptide chain is covalently bound to one of the amino groups of a Lysine linker and Val of the remaining peptide chain is covalently bound to the other amino group of the Lysine
TKRKRKKQRVKIIFV

SEQ ID NO: 93 - protein, 14 aa, Artificial Sequence
  Synthetic
  MISC_FEATURE (14)..(14): Dimeric peptide wherein the C terminus of Valine in position 14 of one peptide chain is covalently bound to one of the amino groups of a Lysine linker and Val of the remaining peptide chain is covalently bound to the other amino group of the Lysine
KRKRKKQRVKIIFV

SEQ ID NO: 94 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RKRKKQRVKIIFV

SEQ ID NO: 95 - protein, 12 aa, Artificial Sequence
  Synthetic
  MISC_FEATURE (12)..(12): Dimeric peptide wherein the C terminus of Valine in position 12 of one peptide chain is covalently bound to one of the amino groups of a Lysine linker and Val of the remaining peptide chain is covalently bound to the other amino group of the Lysine
KRKKQRVKIIFV

SEQ ID NO: 96 - protein, 11 aa, Artificial Sequence
  Synthetic
  MISC_FEATURE (11)..(11): Dimeric peptide wherein the C terminus of Valine in position 11 of one peptide chain is covalently bound to one of the amino groups of a Lysine linker and Val of the remaining peptide chain is covalently bound to the other amino group of the Lysine
RKKQRVKIIFV

SEQ ID NO: 97 - protein, 15 aa, Artificial Sequence
  Synthetic
  MISC_FEATURE (15)..(15): Dimeric peptide wherein the C terminus of Phe in position 15 of one peptide chain is covalently bound to one of the amino groups of a Lysine linker and Phe of the remaining peptide chain is covalently bound to the other amino group of the Lysine linker.
KTKRKRKKQRVKIIF

SEQ ID NO: 98 - protein, 14 aa, Artificial Sequence
  Synthetic
  MISC_FEATURE (14)..(14): Dimeric peptide wherein the C terminus of Phe in position 14 of one peptide chain is covalently bound to one of the amino groups of a Lysine linker and Phe of the remaining peptide chain is covalently bound to the other amino group of the Lysine linker.
TKRKRKKQRVKIIF

SEQ ID NO: 99 - protein, 13 aa, Artificial Sequence
  Synthetic
  MISC_FEATURE (13)..(13): Dimeric peptide wherein the C terminus of Phe in position 13 of one peptide chain is covalently bound to one of the amino groups of a Lysine linker and Phe of the remaining peptide chain is covalently bound to the other amino group of the Lysine linker.
KRKRKKQRVKIIF

SEQ ID NO: 100 - protein, 12 aa, Artificial Sequence
  Synthetic
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
RKRKKQRVKIIF

SEQ ID NO: 101 - protein, 11 aa, Artificial Sequence
  Synthetic
  MISC_FEATURE (11)..(11): Dimeric peptide wherein the C terminus of Phe in position 11 of one peptide chain is covalently bound to one of the amino groups of a Lysine linker and Phe of the remaining peptide chain is covalently bound to the other amino group of the Lysine linker.
KRKKQRVKIIF

SEQ ID NO: 102 - protein, 10 aa, Artificial Sequence
  Synthetic
  MISC_FEATURE (10)..(10): Dimeric peptide wherein the C terminus of Phe in position 10 of one peptide chain is covalently bound to one of the amino groups of a Lysine linker and Phe of the remaining peptide chain is covalently bound to the other amino group of the Lysine linker.
RKKQRVKIIF

SEQ ID NO: 103 - protein, 19 aa, Artificial Sequence
  Synthetic
  MISC_FEATURE (19)..(19): Dimeric peptide wherein the C terminus of Met in position 19 of one peptide chain is covalently bound to one of the amino groups of a Lysine linker and Met of the remaining peptide chain is covalently bound to the other amino group of the Lysine linker.
KTKRKRKKQRVKIIFVQNM

SEQ ID NO: 104 - protein, 19 aa, Artificial Sequence
  Synthetic
  MISC_FEATURE (1)..(1): Xaa = Acylated Lysine
  MISC_FEATURE (19)..(19): Dimeric peptide wherein the C terminus of Met in position 19 of one peptide chain is covalently bound to one of the amino groups of a Lysine linker and Met of the remaining peptide chain is covalently bound to the other amino group of the Lysine linker.
XTKRKRKKQRVKIIFVQNM

SEQ ID NO: 105 - protein, 19 aa, Artificial Sequence
  Synthetic
KTKRKRKKQRVKIIFVQNA

SEQ ID NO: 106 - protein, 19 aa, Artificial Sequence
  Synthetic
KTKRKRKKQRVKIIFVQAM

SEQ ID NO: 107 - protein, 19 aa, Artificial Sequence
  Synthetic
KTKRKRKKQRVKIIFVANM

SEQ ID NO: 108 - protein, 19 aa, Artificial Sequence
  Synthetic
KTKRKRKKQRVKIIFAQNM

SEQ ID NO: 109 - protein, 19 aa, Artificial Sequence
  Synthetic
KTKRKRKKQRVKIIAVQNM

SEQ ID NO: 110 - protein, 19 aa, Artificial Sequence
  Synthetic
KTKRKRKKQRVKIAFVQNM

SEQ ID NO: 111 - protein, 19 aa, Artificial Sequence
  Synthetic
KTKRKRKKQRVKAIFVQNM

SEQ ID NO: 112 - protein, 19 aa, Artificial Sequence
  Synthetic
KTKRKRKKQRVAIIFVQNM

SEQ ID NO: 113 - protein, 19 aa, Artificial Sequence
  Synthetic
KTKRKRKKQRAKIIFVQNM

SEQ ID NO: 114 - protein, 19 aa, Artificial Sequence
  Synthetic
KTKRKRKKQAVKIIFVQNM

SEQ ID NO: 115 - protein, 19 aa, Artificial Sequence
  Synthetic
KTKRKRKKARVKIIFVQNM

SEQ ID NO: 116 - protein, 19 aa, Artificial Sequence
  Synthetic
KTKRKRKAQRVKIIFVQNM

SEQ ID NO: 117 - protein, 19 aa, Artificial Sequence
  Synthetic
KTKRKRAKQRVKIIFVQNM

SEQ ID NO: 118 - protein, 19 aa, Artificial Sequence
  Synthetic
KTKRKAKKQRVKIIFVQNM

SEQ ID NO: 119 - protein, 19 aa, Artificial Sequence
  Synthetic
KTKRARKKQRVKIIFVQNM

SEQ ID NO: 120 - protein, 19 aa, Artificial Sequence
  Synthetic
KTKAKRKKQRVKIIFVQNM

SEQ ID NO: 121 - protein, 19 aa, Artificial Sequence
  Synthetic
KTARKRKKQRVKIIFVQNM

SEQ ID NO: 122 - protein, 19 aa, Artificial Sequence
  Synthetic
KAKRKRKKQRVKIIFVQNM

SEQ ID NO: 123 - protein, 19 aa, Artificial Sequence
  Synthetic
ATKRKRKKQRVKIIFVQNM

SEQ ID NO: 124 - protein, 20 aa, Artificial Sequence
  Synthetic
  MISC_FEATURE (20)..(20): Dimeric peptide wherein the C terminus of Valine in position 20 of one peptide chain is covalently bound to one of the amino groups of a Lysine linker and Val of the remaining peptide chain is covalently bound to the other amino group of the Lysine
KTKRKRKKQRVKIAYEEIFV

SEQ ID NO: 125 - protein, 17 aa, Artificial Sequence
  Synthetic
  MISC_FEATURE (17)..(17): Dimeric peptide wherein the C terminus of Phe in position 17 of one peptide chain is covalently bound to one of the amino groups of a Lysine linker and Phe of the remaining peptide chain is covalently bound to the other amino group of the Lysine linker.
KRKRKKQRVKIAYEEIF

SEQ ID NO: 126 - protein, 18 aa, Artificial Sequence
  Synthetic
  MISC_FEATURE (18)..(18): Dimeric peptide wherein the C terminus of Phe in position 18 of one peptide chain is covalently bound to one of the amino groups of a Lysine linker and Phe of the remaining peptide chain is covalently bound to the other amino group of the Lysine linker.
TKRKRKKQRVKIAYEEIF

SEQ ID NO: 127 - protein, 19 aa, Artificial Sequence
  Synthetic
  MISC_FEATURE (19)..(19): Dimeric peptide wherein the C terminus of Phe in position 19 of one peptide chain is covalently bound to one of the amino groups of a Lysine linker and Phe of the remaining peptide chain is covalently bound to the other amino group of the Lysine linker.
KTKRKRKKQRVKIAYEEIF

SEQ ID NO: 128 - protein, 17 aa, Artificial Sequence
  Synthetic
  MISC_FEATURE (14)..(14): Xaa = 6-Aminohexanoic acid
  MISC_FEATURE (15)..(15): Xaa = 6-Aminohexanoic acid
  MISC_FEATURE (17)..(17): Dimeric peptide wherein the C terminus of Phe in position 17 of one peptide chain is covalently bound to one of the amino groups of a Lysine linker and Phe of the remaining peptide chain is covalently bound to the other amino group of the Lysine linker.
KTKRKRKKQRVKIXXIF

SEQ ID NO: 129 - protein, 16 aa, Artificial Sequence
  Synthetic
  MISC_FEATURE (13)..(13): Xaa = 6-Aminohexanoic acid
  MISC_FEATURE (14)..(14): Xaa = 6-Aminohexanoic acid
  MISC_FEATURE (16)..(16): Dimeric peptide wherein the C terminus of Phe in position 16 of one peptide chain is covalently bound to one of the amino groups of a Lysine linker and Phe of the remaining peptide chain is covalently bound to the other amino group of the Lysine linker.
TKRKRKKQRVKIXXIF

SEQ ID NO: 130 - protein, 14 aa, Artificial Sequence
  Synthetic
  MISC_FEATURE (14)..(14): Dimeric peptide wherein the C terminus of Phe in position 14 of one peptide chain is covalently bound to one of the amino groups of a Lysine linker and Phe of the remaining peptide chain is covalently bound to the other amino group of the Lysine linker.
TRRRRRRQRVRIIF

SEQ ID NO: 131 - protein, 13 aa, Artificial Sequence
  Synthetic
  MISC_FEATURE (13)..(13): Dimeric peptide wherein the C terminus of Phe in position 13 of one peptide chain is covalently bound to one of the amino groups of a Lysine linker and Phe of the remaining peptide chain is covalently bound to the other amino group of the Lysine linker.
RRRRRRQRVRIIF

SEQ ID NO: 132 - protein, 12 aa, Artificial Sequence
  Synthetic
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
RRRRRQRVRIIF

SEQ ID NO: 133 - protein, 11 aa, Artificial Sequence
  Synthetic
  MISC_FEATURE (11)..(11): Dimeric peptide wherein the C terminus of Phe in position 11 of one peptide chain is covalently bound to one of the amino groups of a Lysine linker and Phe of the remaining peptide chain is covalently bound to the other amino group of the Lysine linker.
RRRRQRVRIIF

SEQ ID NO: 134 - protein, 10 aa, Artificial Sequence
  Synthetic
  MISC_FEATURE (10)..(10): Dimeric peptide wherein the C terminus of Phe in position 10 of one peptide chain is covalently bound to one of the amino groups of a Lysine linker and Phe of the remaining peptide chain is covalently bound to the other amino group of the Lysine linker.
RRRQRVRIIF

SEQ ID NO: 135 - protein, 15 aa, Artificial Sequence
  Synthetic
  MISC_FEATURE (12)..(12): Xaa = 6-Aminohexanoic acid
  MISC_FEATURE (13)..(13): Xaa = 6-Aminohexanoic acid
  MISC_FEATURE (15)..(15): Dimeric peptide wherein the C terminus of Phe in position 15 of one peptide chain is covalently bound to one of the amino groups of a Lysine linker and Phe of the remaining peptide chain is covalently bound to the other amino group of the Lysine linker.
KRKRKKQRVKIXXIF

SEQ ID NO: 136 - protein, 19 aa, Artificial Sequence
  Synthetic
KTKRKRKKQRVKRAYEEIF

SEQ ID NO: 137 - protein, 19 aa, Artificial Sequence
  Synthetic
KTKRKRIKQRVKRAYEEIF

SEQ ID NO: 138 - protein, 13 aa, Artificial Sequence
  Synthetic
  MISC_FEATURE (13)..(13): Dimeric peptide wherein the C terminus of Ala in position 13 of one peptide chain is covalently bound to one of the amino groups of a Lysine linker and Ala of the remaining peptide chain is covalently bound to the other amino group of the Lysine linker.
RKRKKQRVKIIFA

SEQ ID NO: 139 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RKRKKQRVKIIAV

SEQ ID NO: 140 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RKRKKQRVKIAFV

SEQ ID NO: 141 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RKRKKQRVKAIFV

SEQ ID NO: 142 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RKRKKQRVAIIFV

SEQ ID NO: 143 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RKRKKQRAKIIFV

SEQ ID NO: 144 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RKRKKQAVKIIFV

SEQ ID NO: 145 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RKRKKARVKIIFV

SEQ ID NO: 146 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RKRKAQRVKIIFV

SEQ ID NO: 147 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RKRAKQRVKIIFV

SEQ ID NO: 148 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RKAKKQRVKIIFV

SEQ ID NO: 149 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
RARKKQRVKIIFV

SEQ ID NO: 150 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
AKRKKQRVKIIFV

SEQ ID NO: 151 - protein, 27 aa, Artificial Sequence
  Synthetic
NKRKKQRVKIIFVKVFIIKVRQKKRKN

SEQ ID NO: 152 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
QKRKKQRVKIIFV

SEQ ID NO: 153 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
GKRKKQRVKIIFV

SEQ ID NO: 154 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
HKRKKQRVKIIFV

SEQ ID NO: 155 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
IKRKKQRVKIIFV

SEQ ID NO: 156 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
LKRKKQRVKIIFV

SEQ ID NO: 157 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
FKRKKQRVKIIFV

SEQ ID NO: 158 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
SKRKKQRVKIIFV

SEQ ID NO: 159 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
TKRKKQRVKIIFV

SEQ ID NO: 160 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
YKRKKQRVKIIFV

SEQ ID NO: 161 - protein, 13 aa, Artificial Sequence
  Synthetic
  MISC_FEATURE (13)..(13): Dimeric peptide wherein the C terminus of Valine in position 13 of one peptide chain is covalently bound to one of the amino groups of a Lysine linker and Val of the remaining peptide chain is covalently bound to the other amino group of the Lysine
VKRKKQRVKIIFV

SEQ ID NO: 162 - protein, 10 aa, Artificial Sequence
  Synthetic
  MISC_FEATURE (10)..(10): Dimeric peptide wherein the C terminus of Valine in position 10 of one peptide chain is covalently bound to one of the amino groups of a Lysine linker and Val of the remaining peptide chain is covalently bound to the other amino group of the Lysine
KKQRVKIIFV

SEQ ID NO: 163 - protein, 9 aa, Artificial Sequence
  Synthetic
  MISC_FEATURE (9)..(9): Dimeric peptide wherein the C terminus of Valine in position 9 of one peptide chain is covalently bound to one of the amino groups of a Lysine linker and Val of the remaining peptide chain is covalently bound to the other amino group of the Lysine linker.
KQRVKIIFV

SEQ ID NO: 164 - protein, 9 aa, Artificial Sequence
  Synthetic
  MISC_FEATURE (9)..(9): Dimeric peptide wherein the C terminus of Phe in position 9 of one peptide chain is covalently bound to one of the amino groups of a Lysine linker and Phe of the remaining peptide chain is covalently bound to the other amino group of the Lysine linker.
KKQRVKIIF

SEQ ID NO: 165 - protein, 8 aa, Artificial Sequence
  Synthetic
  MISC_FEATURE (8)..(8): Dimeric peptide wherein the C terminus of Phe in position 8 of one peptide chain is covalently bound to one of the amino groups of a Lysine linker and Phe of the remaining peptide chain is covalently bound to the other amino group of the Lysine linker.
KQRVKIIF

SEQ ID NO: 166 - protein, 15 aa, Artificial Sequence
  Synthetic
  MISC_FEATURE (1)..(1): Xaa=Biotinylated Threonine
XKRKRKKQRVKIIFV

SEQ ID NO: 167 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRRQRVRIIFK

SEQ ID NO: 168 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an GP3 linker forming a homodimer of residues 1 through 13.
XRRRRQRVRIIFK

SEQ ID NO: 169 - protein, 13 aa, Artificial Sequence
  Synthetic
  MISC_FEATURE (1)..(1): Xaa = Acylated Lysine
  MISC_FEATURE (1)..(1): The side chain of the acylated Lysine at position 1 is covalently bound to an Ida linker forming a homodimer of residues 1 through 13.
XRRRRRQRVRIIF

SEQ ID NO: 170 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida PEG (20kDA) linker forming a homodimer of residues 1 through 13.
XRRRRQRVRIIFK

SEQ ID NO: 171 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an GP3 PEG (20 kDa) linker forming a homodimer of residues 1 through 13.
XRRRRQRVRIIFK

SEQ ID NO: 172 - protein, 13 aa, Artificial Sequence
  Synthetic
  MISC_FEATURE (1)..(1): Xaa = Acylated Lysine
  MISC_FEATURE (1)..(1): The side chain of the acylated Lysine at position 1 is covalently bound to an Ida PEG (20 kDa) linker forming a homodimer of residues 1 through 13.
XRRRRRQRVRIIF

SEQ ID NO: 173 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQRVRIIF

SEQ ID NO: 174 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XARRRQRVRIIF

SEQ ID NO: 175 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRARRQRVRIIF

SEQ ID NO: 176 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRARQRVRIIF

SEQ ID NO: 177 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRAQRVRIIF

SEQ ID NO: 178 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQAVRIIF

SEQ ID NO: 179 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQRVAIIF

SEQ ID NO: 180 - protein, 13 aa, Artificial Sequence
  Synthetic
  MISC_FEATURE (13)..(13): Dimeric peptide wherein the C terminus of Arg in position 13 of one peptide chain is covalently bound to one of the amino groups of a Lysine linker and Arg of the remaining peptide chain is covalently bound to the other amino group of the Lysine linker.
RQRIRVRIRFRVR

SEQ ID NO: 181 - protein, 13 aa, Artificial Sequence
  Synthetic
  MISC_FEATURE (13)..(13): Dimeric peptide wherein the C terminus of Arg in position 13 of one peptide chain is covalently bound to one of the amino groups of a Lysine linker and Arg of the remaining peptide chain is covalently bound to the other amino group of the Lysine linker.
FRRVRRIVRRIQR

SEQ ID NO: 182 - protein, 13 aa, Artificial Sequence
  Synthetic
  MISC_FEATURE (13)..(13): Dimeric peptide wherein the C terminus of Val in position 13 of one peptide chain is covalently bound to one of the amino groups of a Lysine linker and Val of the remaining peptide chain is covalently bound to the other amino group of the Lysine linker.
RIQRRFRRVIRRV

SEQ ID NO: 183 - protein, 12 aa, Artificial Sequence
  Synthetic
  MISC_FEATURE (12)..(12): Dimeric peptide wherein the C terminus of Arg in position 12 of one peptide chain is covalently bound to one of the amino groups of a Lysine linker and Arg of the remaining peptide chain is covalently bound to the other amino group of the Lysine linker.
RFVRIRIRQRRR

SEQ ID NO: 184 - protein, 12 aa, Artificial Sequence
  Synthetic
  MISC_FEATURE (1)..(1): Xaa = Acylated Histidine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQRVRIIF

SEQ ID NO: 185 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XHRRRQRVRIIF

SEQ ID NO: 186 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRHRRQRVRIIF

SEQ ID NO: 187 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRHRQRVRIIF

SEQ ID NO: 188 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRHQRVRIIF

SEQ ID NO: 189 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQHVRIIF

SEQ ID NO: 190 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQRVHIIF

SEQ ID NO: 191 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XARRRQRVRIIF

SEQ ID NO: 192 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRARRQRVRIIF

SEQ ID NO: 193 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRARQRVRIIF

SEQ ID NO: 194 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRAQRVRIIF

SEQ ID NO: 195 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQAVRIIF

SEQ ID NO: 196 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQRVAIIF

SEQ ID NO: 197 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XAARRQRVRIIF

SEQ ID NO: 198 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XARARQRVRIIF

SEQ ID NO: 199 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XARRAQRVRIIF

SEQ ID NO: 200 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XARRRQAVRIIF

SEQ ID NO: 201 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XARRRQRVAIIF

SEQ ID NO: 202 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRAARQRVRIIF

SEQ ID NO: 203 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRARAQRVRIIF

SEQ ID NO: 204 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRARRQAVRIIF

SEQ ID NO: 205 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRARRQRVAIIF

SEQ ID NO: 206 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRAAQRVRIIF

SEQ ID NO: 207 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRARQAVRIIF

SEQ ID NO: 208 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRARQRVAIIF

SEQ ID NO: 209 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRAQAVRIIF

SEQ ID NO: 210 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRAQRVAIIF

SEQ ID NO: 211 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQAVAIIF

SEQ ID NO: 212 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XARRRQRVAIIF

SEQ ID NO: 213 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRARRQRVAIIF

SEQ ID NO: 214 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRARQRVAIIF

SEQ ID NO: 215 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRAQRVAIIF

SEQ ID NO: 216 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQAVAIIF

SEQ ID NO: 217 - protein, 12 aa, Artificial Sequence
  Synthetic
  MISC_FEATURE (1)..(1): Xaa = Acylated Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XHHHHQHVAIIF

SEQ ID NO: 218 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XARRRQAVAIIF

SEQ ID NO: 219 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRARRQAVAIIF

SEQ ID NO: 220 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRARQAVAIIF

SEQ ID NO: 221 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRAQAVAIIF

SEQ ID NO: 222 - protein, 12 aa, Artificial Sequence
  Synthetic
  MISC_FEATURE (1)..(1): Xaa = Acylated Histidine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XHHHHQAVAIIF

SEQ ID NO: 223 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIGFK

SEQ ID NO: 224 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIHFK

SEQ ID NO: 225 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAINFK

SEQ ID NO: 226 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAITFK

SEQ ID NO: 227 - protein, 13 aa, Artificial Sequence
  Synthetic
  MISC_FEATURE (1)..(1): Xaa = Acylated Alanine
  MISC_FEATURE (13)..(13): The side chain of Lysine at position 13  is covalently bound to an Ida linker forming a homodimer of residues 1 through 13.
XRRRRQRVAIWFK

SEQ ID NO: 228 - protein, 13 aa, Artificial Sequence
  Synthetic
  MISC_FEATURE (1)..(1): Xaa = Acylated Alanine
  MISC_FEATURE (13)..(13): The side chain of Lysine at position 13  is covalently bound to an Ida linker forming a homodimer of residues 1 through 13.
XRRRRQRVAIYFK

SEQ ID NO: 229 - protein, 12 aa, Artificial Sequence
  Synthetic
  MISC_FEATURE (1)..(1): Xaa = Acylated Histidine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XHRRRQRVRIIF

SEQ ID NO: 230 - protein, 12 aa, Artificial Sequence
  Synthetic
  MISC_FEATURE (1)..(1): Xaa = Acylated Histidine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRHRRQRVRIIF

SEQ ID NO: 231 - protein, 12 aa, Artificial Sequence
  Synthetic
  MISC_FEATURE (1)..(1): Xaa = Acylated Histidine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRHRQRVRIIF

SEQ ID NO: 232 - protein, 12 aa, Artificial Sequence
  Synthetic
  MISC_FEATURE (1)..(1): Xaa = Acylated Histidine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRHQRVRIIF

SEQ ID NO: 233 - protein, 12 aa, Artificial Sequence
  Synthetic
  MISC_FEATURE (1)..(1): Xaa = Acylated Histidine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQHVRIIF

SEQ ID NO: 234 - protein, 12 aa, Artificial Sequence
  Synthetic
  MISC_FEATURE (1)..(1): Xaa = Acylated Histidine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQRVHIIF

SEQ ID NO: 235 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XHHRRQRVRIIF

SEQ ID NO: 236 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XHRHRQRVRIIF

SEQ ID NO: 237 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XHRRHQRVRIIF

SEQ ID NO: 238 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XHRRRQHVRIIF

SEQ ID NO: 239 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XHRRRQRVHIIF

SEQ ID NO: 240 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRHHRQRVRIIF

SEQ ID NO: 241 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRHRHQRVRIIF

SEQ ID NO: 242 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRHRRQHVRIIF

SEQ ID NO: 243 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRHRRQRVHIIF

SEQ ID NO: 244 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRHRQHVRIIF

SEQ ID NO: 245 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRHRQRVHIIF

SEQ ID NO: 246 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRHQHVRIIF

SEQ ID NO: 247 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRHQRVHIIF

SEQ ID NO: 248 - protein, 12 aa, Artificial Sequence
  Synthetic
  MISC_FEATURE (1)..(1): Xaa = Acylated Arginine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQHVHIIF

SEQ ID NO: 249 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRRQAVAIEFK

SEQ ID NO: 250 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRRQAVAIHFK

SEQ ID NO: 251 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRRQAVAIPFK

SEQ ID NO: 252 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIIFK

SEQ ID NO: 253 - protein, 13 aa, Artificial Sequence
  Synthetic
  MISC_FEATURE (1)..(1): Xaa = Acylated Alanine
  MISC_FEATURE (12)..(12): Xaa = the “D” enantiomer of Phenylalanine
  MISC_FEATURE (13)..(13): The side chain of Lysine at position 13 is covalently bound to an Ida linker forming a homodimer of residues 1 through 13.
XRRRRQRVAIIFK

SEQ ID NO: 254 - protein, 13 aa, Artificial Sequence
  Synthetic
  MISC_FEATURE (1)..(1): Xaa = Acylated Alanine
  MISC_FEATURE (11)..(11): Xaa = the “D” enantiomer of Isoleucine
  MISC_FEATURE (13)..(13): The side chain of Lysine at position 13 is covalently bound to an Ida linker forming a homodimer of residues 1 through 13.
XRRRRQRVAIXFK

SEQ ID NO: 255 - protein, 13 aa, Artificial Sequence
  Synthetic
  MISC_FEATURE (1)..(1): Xaa = Acylated Alanine
  MISC_FEATURE (10)..(10): Xaa = the “D” enantiomer of Ile
  MISC_FEATURE (13)..(13): The side chain of Lysine at position 13 is covalently bound to an Ida linker forming a homodimer of residues 1 through 13.
XRRRRQRVAXIFK

SEQ ID NO: 256 - protein, 13 aa, Artificial Sequence
  Synthetic
  MISC_FEATURE (1)..(1): Xaa = Acylated Alanine
  MISC_FEATURE (9)..(9): Xaa = the “D” enantiomer of Ala
  MISC_FEATURE (13)..(13): The side chain of Lysine at position 13 is covalently bound to an Ida linker forming a homodimer of residues 1 through 13.
XRRRRQRVXIIFK

SEQ ID NO: 257 - protein, 13 aa, Artificial Sequence
  Synthetic
  MISC_FEATURE (1)..(1): Xaa = Acylated Alanine
  MISC_FEATURE (8)..(8): Xaa = the “D” enantiomer of Val
  MISC_FEATURE (13)..(13): The side chain of Lysine at position 13 is covalently bound to an Ida linker forming a homodimer of residues 1 through 13.
XRRRRQRXAIIFK

SEQ ID NO: 258 - protein, 13 aa, Artificial Sequence
  Synthetic
  MISC_FEATURE (1)..(1): Xaa = Acylated Alanine
  MISC_FEATURE (7)..(7): Xaa = the “D” enantiomer of Arg
  MISC_FEATURE (13)..(13): The side chain of Lysine at position 13 is covalently bound to an Ida linker forming a homodimer of residues 1 through 13.
XRRRRQXVAIIFK

SEQ ID NO: 259 - protein, 13 aa, Artificial Sequence
  Synthetic
  MISC_FEATURE (1)..(1): Xaa = Acylated Alanine
  MISC_FEATURE (6)..(6): Xaa = the “D” enantiomer of Gln
  MISC_FEATURE (13)..(13): The side chain of Lysine at position 13 is covalently bound to an Ida linker forming a homodimer of residues 1 through 13.
XRRRRXRVAIIFK

SEQ ID NO: 260 - protein, 13 aa, Artificial Sequence
  Synthetic
  MISC_FEATURE (1)..(1): Xaa = Acylated Alanine
  MISC_FEATURE (5)..(5): Xaa = the “D” enantiomer of Arg
  MISC_FEATURE (13)..(13): The side chain of Lysine at position 13 is covalently bound to an Ida linker forming a homodimer of residues 1 through 13.
XRRRXQRVAIIFK

SEQ ID NO: 261 - protein, 13 aa, Artificial Sequence
  Synthetic
  MISC_FEATURE (1)..(1): Xaa = Acylated Alanine
  MISC_FEATURE (4)..(4): Xaa = the “D” enantiomer of Arg
  MISC_FEATURE (13)..(13): The side chain of Lysine at position 13 is covalently bound to an Ida linker forming a homodimer of residues 1 through 13.
XRRXRQRVAIIFK

SEQ ID NO: 262 - protein, 13 aa, Artificial Sequence
  Synthetic
  MISC_FEATURE (1)..(1): Xaa = Acylated Alanine
  MISC_FEATURE (3)..(3): Xaa = the “D” enantiomer of Arg
  MISC_FEATURE (13)..(13): The side chain of Lysine at position 13 is covalently bound to an Ida linker forming a homodimer of residues 1 through 13.
XRXRRQRVAIIFK

SEQ ID NO: 263 - protein, 13 aa, Artificial Sequence
  Synthetic
  MISC_FEATURE (1)..(1): Xaa = Acylated Alanine
  MISC_FEATURE (2)..(2): Xaa = the “D” of Arg
  MISC_FEATURE (13)..(13): The side chain of Lysine at position 13 is covalently bound to an Ida linker forming a homodimer of residues 1 through 13.
XXRRRQRVAIIFK

SEQ ID NO: 264 - protein, 13 aa, Artificial Sequence
  Synthetic
  MISC_FEATURE (1)..(1): Xaa = Acylated  “D” enantiomer of Ala
  MISC_FEATURE (13)..(13): The side chain of Lysine at position 13 is covalently bound to an Ida linker forming a homodimer of residues 1 through 13.
XRRRRQRVAIIFK

SEQ ID NO: 265 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRRQAVAIGFK

SEQ ID NO: 266 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRRQAVAIWFK

SEQ ID NO: 267 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRRQAVAIYFK

SEQ ID NO: 268 - protein, 13 aa, Artificial Sequence
  Synthetic
  MISC_FEATURE (1)..(1): Xaa = Acylated  Alanine
  MISC_FEATURE (13)..(13): The side chain of Lysine at position 13 is covalently bound to an Ida linker forming a homodimer of residues 1 through 13.
XRRRRQRVAIPFK

SEQ ID NO: 269 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIEFK

SEQ ID NO: 270 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRRQAVAINFK

SEQ ID NO: 271 - protein, 12 aa, Artificial Sequence
  Synthetic
  MISC_FEATURE (1)..(1): Xaa = Acylated  Alanine
  MISC_FEATURE (12)..(12): Dimeric peptide wherein the C terminus of Phe in position 12 of one peptide chain is covalently bound to one of the amino groups of a Lysine linker and Phe of the remaining peptide chain is covalently bound to the other amino group of the Lysine linker.
XRRRRQRVAIYF

SEQ ID NO: 272 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XARRRQAVAINFK

SEQ ID NO: 273 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XARRRQAVAITFK

SEQ ID NO: 274 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XARRRQAVAIWFK

SEQ ID NO: 275 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XARRRQAVAIYFK

SEQ ID NO: 276 - protein, 13 aa, Artificial Sequence
  Synthetic
  MISC_FEATURE (1)..(1): Xaa = Acylated  Histidine
  MISC_FEATURE (13)..(13): The side chain of Lysine at position 13 is covalently bound to an Ida linker forming a homodimer of residues 1 through 13.
XRRRRQHVRIYFK

SEQ ID NO: 277 - protein, 13 aa, Artificial Sequence
  Synthetic
  MISC_FEATURE (1)..(1): Xaa = Acylated  Histidine
  MISC_FEATURE (13)..(13): The side chain of Lysine at position 13 is covalently bound to an Ida linker forming a homodimer of residues 1 through 13.
XRRRRQRVHIYFK

SEQ ID NO: 278 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRRQHVHIYFK

SEQ ID NO: 279 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRRQRVRIYFK

SEQ ID NO: 280 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRAQAVAIYFK

SEQ ID NO: 281 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRHQAVAIYFK

SEQ ID NO: 282 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRAQHVAIYFK

SEQ ID NO: 283 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRAQAVHIYFK

SEQ ID NO: 284 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRHQHVAIYFK

SEQ ID NO: 285 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRHQAVHIYFK

SEQ ID NO: 286 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRAQHVHIYFK

SEQ ID NO: 287 - protein, 13 aa, Artificial Sequence
  Synthetic
  MISC_FEATURE (1)..(1): Xaa = Acylated  Arginine
  MISC_FEATURE (13)..(13): The side chain of Lysine at position 13 is covalently bound to an Ida linker forming a homodimer of residues 1 through 13.
XRRRHQHVHIYFK

SEQ ID NO: 288 - protein, 13 aa, Artificial Sequence
  Synthetic
  MISC_FEATURE (1)..(1): Xaa = Acylated  Alanine
  MISC_FEATURE (11)..(11): Xaa = the “D” enantiomer of Tyr
  MISC_FEATURE (13)..(13): The side chain of Lysine at position 13 is covalently bound to an Ida linker forming a homodimer of residues 1 through 13.
XRRRRQRVAIXFK

SEQ ID NO: 289 - protein, 13 aa, Artificial Sequence
  Synthetic
  MISC_FEATURE (1)..(1): Xaa = Acylated  Alanine
  MISC_FEATURE (11)..(11): Xaa = 1-napthylalanine
  MISC_FEATURE (13)..(13): The side chain of Lysine at position 13 is covalently bound to an Ida linker forming a homodimer of residues 1 through 13.
XRRRRQRVAIXFK

SEQ ID NO: 290 - protein, 13 aa, Artificial Sequence
  Synthetic
  MISC_FEATURE (1)..(1): Xaa = Acylated Alanine
  MISC_FEATURE (11)..(11): Xaa = Phenylglycine
  MISC_FEATURE (13)..(13): The side chain of Lysine at position 13 is covalently bound to an Ida linker forming a homodimer of residues 1 through 13.
XRRRRQRVAIXFK

SEQ ID NO: 291 - protein, 13 aa, Artificial Sequence
  Synthetic
  MISC_FEATURE (1)..(1): Xaa = Acylated  Alanine
  MISC_FEATURE (11)..(11): Xaa = Beta-cyclohexylalanine
  MISC_FEATURE (13)..(13): The side chain of Lysine at position 13 is covalently bound to an Ida linker forming a homodimer of residues 1 through 13.
XRRRRQRVAIXFK

SEQ ID NO: 292 - protein, 13 aa, Artificial Sequence
  Synthetic
  MISC_FEATURE (1)..(1): Xaa = Acylated Alanine
  MISC_FEATURE (11)..(11): Xaa = Homophenylalanine
  MISC_FEATURE (13)..(13): The side chain of Lysine at position 13 is covalently bound to an Ida linker forming a homodimer of residues 1 through 13.
XRRRRQRVAIXFK

SEQ ID NO: 293 - protein, 13 aa, Artificial Sequence
  Synthetic
  MISC_FEATURE (1)..(1): Xaa = Acylated Alanine
  MISC_FEATURE (11)..(11): Xaa = the “D” enantiomer of Homophenylalanine
  MISC_FEATURE (13)..(13): The side chain of Lysine at position 13 is covalently bound to an Ida linker forming a homodimer of residues 1 through 13.
XRRRRQRVAIXFK

SEQ ID NO: 294 - protein, 13 aa, Artificial Sequence
  Synthetic
  MISC_FEATURE (1)..(1): Xaa = Acylated Alanine
  MISC_FEATURE (11)..(11): Xaa = 3,4-Dichlorophenylalanine
  MISC_FEATURE (13)..(13): The side chain of Lysine at position 13 is covalently bound to an Ida linker forming a homodimer of residues 1 through 13.
XRRRRQRVAIXFK

SEQ ID NO: 295 - protein, 13 aa, Artificial Sequence
  Synthetic
  MISC_FEATURE (1)..(1): Xaa = Acylated Alanine
  MISC_FEATURE (11)..(11): Xaa = 3,3-Diphenylphenylalanine
  MISC_FEATURE (13)..(13): The side chain of Lysine at position 13 is covalently bound to an Ida linker forming a homodimer of residues 1 through 13.
XRRRRQRVAIXFK

SEQ ID NO: 296 - protein, 13 aa, Artificial Sequence
  Synthetic
  MISC_FEATURE (1)..(1): Xaa = Acylated Alanine
  MISC_FEATURE (11)..(11): Xaa = 4-Chlorophenylalanine
  MISC_FEATURE (13)..(13): The side chain of Lysine at position 13 is covalently bound to an Ida linker forming a homodimer of residues 1 through 13.
XRRRRQRVAIXFK

SEQ ID NO: 297 - protein, 13 aa, Artificial Sequence
  Synthetic
  MISC_FEATURE (1)..(1): Xaa = Acylated Alanine
  MISC_FEATURE (11)..(11): Xaa = Nitrophenylalanine
  MISC_FEATURE (11)..(11): Xaa = 4-Nitrophenylalanine
  MISC_FEATURE (13)..(13): The side chain of Lysine at position 13 is covalently bound to an Ida linker forming a homodimer of residues 1 through 13.
XRRRRQRVAIXFK

SEQ ID NO: 298 - protein, 12 aa, Artificial Sequence
  Synthetic
  MISC_FEATURE (1)..(1): Xaa = Acylated Alanine
  MISC_FEATURE (10)..(10): Xaa = Alpha-aminoisobutyric acid
  MISC_FEATURE (12)..(12): The side chain of Lysine at position 12 is covalently bound to an Ida linker forming a homodimer of residues 1 through 12.
XRRRRQRVAXFK

SEQ ID NO: 299 - protein, 15 aa, Artificial Sequence
  Synthetic
  MISC_FEATURE (1)..(1): Xaa = Acylated Arginine
  MISC_FEATURE (15)..(15): The side chain of Lysine at position 15 is covalently bound to an Ida linker forming a homodimer of residues 1 through 15.
XRRRRQAVRIGIGFK

SEQ ID NO: 300 - protein, 15 aa, Artificial Sequence
  Synthetic
  MISC_FEATURE (1)..(1): Xaa = Acylated Arginine
  MISC_FEATURE (15)..(15): The side chain of Lysine at position 15 is covalently bound to an Ida linker forming a homodimer of residues 1 through 15.
XRRRRQAVRIPIPFK

SEQ ID NO: 301 - protein, 15 aa, Artificial Sequence
  Synthetic
  MISC_FEATURE (1)..(1): Xaa = Acylated Arginine
  MISC_FEATURE (15)..(15): The side chain of Lysine at position 15 is covalently bound to an Ida linker forming a homodimer of residues 1 through 15.
XRRRRQAVRITITFK

SEQ ID NO: 302 - protein, 15 aa, Artificial Sequence
  Synthetic
  MISC_FEATURE (1)..(1): Xaa = Acylated Arginine
  MISC_FEATURE (15)..(15): The side chain of Lysine at position 15 is covalently bound to an Ida linker forming a homodimer of residues 1 through 15.
XRRRRQAVRIAIAFK

SEQ ID NO: 303 - protein, 15 aa, Artificial Sequence
  Synthetic
  MISC_FEATURE (1)..(1): Xaa = Acylated Arginine
  MISC_FEATURE (15)..(15): The side chain of Lysine at position 15 is covalently bound to an Ida linker forming a homodimer of residues 1 through 15.
XRRRRQAVRIQIQFK

SEQ ID NO: 304 - protein, 15 aa, Artificial Sequence
  Synthetic
  MISC_FEATURE (1)..(1): Xaa = Acylated Arginine
  MISC_FEATURE (15)..(15): The side chain of Lysine at position 15 is covalently bound to an Ida linker forming a homodimer of residues 1 through 15.
XRRRRQAVRIDIDFK

SEQ ID NO: 305 - protein, 14 aa, Artificial Sequence
  Synthetic
  MISC_FEATURE (1)..(1): Xaa = Acylated Arginine
  MISC_FEATURE (12)..(12): The side chain of Lysine at position 12 is covalently bound to an Ida linker forming a homodimer of residues 1 through 14.
XRRRRQAVRIIKVN

SEQ ID NO: 306 - protein, 13 aa, Artificial Sequence
  Synthetic
  MISC_FEATURE (1)..(1): Xaa = Acylated Arginine
  MISC_FEATURE (11)..(11): The side chain of Lysine at position 11 is covalently bound to an Ida linker forming a homodimer of residues 1 through 13.
XRRRRQAVRIKFV

SEQ ID NO: 307 - protein, 13 aa, Artificial Sequence
  Synthetic
  MISC_FEATURE (1)..(1): Xaa = Acylated Arginine
  MISC_FEATURE (10)..(10): The side chain of Lysine at position 10 is covalently bound to an Ida linker forming a homodimer of residues 1 through 13.
XRRRRQAVRKIFV

SEQ ID NO: 308 - protein, 13 aa, Artificial Sequence
  Synthetic
  MISC_FEATURE (1)..(1): Xaa = Acylated Arginine
  MISC_FEATURE (9)..(9): The side chain of Lysine at position 9 is covalently bound to an Ida linker forming a homodimer of residues 1 through 13.
XRRRRQAVKIIFV

SEQ ID NO: 309 - protein, 13 aa, Artificial Sequence
  Synthetic
  MISC_FEATURE (1)..(1): Xaa = Acylated Arginine
  MISC_FEATURE (8)..(8): The side chain of Lysine at position 8 is covalently bound to an Ida linker forming a homodimer of residues 1 through 13.
XRRRRQAKRIIFV

SEQ ID NO: 310 - protein, 13 aa, Artificial Sequence
  Synthetic
  MISC_FEATURE (1)..(1): Xaa = Acylated Arginine
  MISC_FEATURE (7)..(7): `The side chain of Lysine at position 7 is covalently bound to an Ida linker forming a homodimer of residues 1 through 13.
XRRRRQKVRIIFV

SEQ ID NO: 311 - protein, 15 aa, Artificial Sequence
  Synthetic
  MISC_FEATURE (1)..(1): Xaa = Acylated Arginine
XRRRRQAVRITITFK

SEQ ID NO: 312 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XARRRQAVAIEFK

SEQ ID NO: 313 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XARRRQAVAIGFK

SEQ ID NO: 314 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XARRRQAVAIPFK

SEQ ID NO: 315 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVAIYFK

SEQ ID NO: 316 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVHIYFK

SEQ ID NO: 317 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQHVAIYFK

SEQ ID NO: 318 - protein, 13 aa, Artificial Sequence
  Synthetic
  MISC_FEATURE (1)..(1): Xaa = Acylated Histidine
  MISC_FEATURE (13)..(13): The side chain of Lysine at position 13 is covalently bound to an Ida linker forming a homodimer of residues 1 through 13.
XRRRRQAVAIYFK

SEQ ID NO: 319 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQHVHIYFK

SEQ ID NO: 320 - protein, 13 aa, Artificial Sequence
  Synthetic
  MISC_FEATURE (1)..(1): Xaa = Acylated Histidine
  MISC_FEATURE (13)..(13): The side chain of Lysine at position 13 is covalently bound to an Ida linker forming a homodimer of residues 1 through 13.
XRRRRQAVHIYFK

SEQ ID NO: 321 - protein, 13 aa, Artificial Sequence
  Synthetic
  MISC_FEATURE (1)..(1): Xaa = Acylated Histidine
  MISC_FEATURE (13)..(13): The side chain of Lysine at position 13 is covalently bound to an Ida linker forming a homodimer of residues 1 through 13.
XRRRRQHVAIYFK

SEQ ID NO: 322 - protein, 13 aa, Artificial Sequence
  Synthetic
  MISC_FEATURE (1)..(1): Xaa = Acylated Histidine
  MISC_FEATURE (13)..(13): The side chain of Lysine at position 13 is covalently bound to an Ida linker forming a homodimer of residues 1 through 13.
XRRRRQHVHIYFK

SEQ ID NO: 323 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XHRRRQHVRIYFK

SEQ ID NO: 324 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRHRRQHVRIYFK

SEQ ID NO: 325 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRHRQHVRIYFK

SEQ ID NO: 326 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRHQHVRIYFK

SEQ ID NO: 327 - protein, 13 aa, Artificial Sequence
  Synthetic
  MISC_FEATURE (1)..(1): Xaa = Acylated Histidine
  MISC_FEATURE (13)..(13): The side chain of Lysine at position 13 is covalently bound to an Ida linker forming a homodimer of residues 1 through 13.
XHRRRQHVRIYFK

SEQ ID NO: 328 - protein, 13 aa, Artificial Sequence
  Synthetic
  MISC_FEATURE (1)..(1): Xaa = Acylated Histidine
  MISC_FEATURE (13)..(13): The side chain of Lysine at position 13 is covalently bound to an Ida linker forming a homodimer of residues 1 through 13.
XRHRRQHVRIYFK

SEQ ID NO: 329 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an GP3 linker forming a homodimer of residues 1 through 13.
XRRRRQRVAIYFK

SEQ ID NO: 330 - protein, 13 aa, Artificial Sequence
  Synthetic
  MISC_FEATURE (1)..(1): Xaa = Acylated Histidine
  MISC_FEATURE (13)..(13): The side chain of Lysine at position 13 is covalently bound to an Ida linker forming a homodimer of residues 1 through 13.
XRRHRQHVRIYFK

SEQ ID NO: 331 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an GP3 linker forming a homodimer of residues 1 through 13.
XRRRRQRVAIYFK

SEQ ID NO: 332 - protein, 13 aa, Artificial Sequence
  Synthetic
  MISC_FEATURE (1)..(1): Xaa = Acylated Histidine
  MISC_FEATURE (13)..(13): The side chain of Lysine at position 13 is covalently bound to an Ida linker forming a homodimer of residues 1 through 13.
XRRRHQHVRIYFK

SEQ ID NO: 333 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XHHRRQHVRIYFK

SEQ ID NO: 334 - protein, 13 aa, Artificial Sequence
  Synthetic
  MISC_FEATURE (1)..(13): Peptide counterion = acetic acid
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIYFK

SEQ ID NO: 335 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XHRRHQHVRIYFK

SEQ ID NO: 336 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida PEG (20kDa) linker forming a homodimer of residues 1 through 13.
XRRRRQRVAIYFK

SEQ ID NO: 337 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XHRHRQHVRIYFK

SEQ ID NO: 338 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XHRRRQHVHIYFK

SEQ ID NO: 339 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRHHRQHVRIYFK

SEQ ID NO: 340 - protein, 13 aa, Artificial Sequence
  Synthetic
  MISC_FEATURE (1)..(1): Xaa = Acylated Arginine
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRHRHQHVRIYFK

SEQ ID NO: 341 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRHRHQRVHIYFK

SEQ ID NO: 342 - protein, 13 aa, Artificial Sequence
  Synthetic
  MISC_FEATURE (1)..(1): Xaa = Acylated Alanine
  MISC_FEATURE (12)..(12): Xaa = Homophenylalanine
  MISC_FEATURE (13)..(13): The side chain of Lysine at position 13 is covalently bound to an Ida linker forming a homodimer of residues 1 through 13.
  MISC_FEATURE (14)..(14): The side chain of Lysine at position 14 is covalently bound to an Ida linker forming a homodimer of residues 1 through 14.
XRRRRQRVAIYXK

SEQ ID NO: 343 - protein, 13 aa, Artificial Sequence
  Synthetic
  MISC_FEATURE (1)..(1): Xaa = Acylated Alanine
  MISC_FEATURE (12)..(12): Xaa = Phenylglycine
  MISC_FEATURE (13)..(13): The side chain of Lysine at position 13 is covalently bound to an Ida linker forming a homodimer of residues 1 through 13.
XRRRRQRVAIYXK

SEQ ID NO: 344 - protein, 13 aa, Artificial Sequence
  Synthetic
  MISC_FEATURE (1)..(1): Xaa = Acylated Alanine
  MISC_FEATURE (12)..(12): Xaa = Beta-Cyclohexylalanine
  MISC_FEATURE (13)..(13): The side chain of Lysine at position 13 is covalently bound to an Ida linker forming a homodimer of residues 1 through 13.
XRRRRQRVAIYXK

SEQ ID NO: 345 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIYWK

SEQ ID NO: 346 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIYEK

SEQ ID NO: 347 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIYTK

SEQ ID NO: 348 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIYHK

SEQ ID NO: 349 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIYPK

SEQ ID NO: 350 - protein, 13 aa, Artificial Sequence
  Synthetic
  MISC_FEATURE (1)..(1): Xaa = Acylated Alanine
  MISC_FEATURE (12)..(12): Xaa = diphenylalanine
  MISC_FEATURE (13)..(13): The side chain of Lysine at position 13 is covalently bound to an Ida linker forming a homodimer of residues 1 through 13.
XRRRRQRVAIYXK

SEQ ID NO: 351 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIYGK

SEQ ID NO: 352 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIYIK

SEQ ID NO: 353 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIYYK

SEQ ID NO: 354 - protein, 13 aa, Artificial Sequence
  Synthetic
  MISC_FEATURE (1)..(1): Xaa = Acylated Alanine
  MISC_FEATURE (11)..(11): Xaa = the “D” enantiomer of Ile
XRRRRQRVAIXFK

SEQ ID NO: 355 - protein, 13 aa, Artificial Sequence
  Synthetic
  MISC_FEATURE (1)..(1): Xaa = Acylated Alanine
  MISC_FEATURE (6)..(6): Xaa = the “D” enantiomer of Gln
XRRRRXRVAIIFK

SEQ ID NO: 356 - protein, 13 aa, Artificial Sequence
  Synthetic
  MISC_FEATURE (1)..(1): Xaa = Acylated Alanine
  MISC_FEATURE (2)..(2): Xaa = the “D” enantiomer of Arg
XXRRRQRVAIIFK

SEQ ID NO: 357 - protein, 15 aa, Artificial Sequence
  Synthetic
  MISC_FEATURE (1)..(1): Xaa = Acylated Arginine
XRRRRQAVRIQIQFK

SEQ ID NO: 358 - protein, 15 aa, Artificial Sequence
  Synthetic
  MISC_FEATURE (1)..(1): Xaa = Acylated Arginine
XRRRRQAVRIDIDFK

SEQ ID NO: 359 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRHHQHVRIYFK

SEQ ID NO: 360 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRHRQHVHIYFK

SEQ ID NO: 361 - protein, 13 aa, Artificial Sequence
  Synthetic
  MISC_FEATURE (1)..(1): Xaa = Acylated Alanine
XRRRRQRVAIYFK

SEQ ID NO: 362 - protein, 13 aa, Artificial Sequence
  Synthetic
  MISC_FEATURE (1)..(1): Xaa = Acylated Alanine
  MISC_FEATURE (10)..(10): Xaa = Beta-Cyclohexylalanine
  MISC_FEATURE (13)..(13): The side chain of Lysine at position 13 is covalently bound to an Ida linker forming a homodimer of residues 1 through 13.
XRRRRQRVAXYFK

SEQ ID NO: 363 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAWYFK

SEQ ID NO: 364 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAEYFK

SEQ ID NO: 365 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVATYFK

SEQ ID NO: 366 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAHYFK

SEQ ID NO: 367 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAPYFK

SEQ ID NO: 368 - protein, 13 aa, Artificial Sequence
  Synthetic
  MISC_FEATURE (1)..(1): Xaa = Acylated Alanine
  MISC_FEATURE (10)..(10): Xaa = 3-diphenylalanine
  MISC_FEATURE (13)..(13): The side chain of Lysine at position 13 is covalently bound to an Ida linker forming a homodimer of residues 1 through 13.
XRRRRQRVAXYFK

SEQ ID NO: 369 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAQYFK

SEQ ID NO: 370 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAFYFK

SEQ ID NO: 371 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAYYFK

SEQ ID NO: 372 - protein, 13 aa, Artificial Sequence
  Synthetic
  MISC_FEATURE (1)..(1): Xaa = Acylated Alanine
  MISC_FEATURE (10)..(10): Xaa = Homophenylalanine
  MISC_FEATURE (13)..(13): The side chain of Lysine at position 13 is covalently bound to an Ida linker forming a homodimer of residues 1 through 13.
XRRRRQRVAXYFK

SEQ ID NO: 373 - protein, 13 aa, Artificial Sequence
  Synthetic
  MISC_FEATURE (1)..(1): Xaa = Acylated Alanine
  MISC_FEATURE (10)..(10): Xaa = Homophenylalanine
  MISC_FEATURE (10)..(10): Xaa = the “D” enantiomer of Homophenylalanine
  MISC_FEATURE (13)..(13): The side chain of Lysine at position 13 is covalently bound to an Ida linker forming a homodimer of residues 1 through 13.
XRRRRQRVAXYFK

SEQ ID NO: 374 - protein, 13 aa, Artificial Sequence
  Synthetic
  MISC_FEATURE (1)..(1): Xaa = Acylated Alanine
  MISC_FEATURE (10)..(10): Xaa = Phenylglycine
  MISC_FEATURE (13)..(13): The side chain of Lysine at position 13 is covalently bound to an Ida linker forming a homodimer of residues 1 through 13.
XRRRRQRVAXYFK

SEQ ID NO: 375 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida PEG (30 kDa) linker forming a homodimer of residues 1 through 13.
XRRRRQRVRIIFK

SEQ ID NO: 376 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida PEG (30 kDa) linker forming a homodimer of residues 1 through 13.
XRRRRQRVAIYFK

SEQ ID NO: 377 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIMFK

SEQ ID NO: 378 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIQFK

SEQ ID NO: 379 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAISFK

SEQ ID NO: 380 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQEVAIYFK

SEQ ID NO: 381 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida PEG (30 kDa) linker forming a homodimer of residues 1 through 13.
XRRRRQAVAIYFK

SEQ ID NO: 382 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQRVAIVFK

SEQ ID NO: 383 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQFVAIYFK

SEQ ID NO: 384 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQGVAIYFK

SEQ ID NO: 385 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQMVAIYFK

SEQ ID NO: 386 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQNVAIYFK

SEQ ID NO: 387 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVEIYFK

SEQ ID NO: 388 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVFIYFK

SEQ ID NO: 389 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVGIYFK

SEQ ID NO: 390 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVMIYFK

SEQ ID NO: 391 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVNIYFK

SEQ ID NO: 392 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVPIYFK

SEQ ID NO: 393 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVQIYFK

SEQ ID NO: 394 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVSIYFK

SEQ ID NO: 395 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVTIYFK

SEQ ID NO: 396 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVWIYFK

SEQ ID NO: 397 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVYIYFK

SEQ ID NO: 398 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVLIYFK

SEQ ID NO: 399 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an ATA linker forming a homotrimer
XRRRRQAVRIYFK

SEQ ID NO: 400 - protein, 13 aa, Artificial Sequence
  Synthetic
  MISC_FEATURE (1)..(1): Xaa = Acylated Aspartate
  MISC_FEATURE (13)..(13): The side chain of Lysine at position 13 is covalently bound to an Ida linker forming a homodimer of residues 1 through 13.
XRRRRQAVAIYFK

SEQ ID NO: 401 - protein, 13 aa, Artificial Sequence
  Synthetic
  MISC_FEATURE (1)..(1): Xaa = Acylated Glutamate
  MISC_FEATURE (13)..(13): The side chain of Lysine at position 13 is covalently bound to an Ida linker forming a homodimer of residues 1 through 13.
XRRRRQAVAIYFK

SEQ ID NO: 402 - protein, 13 aa, Artificial Sequence
  Synthetic
  MISC_FEATURE (1)..(1): Xaa = Acylated Phenylalanine
  MISC_FEATURE (13)..(13): The side chain of Lysine at position 13 is covalently bound to an Ida linker forming a homodimer of residues 1 through 13.
XRRRRQAVAIYFK

SEQ ID NO: 403 - protein, 13 aa, Artificial Sequence
  Synthetic
  MISC_FEATURE (1)..(1): Xaa = Acylated Glycine
  MISC_FEATURE (13)..(13): The side chain of Lysine at position 13 is covalently bound to an Ida linker forming a homodimer of residues 1 through 13.
XRRRRQAVAIYFK

SEQ ID NO: 404 - protein, 13 aa, Artificial Sequence
  Synthetic
  MISC_FEATURE (1)..(1): Xaa = Acylated Isoleucine
  MISC_FEATURE (13)..(13): The side chain of Lysine at position 13 is covalently bound to an Ida linker forming a homodimer of residues 1 through 13.
XRRRRQAVAIYFK

SEQ ID NO: 405 - protein, 13 aa, Artificial Sequence
  Synthetic
  MISC_FEATURE (1)..(1): Xaa = Acylated Leucine
  MISC_FEATURE (13)..(13): The side chain of Lysine at position 13 is covalently bound to an Ida linker forming a homodimer of residues 1 through 13.
XRRRRQAVAIYFK

SEQ ID NO: 406 - protein, 13 aa, Artificial Sequence
  Synthetic
  MISC_FEATURE (1)..(1): Xaa = Acylated Methionine
  MISC_FEATURE (13)..(13): The side chain of Lysine at position 13 is covalently bound to an Ida linker forming a homodimer of residues 1 through 13.
XRRRRQAVAIYFK

SEQ ID NO: 407 - protein, 13 aa, Artificial Sequence
  Synthetic
  MISC_FEATURE (1)..(1): Xaa = Acylated Asparagine
  MISC_FEATURE (13)..(13): The side chain of Lysine at position 13 is covalently bound to an Ida linker forming a homodimer of residues 1 through 13.
XRRRRQAVAIYFK

SEQ ID NO: 408 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRRQAVAIYFK

SEQ ID NO: 409 - protein, 13 aa, Artificial Sequence
  Synthetic
  MISC_FEATURE (1)..(1): Xaa = Acylated Glutamine
  MISC_FEATURE (13)..(13): The side chain of Lysine at position 13 is covalently bound to an Ida linker forming a homodimer of residues 1 through 13.
XRRRRQAVAIYFK

SEQ ID NO: 410 - protein, 13 aa, Artificial Sequence
  Synthetic
  MISC_FEATURE (1)..(1): Xaa = Acylated Serine
  MISC_FEATURE (13)..(13): The side chain of Lysine at position 13 is covalently bound to an Ida linker forming a homodimer of residues 1 through 13.
XRRRRQAVAIYFK

SEQ ID NO: 411 - protein, 13 aa, Artificial Sequence
  Synthetic
  MISC_FEATURE (1)..(1): Xaa = Acylated Threonine
  MISC_FEATURE (13)..(13): The side chain of Lysine at position 13 is covalently bound to an Ida linker forming a homodimer of residues 1 through 13.
XRRRRQAVAIYFK

SEQ ID NO: 412 - protein, 13 aa, Artificial Sequence
  Synthetic
  MISC_FEATURE (1)..(1): Xaa = Acylated Valine
  MISC_FEATURE (13)..(13): The side chain of Lysine at position 13 is covalently bound to an Ida linker forming a homodimer of residues 1 through 13.
XRRRRQAVAIYFK

SEQ ID NO: 413 - protein, 13 aa, Artificial Sequence
  Synthetic
  MISC_FEATURE (1)..(1): Xaa = Acylated Tryptophan
  MISC_FEATURE (13)..(13): The side chain of Lysine at position 13 is covalently bound to an Ida linker forming a homodimer of residues 1 through 13.
XRRRRQAVAIYFK

SEQ ID NO: 414 - protein, 13 aa, Artificial Sequence
  Synthetic
  MISC_FEATURE (1)..(1): Xaa = Acylated Tyrosine
  MISC_FEATURE (13)..(13): The side chain of Lysine at position 13 is covalently bound to an Ida linker forming a homodimer of residues 1 through 13.
XRRRRQAVAIYFK

SEQ ID NO: 415 - protein, 13 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 13.
XRRRRQAVRIKFV

SEQ ID NO: 416 - protein, 12 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 12.
XRRRRQAVRIKF

SEQ ID NO: 417 - protein, 13 aa, Artificial Sequence
  Synthetic
  MISC_FEATURE (1)..(1): Xaa = Acylated Arginine
  MISC_FEATURE (11)..(11): The side chain of Lysine at position 11 is covalently bound to an Ida linker forming a homodimer of residues 1 through 13.
XRRRRQAVAIKFV

SEQ ID NO: 418 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQPVAIYFK

SEQ ID NO: 419 - protein, 13 aa, Artificial Sequence
  Synthetic
  MISC_FEATURE (1)..(1): Xaa = Acylated Alanine
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQQVAIYFK

SEQ ID NO: 420 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQSVAIYFK

SEQ ID NO: 421 - protein, 12 aa, Artificial Sequence
  Synthetic
  MISC_FEATURE (1)..(1): Xaa = Acylated Arginine
  MISC_FEATURE (11)..(11): The side chain of Lysine at position 11 is covalently bound to an Ida linker forming a homodimer of residues 1 through 12.
XRRRRQAVAIKF

SEQ ID NO: 422 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQTVAIYFK

SEQ ID NO: 423 - protein, 13 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 13.
XRRRRQRVAIKFV

SEQ ID NO: 424 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQWVAIYFK

SEQ ID NO: 425 - protein, 12 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 12.
XRRRRQRVAIKF

SEQ ID NO: 426 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQYVAIYFK

SEQ ID NO: 427 - protein, 12 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 12.
XRRRRQAVAIKF

SEQ ID NO: 428 - protein, 12 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an GP3 linker forming a homodimer of residues 1 through 12.
XRRRRQAVAIKF

SEQ ID NO: 429 - protein, 13 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 13.
XRRRRQAVAIKFI

SEQ ID NO: 430 - protein, 13 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 13.
XRRRRQAVAIKYF

SEQ ID NO: 431 - protein, 13 aa, Artificial Sequence
  Synthetic
  MISC_FEATURE (1)..(1): Xaa = Acylated Alanine
  MISC_FEATURE (12)..(12): The side chain of Lysine at position 12 is covalently bound to an Ida linker forming a homodimer of residues 1 through 13.
XRRRRQAVAIYKF

SEQ ID NO: 432 - skipped

SEQ ID NO: 433 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRREQAVAIYFK

SEQ ID NO: 434 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRFQAVAIYFK

SEQ ID NO: 435 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRGQAVAIYFK

SEQ ID NO: 436 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRLQAVAIYFK

SEQ ID NO: 437 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRMQAVAIYFK

SEQ ID NO: 438 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRNQAVAIYFK

SEQ ID NO: 439 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRPQAVAIYFK

SEQ ID NO: 440 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRSQAVAIYFK

SEQ ID NO: 441 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRTQAVAIYFK

SEQ ID NO: 442 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRVQAVAIYFK

SEQ ID NO: 443 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRWQAVAIYFK

SEQ ID NO: 444 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRYQAVAIYFK

SEQ ID NO: 445 - protein, 11 aa, Artificial Sequence
  Synthetic
YGRKKRRQRRR

SEQ ID NO: 446 - protein, 13 aa, Artificial Sequence
  Synthetic
  MISC_FEATURE (1)..(1): Xaa = Acylated Arginine
  MISC_FEATURE (13)..(13): The side chain of Lys at position 13 is covalently bound to an ATA linker forming a homotrimer
XRRRRQAVAIYFK

SEQ ID NO: 447 - protein, 13 aa, Artificial Sequence
  Synthetic
  MISC_FEATURE (1)..(1): Xaa = Acylated Alanine
  MISC_FEATURE (11)..(11): Xaa = the “D” enantiomer of Ile
  MISC_FEATURE (13)..(13): The side chain of Lysine at position 13 is covalently lbound to an ATA linker forming a homotrimer
XRRRRQAVRIXFK

SEQ ID NO: 448 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRRQAAAAAAK

SEQ ID NO: 449 - protein, 13 aa, Artificial Sequence
  Synthetic
  MISC_FEATURE (1)..(1): Xaa = Acylated Arginine
  MISC_FEATURE (13)..(13): The side chain of Lysine at position 13 is covalently bound to an Ida linker forming a homodimer of residues 1 through 13.
XRRRRAAAAAAAK

SEQ ID NO: 450 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRQAVAILFK

SEQ ID NO: 451 - protein, 13 aa, Artificial Sequence
  Synthetic
  MISC_FEATURE (1)..(1): Xaa = Acylated Asparagine
  MISC_FEATURE (13)..(13)
XRRRRQAVAIVFK

SEQ ID NO: 452 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRRQAVAIVFK

SEQ ID NO: 453 - protein, 13 aa, Artificial Sequence
  Synthetic
  MISC_FEATURE (1)..(1): Xaa = Acylated Glutamine
  MISC_FEATURE (13)..(13): The side chain of Lysine at position 13 is covalently bound to an Ida linker forming a homodimer of residues 1 through 13.
XRRRRQAVAIVFK

SEQ ID NO: 454 - protein, 13 aa, Artificial Sequence
  Synthetic
  MISC_FEATURE (1)..(1): Xaa = Acylated Serine
  MISC_FEATURE (13)..(13): The side chain of Lysine at position 13 is covalently bound to an Ida linker forming a homodimer of residues 1 through 13.
XRRRRQAVAIVFK

SEQ ID NO: 455 - protein, 13 aa, Artificial Sequence
  Synthetic
  MISC_FEATURE (1)..(1): Xaa = Acylated Threonine
  MISC_FEATURE (13)..(13): The side chain of Lysine at position 13 is covalently bound to an Ida linker forming a homodimer of residues 1 through 13.
XRRRRQAVAIVFK

SEQ ID NO: 456 - protein, 12 aa, Artificial Sequence
  Synthetic
  MISC_FEATURE (1)..(1): Xaa = Acylated Lysine
  MISC_FEATURE (1)..(1): The side chain of Lysine at position 1 is covalently bound to an Ida linker forming a homodimer of residues 1 through 13.
XRRRRQAVAIVF

SEQ ID NO: 457 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an ATA linker forming a homotrimer
XRRRRQAVAIYFK

SEQ ID NO: 458 - protein, 13 aa, Artificial Sequence
  Synthetic
  MISC_FEATURE (1)..(1): Xaa = Acylated Lysine
  MISC_FEATURE (1)..(1): The side chain of Lysine at position 1 is covalently bound to an Ida linker forming a homodimer of residues 1 through 13.
XARRRRQAVAIYF

SEQ ID NO: 459 - protein, 12 aa, Artificial Sequence
  Synthetic
  MISC_FEATURE (1)..(1): Xaa = Acylated Lysine
  MISC_FEATURE (1)..(1): The side chain of Lysine at position 1 is covalently bound to an Ida linker forming a homodimer of residues 1 through 12.
XRRRRQAVAIYF

SEQ ID NO: 460 - protein, 12 aa, Artificial Sequence
  Synthetic
  MISC_FEATURE (1)..(1): Xaa = Acylated Alanine
  MISC_FEATURE (4)..(4): The side chain of Lysine at position 4 is covalently bound to an Ida linker forming a homodimer of residues 1 through 12.
XRRKRQAVAIYF

SEQ ID NO: 461 - protein, 12 aa, Artificial Sequence
  Synthetic
  MISC_FEATURE (1)..(1): Xaa = Acylated Alanine
  MISC_FEATURE (5)..(5): The side chain of Lysine at position 5 is covalently bound to an Ida linker forming a homodimer of residues 1 through 12.
XRRRKQAVAIYF

SEQ ID NO: 462 - protein, 12 aa, Artificial Sequence
  Synthetic
  MISC_FEATURE (1)..(1): Xaa = Acylated Alanine
  MISC_FEATURE (6)..(6): The side chain of Lysine at position 6 is covalently bound to an Ida linker forming a homodimer of residues 1 through 12.
XRRRRKAVAIYF

SEQ ID NO: 463 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRARQAVAIYFK

SEQ ID NO: 464 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRERQAVAIYFK

SEQ ID NO: 465 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRQRQAVAIYFK

SEQ ID NO: 466 - protein, 12 aa, Artificial Sequence
  Synthetic
  MISC_FEATURE (1)..(1): Xaa = Acylated Alanine
  MISC_FEATURE (2)..(2): The side chain of Lysine at position 2 is covalently bound to an Ida linker forming a homodimer of residues 1 through 12.
XKRRRQAVAIYF

SEQ ID NO: 467 - protein, 12 aa, Artificial Sequence
  Synthetic
  MISC_FEATURE (1)..(1): Xaa = Acylated Alanine
  MISC_FEATURE (3)..(3): The side chain of Lysine at position 3 is covalently bound to an Ida linker forming a homodimer of residues 1 through 12.
XRKRRQAVAIYF

SEQ ID NO: 468 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRIRQAVAIYFK

SEQ ID NO: 469 - protein, 26 aa, Artificial Sequence
  Synthetic
  MISC_FEATURE (1)..(1): Xaa = Acylated Alanine
XRRRRQRVAIYFKARRRRQRVAIYFK

SEQ ID NO: 470 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRDQAVAIYFK

SEQ ID NO: 471 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRFQAVAIYFK

SEQ ID NO: 472 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRGQAVAIYFK

SEQ ID NO: 473 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRLQAVAIYFK

SEQ ID NO: 474 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRMQAVAIYFK

SEQ ID NO: 475 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRNQAVAIYFK

SEQ ID NO: 476 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRPQAVAIYFK

SEQ ID NO: 477 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRSQAVAIYFK

SEQ ID NO: 478 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRTQAVAIYFK

SEQ ID NO: 479 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRVQAVAIYFK

SEQ ID NO: 480 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRWQAVAIYFK

SEQ ID NO: 481 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRRYQAVAIYFK

SEQ ID NO: 482 - protein, 12 aa, Artificial Sequence
  Synthetic
  MISC_FEATURE (1)..(1): Xaa = Acylated Proline
  MISC_FEATURE (11)..(11): The side chain of Lysine at position 11 is covalently bound to an Ida linker forming a homodimer of residues 1 through 12.
XRRRRQAVAIKF

SEQ ID NO: 483 - protein, 12 aa, Artificial Sequence
  Synthetic
  MISC_FEATURE (1)..(1): Xaa = Acylated Gln
  MISC_FEATURE (11)..(11): The side chain of Lysine at position 11 is covalently bound to an Ida linker forming a homodimer of residues 1 through 12.
XRRRRQAVAIKF

SEQ ID NO: 484 - protein, 12 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 12.
XRRRNQAVAIKF

SEQ ID NO: 485 - protein, 12 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 12.
XRRRPQAVAIKF

SEQ ID NO: 486 - protein, 12 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 12.
XRRRRQSVAIKF

SEQ ID NO: 487 - protein, 12 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 12.
XRRRRQPVAIKF

SEQ ID NO: 488 - protein, 12 aa, Artificial Sequence
  Synthetic
  MISC_FEATURE (1)..(1): Xaa = Acylated Proline
  MISC_FEATURE (11)..(11): The side chain of Lysine at position 11 is covalently bound to an Ida linker forming a homodimer of residues 1 through 12.
XRRRNQAVAIKF

SEQ ID NO: 489 - protein, 12 aa, Artificial Sequence
  Synthetic
  MISC_FEATURE (1)..(1): Xaa = Acylated Proline
  MISC_FEATURE (11)..(11): The side chain of Lysine at position 11 is covalently bound to an Ida linker forming a homodimer of residues 1 through 12.
XRRRPQAVAIKF

SEQ ID NO: 490 - protein, 12 aa, Artificial Sequence
  Synthetic
  MISC_FEATURE (1)..(1): Xaa = Acylated Proline
  MISC_FEATURE (11)..(11): The side chain of Lysine at position 11 is covalently bound to an Ida linker forming a homodimer of residues 1 through 12.
XRRRRQSVAIKF

SEQ ID NO: 491 - protein, 12 aa, Artificial Sequence
  Synthetic
  MISC_FEATURE (1)..(1): Xaa = Acylated Proline
  MISC_FEATURE (11)..(11): The side chain of Lysine at position 11 is covalently bound to an Ida linker forming a homodimer of residues 1 through 12.
XRRRRQPVAIKF

SEQ ID NO: 492 - protein, 12 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 12.
XRRRNQSVAIKF

SEQ ID NO: 493 - protein, 12 aa, Artificial Sequence
  Synthetic
  MISC_FEATURE (1)..(1): Xaa = Acylated Alanine
  MISC_FEATURE (11)..(11): The side chain of Lysine at position 11 is covalently bound to an Ida linker forming a homodimer of residues 1 through 12.
XRRRNQPVAIKF

SEQ ID NO: 494 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRNRRQAVAIYFK

SEQ ID NO: 495 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRERRQAVAIYFK

SEQ ID NO: 496 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRHRRQAVAIYFK

SEQ ID NO: 497 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRLRRQAVAIYFK

SEQ ID NO: 498 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRSRRQAVAIYFK

SEQ ID NO: 499 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRYRRQAVAIYFK

SEQ ID NO: 500 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRNRQAVAIYFK

SEQ ID NO: 501 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRERQAVAIYFK

SEQ ID NO: 502 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRHRQAVAIYFK

SEQ ID NO: 503 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRLRQAVAIYFK

SEQ ID NO: 504 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRSRQAVAIYFK

SEQ ID NO: 505 - protein, 13 aa, Artificial Sequence
  Synthetic
  MISC_FEATURE (1)..(1): Xaa = Acylated Proline
  MISC_FEATURE (13)..(13): The side chain of Lysine at position 13 is covalently bound to an Ida linker forming a homodimer of residues 1 through 13.
XRRYRQAVAIYFK

SEQ ID NO: 506 - protein, 20 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 20.
XRRRRQRVAIYFKGGRGDSP

SEQ ID NO: 507 - protein, 20 aa, Artificial Sequence
  Synthetic
  MISC_FEATURE (1)..(1): Xaa = Acylated Arginine
  MISC_FEATURE (20)..(20): The side chain of Lysine at position 20 is covalently bound to an Ida linker forming a homodimer of residues 1 through 20.
XGDSPGGARRRRQRVAIYFK

SEQ ID NO: 508 - protein, 20 aa, Artificial Sequence
  Synthetic
  MISC_FEATURE (1)..(1): Xaa = Acylated Cystine
  MISC_FEATURE (20)..(20): The side chain of Lysine at position 20 is covalently bound to an Ida linker forming a homodimer of residues 1 through 20.
XNGRCGGARRRRQRVAIYFK

SEQ ID NO: 509 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRARVAIYFK

SEQ ID NO: 510 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRERVAIYFK

SEQ ID NO: 511 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRFRVAIYFK

SEQ ID NO: 512 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRGRVAIYFK

SEQ ID NO: 513 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRIRVAIYFK

SEQ ID NO: 514 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRMRVAIYFK

SEQ ID NO: 515 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRNRVAIYFK

SEQ ID NO: 516 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRPRVAIYFK

SEQ ID NO: 517 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRTRVAIYFK

SEQ ID NO: 518 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRVRVAIYFK

SEQ ID NO: 519 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRWRVAIYFK

SEQ ID NO: 520 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an Ida linker forming a homodimer of residues 1 through 13.
XRRRRYRVAIYFK

SEQ ID NO: 521 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an DSS linker forming a homodimer of residues 1 through 13.
XRRRRQRVAIYFK

SEQ ID NO: 522 - protein, 13 aa, Artificial Sequence
  Synthetic
  MISC_FEATURE (1)..(1): Xaa = Acylated Alanine
  MISC_FEATURE (13)..(13): The side chain of Lysine at position 13 is covalently bound to an DST linker forming a homodimer of residues 1 through 13.
XRRRRQRVAIYFK

SEQ ID NO: 523 - protein, 21 aa, Artificial Sequence
  Synthetic
  MISC_FEATURE (1)..(1): Xaa = Acylated Alanine
  MISC_FEATURE (13)..(14): Lysine at position 13 is linked with an Ida linker to acylated Lysine at position 14.  The carboxy terminus of Lysine at position 13 is not bound to the amino terminus of the acylated Lysine at position 14.
  MISC_FEATURE (14)..(14): Xaa = Acylated Lysine
XRRRRQRVAIYFKXGGRGDSP

SEQ ID NO: 524 - protein, 21 aa, Artificial Sequence
  Synthetic
  MISC_FEATURE (1)..(1): Xaa = Acylated Alanine
  MISC_FEATURE (13)..(14): Lysine at position 13 is linked with a Ida-Peg (3.4 kDa) linker to acylated Lysine at position 14.  The carboxy terminus of Lysine at position 13 is not bound to the amino terminus of Acylated Lysine at position 14.
  MISC_FEATURE (14)..(14): Xaa = Acylated Lysine
XRRRRQRVAIYFKXGGRGDSP

(19) United States
(12) Patent Application Publication
(10) Pub. No.: US 2008/0090760 A2
(43) Pub. Date: Apr. 17, 2008

(54) Compositions and Methods for Inhibiting Cellular Proliferation

(76) Inventors: Todd Hembrough, Damascus, MD (US); Christopher Holmes, Saratoga, CA (US); Qun Yin, Palo Alto, CA (US); Genet Zemede, Santa Clara, CA (US); Yvonne Angell, San Jose, CA (US); Brian Frederick, Ben Lomond, CA (US); Caiding Xu, San Jose, CA (US)

Correspondence Address: KING & SPALDING LLP, 1180 PEACHTREE STREET, ATLANTA, GA, 30309-3521, US, 404-572-4600, 404-572-5134

(21) Appl. No.: 11/636,353

(22) Filed: Dec. 8, 2006

Related U.S. Application Data
(60) Provisional application No. 60/749,276, filed on Dec. 9, 2005.

(51) Int. Cl. A61K 38/10; A61K 38/08; C07K 7/08; C07K 7/06

(52) U.S. Cl. 514/12; 514/13; 514/14; 514/15; 514/16; 530/326; 530/328; 530/327; 530/329

(57) ABSTRACT

Compositions and methods effective in inhibiting abnormal or undesirable cell proliferation, particularly endothelial cell proliferation and angiogenesis related to neovascularization and tumor growth are provided. The compositions comprise peptide molecules, optionally containing one or more individual peptide chains covalently linked, and optionally modified with polyethylene glycol (PEG). The methods involve administering to a human or animal the composition described herein in a dosage sufficient to inhibit cell proliferation, particularly endothelial cell proliferation. The methods are useful for treating diseases and processes mediated by undesired and uncontrolled cell proliferation, such as cancer, particularly by inhibiting angiogenesis. Administration of the composition to a human or animal having prevascularized, metastasized tumors is useful for preventing the growth or expansion of such tumors.

### CROSS REFERENCE TO RELATED PATENT APPLICATIONS

The present application claims priority to U.S. Provisional Application Ser. No. 60/749,276, filed Dec. 9, 2005, which is incorporated herein by reference in its entirety.

### FIELD OF THE INVENTION

The present invention relates to methods and compositions for the inhibition of cellular proliferation. More particularly, the present invention relates to peptide molecules, containing one or more individual peptide chains covalently linked, and their use for inhibiting angiogenesis and angiogenesis-related diseases.

### BACKGROUND OF THE INVENTION

Angiogenesis and angiogenesis related diseases are closely affected by cellular proliferation. As used herein, the term “angiogenesis” means the generation of new blood vessels into a tissue or organ. Under normal physiological conditions, humans or animals undergo angiogenesis only in very specific restricted situations. For example, angiogenesis is normally observed in wound healing, fetal and embryonal development and formation of the corpus luteum, endometrium and placenta. The term “endothelium” is defined herein as a thin layer of flat cells that lines serous cavities, lymph vessels, and blood vessels. These cells are defined herein as “endothelial cells”. The term “endothelial inhibiting activity” means the capability of a molecule to inhibit angiogenesis in general. The inhibition of endothelial cell proliferation also results in an inhibition of angiogenesis.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

Persistent, unregulated angiogenesis occurs in a multiplicity of disease states, tumor metastasis, and abnormal growth by endothelial cells and supports the pathological damage seen in these conditions. The diverse pathological disease states in which unregulated angiogenesis is present have been grouped together as angiogenic-dependent, angiogenic-associated, or angiogenic-related diseases. These diseases are a result of abnormal or undesirable cell proliferation, particularly endothelial cell proliferation.

One example of a disease dependent on angiogenesis is ocular neovascular disease. This disease is characterized by invasion of new blood vessels into the structures of the eye, such as the retina or cornea. It is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma, and retrolental fibroplasia. Other diseases associated with corneal neovascularization include, but are not limited to, epidemic keratoconjunctivitis, Vitamin A deficiency, contact lens overwear, atopic keratitis, superior limbic keratitis, and pterygium keratitis sicca. Other diseases associated with undesirable angiogenesis include Sjögren's syndrome, acne rosacea, phylectenulosis, syphilis, Mycobacterial infections, lipid degeneration, chemical burns, bacterial ulcers, fungal ulcers, Herpes simplex infection, Herpes zoster infections, protozoan infections, Kaposi's sarcoma, Mooren's ulcer, Terrien's marginal degeneration, marginal keratolysis, rheumatoid arthritis, systemic lupus, polyarteritis, trauma, Wegener's sarcoidosis, scleritis, Stevens-Johnson's disease, pemphigoid, and radial keratotomy.

Diseases associated with neovascularization include, but are not limited to, retinal/choroidal neovascularization, diabetic retinopathy, macular degeneration, sickle cell anemia, sarcoidosis, syphilis, pseudoxanthoma elasticum, Paget's disease, vein occlusion, artery occlusion, carotid obstructive disease, chronic uveitis/vitritis, Mycobacterial infections, Lyme's disease, systemic lupus erythematosis, retinopathy of prematurity, Eales' disease, Behcet's disease, infections causing retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other eye-related diseases include, but are not limited to, diseases associated with rubeosis (neovascularization of the iris and of the angle) and diseases caused by the abnormal proliferation of fibrovascular or fibrous tissue, including all forms of prolific vitreoretinopathy.

Another angiogenesis associated disease is rheumatoid arthritis. The blood vessels in the synovial lining of the joints undergo angiogenesis. In addition to forming new vascular networks, the endothelial cells release factors and reactive oxygen species that lead to pannus growth and cartilage destruction. Angiogenesis may also play a role in osteoarthritis. The activation of the chondrocytes by angiogenic-related factors contributes to the destruction of the joint. At a later stage, the angiogenic factors promote new bone growth. Therapeutic intervention that prevents the cartilage destruction could halt the progress of the disease and provide relief for persons suffering with arthritis.

Chronic inflammation may also involve pathological angiogenesis. Such diseases as ulcerative colitis and Crohn's disease show histological changes with the ingrowth of new blood vessels into inflamed tissues. Bartonellosis, a bacterial infection found in South America, can result in a chronic stage that is characterized by proliferation of vascular endothelial cells. Another pathological role associated with angiogenesis is found in atherosclerosis. The plaques formed within the lumen of blood vessels have been shown to have angiogenic stimulatory activity.

The hypothesis that tumor growth is angiogenesis-dependent was first proposed in 1971. (Folkman, New Eng. J. Med., 285:1182-86 (1971)). In its simplest terms, this hypothesis states: “Once tumor ‘take’ has occurred, every increase in tumor cell population must be preceded by an increase in new capillaries converging on the tumor.” Tumor ‘take’ is currently understood to indicate a prevascular phase of tumor growth in which a population of tumor cells occupying a few cubic millimeters volume, and not exceeding a few million cells, can survive on existing host microvessels. Expansion of tumor volume beyond this phase requires the induction of new capillary blood vessels. For example, pulmonary micrometastases in the early prevascular phase in mice would be undetectable except by high power microscopy on histological sections.

Examples of the indirect evidence which support this concept include:

(1) The growth rate of tumors implanted in subcutaneous transparent chambers in mice is slow and linear before neovascularization, and rapid and nearly exponential after neovascularization. (Algire, et al., J. Nat. Cancer Inst., 6:73-85 (1945)).

(2) Tumors grown in isolated perfused organs where blood vessels do not proliferate are limited to 1-2 mm³ but expand rapidly to >1000 times this volume when they are transplanted to mice and become neovascularized. (Folkman, et al., Annals of Surgery, 164:491-502 (1966)).

(3) Tumor growth in the avascular cornea proceeds slowly and at a linear rate, but switches to exponential growth after neovascularization. (Gimbrone, Jr., et al., J. Nat. Cancer Inst., 52:421-27 (1974)).

(4) Tumors suspended in the aqueous fluid of the anterior chamber of a rabbit eye remain viable, avascular, and limited in size to <1 mm³. Once they are implanted on the iris vascular bed, they become neovascularized and grow rapidly, reaching 16,000 times their original volume within 2 weeks. (Gimbrone, Jr., et al., J. Exp. Med., 136:261-76).

(5) When tumors are implanted on a chick embryo chorioallantoic membrane, they grow slowly during an avascular phase of >72 hours, but do not exceed a mean diameter of 0.93+0.29 mm. Rapid tumor expansion occurs within 24 hours after the onset of neovascularization, and by day 7 these vascularized tumors reach a mean diameter of 8.0+2.5 mm. (Knighton, British J. Cancer, 35:347-56 (1977)).

(6) Vascular casts of metastases in a rabbit liver reveal heterogeneity in size of the metastases, but show a relatively uniform cut-off point for the size at which vascularization is present. Tumors are generally avascular up to 1 mm in diameter, but are neovascularized beyond that diameter. (Lien, et al., Surgery, 68:334-40 (1970)).

(7) In transgenic mice that develop carcinomas in the beta cells of the pancreatic islets, pre-vascular hyperplastic islets are limited in size to <1 mm. At 6-7 weeks of age, 4-10% of the islets become neovascularized, and from these islets arise large vascularized tumors of more than 1000 times the volume of the pre-vascular islets. (Folkman, et al., Nature, 339:58-61 (1989)).

(8) A specific antibody against VEGF (vascular endothelial growth factor) reduces microvessel density and causes “significant or dramatic” inhibition of growth of three human tumors which rely on VEGF as their sole mediator of angiogenesis (in nude mice). The antibody does not inhibit growth of the tumor cells in vitro. (Kim, et al., Nature, 362:841-44 (1993)).

(9) Anti-bFGF monoclonal antibody causes 70% inhibition of growth of a mouse tumor which is dependent upon secretion of bFGF as its only mediator of angiogenesis. The antibody does not inhibit growth of the tumor cells in vitro. (Hori, et al., Cancer Res., 51:6180-84 (1991)).

(10) Intraperitoneal injection of bFGF enhances growth of a primary tumor and its metastases by stimulating growth of capillary endothelial cells in the tumor. The tumor cells themselves lack receptors for bFGF, and bFGF is not a mitogen for the tumor cells in vitro. (Gross, et al., Proc. Am. Assoc. Cancer Res., 31:79 (1990)).

(11) A specific angiogenesis inhibitor (AGM-1470) inhibits tumor growth and metastases in vivo, but is much less active in inhibiting tumor cell proliferation in vitro. It inhibits vascular endothelial cell proliferation half-maximally at 4 logs lower concentration than it inhibits tumor cell proliferation. (Ingber, et al., Nature, 48:555-57 (1990)). There is also indirect clinical evidence that tumor growth is angiogenesis dependent.

(12) Human retinoblastomas that are metastatic to the vitreous develop into avascular spheroids that are restricted to less than 1 mm³ despite the fact that they are viable and incorporate ³H-thymidine (when removed from an enucleated eye and analyzed in vitro).

(13) Carcinoma of the ovary metastasizes to the peritoneal membrane as tiny avascular white seeds (1-3 mm³). These implants rarely grow larger until one or more of them becomes neovascularized.

(14) Intensity of neovascularization in breast cancer (Weidner, et al., New Eng. J. Med., 324:1-8 (199); Weidner, et al., J. Nat. Cancer Inst., 84:1875-87 (1992)) and in prostate cancer (Weidner, et al., Am. J. Pathol., 143(2):401-09 (1993)) correlates highly with risk of future metastasis.

(15) Metastasis from human cutaneous melanoma is rare prior to neovascularization. The onset of neovascularization leads to increased thickness of the lesion and an increased risk of metastasis. (Srivastava, et al., Am. J. Pathol., 133:419-23 (1988)).

(16) In bladder cancer, the urinary level of an angiogenic protein, bFGF, is a more sensitive indicator of status and extent of disease than is cytology. (Nguyen, et al., J. Nat. Cancer Inst., 85:241-42 (1993)).

Thus, it is clear that angiogenesis plays a major role in the metastasis of cancer. If this angiogenic activity could be repressed or eliminated, then the tumor, although present, would not grow. In the disease state, prevention of angiogenesis could avert the damage caused by the invasion of the new microvascular system. Therapies directed at control of the angiogenic processes could lead to the abrogation or mitigation of these diseases.

Angiogenesis has been associated with a number of different types of cancer, including solid tumors and blood-borne tumors. Solid tumors with which angiogenesis has been associated include, but are not limited to, rhabdomyosarcomas, retinoblastoma, Ewing's sarcoma, neuroblastoma, and osteosarcoma. Angiogenesis is also associated with blood-borne tumors, such as leukemias, any of various acute or chronic neoplastic diseases of the bone marrow in which unrestrained proliferation of white blood cells occurs, usually accompanied by anemia, impaired blood clotting, and enlargement of the lymph nodes, liver and spleen. It is believed that angiogenesis plays a role in the abnormalities in the bone marrow that give rise to leukemia tumors and multiple myeloma diseases.

One of the most frequent angiogenic diseases of childhood is the hemangioma. A hemangioma is a tumor composed of newly formed blood vessels. In most cases the tumors are benign and regress without intervention. In more severe cases, the tumors progress to large cavernous and infiltrative forms and create clinical complications. Systemic forms of hemangiomas, hemangiomatoses, have a high mortality rate. Therapy-resistant hemangiomas exist that cannot be treated with therapeutics currently in use.

Angiogenesis is also responsible for damage found in heredity diseases such as Osler-Weber-Rendu disease, or heredity hemorrhagic telangiectasia. This is an inherited disease characterized by multiple small angiomas, tumors of blood or lymph vessels. The angiomas are found in the skin and mucous membranes, often accompanied by epitaxis (nose bleeds) or gastrointestinal bleeding and sometimes with pulmonary or hepatitic arteriovenous fistula.

What is needed, therefore, is a composition and method that can inhibit angiogenesis. What is also needed is a composition and method that can inhibit the unwanted growth of blood vessels, especially in tumors.

Angiogenesis is also involved in normal physiological processes, such as reproduction and wound healing. Angiogenesis is an important step in ovulation and also in implantation of the blastula after fertilization. Prevention of angiogenesis could be used to induce amenorrhea, to block ovulation, or to prevent implantation by the blastula.

In wound healing, excessive repair or fibroplasia can be a detrimental side effect of surgical procedures and may be caused or exacerbated by angiogenesis. Adhesions are a frequent complication of surgery and lead to problems such as small bowel obstruction.

Several compounds have been used to inhibit angiogenesis. Taylor, et al. (Nature, 297:307 (1982)) have used protamine to inhibit angiogenesis. The toxicity of protamine limits its practical use as a therapeutic. Folkman, et al. (Science, 221:719 (1983), and U.S. Pat. Nos. 5,001,116 and 4,994,443) have disclosed the use of heparin and steroids to control angiogenesis. Steroids, such as tetrahydrocortisol, which lack glucocorticoid and mineralocorticoid activity, have been found to be angiogenic inhibitors.

Other factors found endogenously in animals, such as a 4 kDa glycoprotein from bovine vitreous humor and a cartilage derived factor, have been used to inhibit angiogenesis. Cellular factors, such as interferon, inhibit angiogenesis. For example, interferon alpha or human interferon beta have been shown to inhibit tumor-induced angiogenesis in mouse dermis stimulated by human neoplastic cells. Interferon beta is also a potent inhibitor of angiogenesis induced by allogeneic spleen cells. (Sidky, et al., Cancer Res., 47:5155-61(1987)). Human recombinant interferon (alpha/A) was reported to be successfully used in the treatment of pulmonary hemangiomatosis, an angiogenesis-induced disease. (White, et al., New Eng. J. Med., 320:1197-1200 (1989)).

Other agents that have been used to inhibit angiogenesis include ascorbic acid ethers and related compounds. (Japanese Kokai Tokkyo Koho No. 58-13 (1978)). Sulfated polysaccharide DS 4152 also inhibits angiogenesis. (Japanese Kokai Tokkyo Koho No. 63-119500). Additional anti-angiogenic compounds include Angiostatin® (U.S. Pat. Nos. 5,639,725; 5,792,845; 5,885,795; 5,733,876; 5,776,704; 5,837,682; 5,861,372, and 5,854,221) and Endostatin (U.S. Pat. No. 5,854,205).

Another compound which has been shown to inhibit angiogenesis is thalidomide. (D'Amato, et al., Proc. Natl. Acad. Sci., 90:4082-85 (1994)). Thalidomide is a hypnosedative that has been successfully used to treat a number of diseases, such as rheumatoid arthritis (Gutierrez-Rodriguez, Arthritis Rheum., 27 (10):1118-21 (1984); Gutierrez-Rodriguez, et al., J. Rheumatol., 16(2):158-63 (1989)), and Behcet's disease (Handley, et al., Br. J. Dermatol., 127 Suppl, 40:67-8 (1992); Gunzler, Med. Hypotheses, 30(2):105-9 (1989)).

Although thalidomide has minimal side effects in adults, it is a potent teratogen. Thus, there are concerns regarding its use in women of child-bearing age. Although minimal, there are a number of side effects that limit the desirability of thalidomide as a treatment. One such side effect is drowsiness. In a number of therapeutic studies, the initial dosage of thalidomide had to be reduced because patients became lethargic and had difficulty functioning normally. Another side effect limiting the use of thalidomide is peripheral neuropathy, in which individuals suffer from numbness and dysfunction in their extremities.

Thus, it is clear that cellular proliferation, particularly endothelial cell proliferation, and most particularly angiogenesis, plays a major role in the metastasis of a cancer. If this abnormal or undesirable proliferation activity could be repressed, inhibited, or eliminated, then the tumor, although present, would not grow. In the disease state, prevention of abnormal or undesirable cellular proliferation and angiogenesis could avert the damage caused by the invasion of the new microvascular system. Therapies directed at control of the cellular proliferative processes could lead to the abrogation or mitigation of these diseases.

What are needed therefore, are methods and compositions that can inhibit abnormal or undesirable cellular proliferation, especially the growth of blood vessels into tumors. Such methods and compositions should be able to overcome the activity of endogenous growth factors in premetastatic tumors and prevent the formation of capillaries in the tumors thereby inhibiting the development of disease and the growth of tumors. Such methods and compositions should also be able to modulate the formation of capillaries in angiogenic processes, such as wound healing and reproduction. Finally, the methods and compositions for inhibiting cellular proliferation should preferably be non-toxic and produce few side effects.

### SUMMARY OF THE INVENTION

Methods and compositions are provided that are effective in inhibiting abnormal or undesirable cell proliferation, particularly endothelial cell proliferation and angiogenesis related to neovascularization and tumor growth. The compositions of the present invention comprise one or more peptide molecules. Certain embodiments of this invention have two or more peptide chains covalently linked to form a dimer; the peptides in the dimer may be identical (homodimer), or they may be different (heterodimer). In certain other embodiments of the present invention, the compositions comprise several peptides (identical or nonidentical) wherein one or more peptides is linked to a chemical moiety capable of modifying its activity. Optionally, the peptides of this invention can be modified with water soluble polymers such as polyethylene glycol (PEG), which can be either branched or linear. More than one water soluble polymer may be attached to the peptides of this invention, and the water soluble polymer(s) may be attached directly to the peptide through covalent modification of one of the side chains, amino terminus, or carboxy terminus of the peptide, or attached to an optional linker, or to a spacer molecules. Certain embodiments of this invention comprise PEG molecules of molecular weight from approximately 5 to 60 kDa, more preferably from approximately 10 to 40 kDa, and even more preferably from 20 to 30 kDa in weight. In embodiments wherein the peptides are modified by additional components, one or more peptides of the composition may be modified; for example, in a homodimer composition, a polymer such as PEG may be attached to just one or both peptides.

Though not wishing to be bound by the following theory, it is believed that by inhibiting endothelial cell proliferation, the compositions described herein are useful for inhibiting tumor growth and metastasis by blocking tumor vascularization.

The methods provided herein for treating diseases and processes mediated by undesired and uncontrolled cell proliferation, such as cancer, involve administering to a human or animal the compositions described herein in a dosage sufficient to inhibit cell proliferation, particularly endothelial cell proliferation. The method is especially useful for treating or repressing the growth of tumors, particularly by inhibiting angiogenesis. Administration of the compositions to a human or animal having prevascularized metastasized tumors is useful for preventing the growth or expansion of such tumors.

Accordingly, it is an object of the present invention to provide methods of treating diseases and processes that are mediated by abnormal or undesirable cellular proliferation.

It is another object of the present invention to provide compositions for treating or repressing the growth of a cancer.

It is yet another object of the present invention to provide a therapy for cancer that has minimal side effects.

It is another object of the present invention to provide methods and compositions for treating diseases and processes that are mediated by angiogenesis.

A further object of the present invention is to provide novel peptides and peptide compositions for treating diseases and processes that are mediated by abnormal or undesirable cellular proliferation.

These and other objects, features and advantages of the present invention will become apparent after a review of the following detailed description of the disclosed embodiments and the appended claims.

### DETAILED DESCRIPTION

The following description includes a preferred presently contemplated mode of carrying out the invention. This description is made for the purpose of illustrating the general principles of the invention and should not be taken in a limiting sense. All publications, references, applications and patents listed or cited herein are incorporated by reference in their entirety.

Compositions and methods for the treatment of diseases and processes that are mediated by, or associated with, abnormal or undesirable cellular proliferation are provided.

The compositions of the present invention generally comprise peptides having antiproliferative activity, including antiangiogenic activity. For delivery to a human or animal, the compositions may further optionally comprise a pharmaceutically acceptable carrier. The term “active peptide” is defined herein as a peptide that inhibits abnormal or undesirable cell proliferation, particularly endothelial cell proliferation as demonstrated and assessed in in vivo or in vitro assays or other known techniques.

In accordance with the methods, the compositions described herein, containing a peptide, optionally combined with a pharmaceutically acceptable carrier, are administered to humans or animals exhibiting undesirable cellular proliferation in an amount sufficient to inhibit the undesirable cell proliferation, particularly endothelial cell proliferation, angiogenesis or an angiogenesis-related disease, such as cancer.

Methods and compositions are provided that are effective in inhibiting abnormal or undesirable cell proliferation, particularly endothelial cell proliferation and angiogenesis, related to neovascularization and tumor growth. The compositions of the present invention comprise peptide molecules. Certain embodiments of the present invention include compositions comprising individual peptides of just one type (monomers), or alternatively two or more peptide chains covalently linked together: certain specific compositions may comprise two identical peptides forming homodimers, two different peptides forming heterodimers, or more than two identical or non-identical peptides forming multimers. Optionally, the peptides of this invention can be modified with water soluble polymers such as polyethylene glycol (PEG) which can be either branched or linear. More than one water soluble polymer may be attached to the peptides of this invention, and the water soluble polymer(s) may be attached directly to the peptide through covalent modification of one of the side chains, amino terminus, or carboxy terminus of the peptide, or attached to an optional linker, or to one or more spacer molecules. Such water soluble polymers may be attached to one or more peptides of the compositions. Certain embodiments of this invention comprise the use of PEG molecules of molecular weight from approximately 5 to 60 kDa, more preferably from approximately 10 to 40 kDa, and even more preferably from approximately 20 to 30 kDa in weight.

Methods and compositions for binding together (i.e. dimerizing or multimerizing) the peptides of the present invention are also provided.

The present invention generally comprises peptides set forth below in Table 1, as SEQ ID NO: 1 through SEQ ID NO: 524. An amino acid with an asterisk denotes the dimerization or multimerization point. The linkers used to dimerize or multimerize the peptides are Lys, Lys-TAT, Ida, GP3, DSS, DST, Ata. The uncommon amino acid 3 letter codes used in Table 1 are: Aib is α-aminoisobutyric acid; Ahx is 6-aminohexanoic acid; Cha is β-cyclohexylalanine; Nal is 1-naphthylalanine. Lower case amino acid letters correspond to the D-amino acid analog of the regular L-amino acid.

TABLE 1

| | | |
|---|---|---|
| | SEQ | IC50 |
| | ID | on |
| Peptide Amino Acid Sequence | NO: | HUVECs |

| | | |
|---|---|---|
| AhxRTRRRRRRQRVRIAYEEIFVRNM | 1 | <10 |
| TKRKRKKQRVKIAYEEIFVKNM | 2 | 10-30 |
| KRKRKKQRVKIAYEEIFVKNM | 3 | 10-30 |
| RKRKKQRVKIAYEEIFVKNM | 4 | 10-30 |
| KTKRKRKKQRVKIAYEEIFVKNM | 5 | <10 |
| KTKRKRKKQRVKIAYEEVFVKNM | 6 | <10 |
| KNKSKGVVKIQRRKAPFVVYESIN | 7 | >30 |
| KKSSKRAKTQRRRKSFVKMYENIH | 8 | >30 |
| KKSSKRAKTQRRRKSFVKMYENIH | 9 | >30 |
| KRKKQRVKIAYEEIFVKNM | 10 | 10-30 |
| RKKQRVKIAYEEIFVKNM | 11 | >30 |
| KKQRVKIAYEEIFVKNM | 12 | <10 |
| KTKRKRKKQRVKIAYEEIFVKN | 13 | <10 |
| KTKRKRKKQRVKIAYEEIFVK | 14 | <10 |
| KTKRKRKKQRVKIAYEEIFV | 15 | <10 |
| KTKRKRKKQRVKIAYEEIF | 16 | 10-30 |
| KTKRKRKKQRVKIAYEEI | 17 | 10-30 |
| KTKRKRKKQRVKIAYEE | 18 | 10-30 |
| TKRKRKKQRVKIAYEEIFVQNM | 19 | >30 |
| KRKRKKQRVKIAYEEIFVQNM | 20 | >30 |
| RKRKKQRVKIAYEEIFVQNM | 21 | >30 |
| KRKKQRVKIAYEEIFVQNM | 22 | >30 |
| RKKQRVKIAYEEIFVQNM | 23 | 10-30 |
| KKQRVKIAYEEIFVQNM | 24 | >30 |
| KTKRKRKKQRVKIAYEEIFVQN | 25 | <10 |
| KTKRKRKKQRVKIAYEEIFVQ | 26 | <10 |
| AcRTRRRRRRQRVRIAYEEIFVRNMAhxK | 27 | 10-30 |
| [AcRTRRRRRRQRVRIAYEEIFVRNMAhXK*]2-IdaBoc | 28 | <10 |
| KTKRKRKKQAYEEIFVQNM | 29 | >30 |
| KTKRKRKKQRYEEIFVQNM | 30 | >30 |
| KTKRKRKKQRVEEIFVQNM | 31 | >30 |
| KTKRKRKKQRVKEIFVQNM | 32 | >30 |
| KTKRKRKKQRVKIIFVQNM | 33 | <10 |
| KTKRKRKKQRVKIAFVQNM | 34 | 10-30 |
| KTKRKRKKQRVKIAYE | 35 | >30 |
| KTKRKRKKQRVKIAY | 36 | >30 |
| KTKRKRKKQRVKIA | 37 | >30 |
| KTKRKRKKQRVKI | 38 | >30 |
| KTKRKRKKQRVKIAYVQNM | 39 | 10-30 |
| KTKRKRKKQRVKIAYEQNM | 40 | >30 |
| KTKRKRKKQRVKIAYEENM | 41 | >30 |
| KTKRKRKKQRVKIAYEEIM | 42 | >30 |
| VKIAYEEIFVQNM | 43 | >30 |
| RVKIAYEEIFVQNM | 44 | >30 |
| KTKRKRKKQRVKIAYEEIFVQAA | 45 | 10-30 |
| KTKRKRKKQRVKIAYEEIFAANM | 46 | 10-30 |
| KTKRKRKKQRVKIAYEEAAVQNM | 47 | >30 |
| KTKRKRKKQRVKIAYAAIFVQNM | 48 | >30 |
| KTKRKRKKQRVKIAAEEIFVQNM | 49 | >30 |
| KTKRKRKKQRVAAAYEEIFVQNM | 50 | >30 |
| [AcKTKRKRKKQRVKIAYEEIFVQNMK*]2-Ida | 51 | <10 |
| KTKRKRVKIAYEEIFVQNM | 52 | >30 |
| KTKRKRKAARVKIAYEEIFVQNM | 53 | >30 |
| KTKRKAAKQRVKIAYEEIFVQNM | 54 | >30 |
| KTKAARKKQRVKIAYEEIFVQNM | 55 | >30 |
| KAARKRKKQRVKIAYEEIFVQNM | 56 | 10-30 |
| KTKKQRVKIAYEEIFVQNM | 57 | >30 |
| KTKRKRKKIAYEEIFVQNM | 58 | >30 |
| [Ahx*RTRRRRRRQRVRIAYEEIFVRNM]2-Ida | 59 | >30 |
| TKRKRKKQRVKIAYEEIFV | 60 | 10-30 |
| KRKRKKQRVKIAYEEIFV | 61 | 10-30 |
| RKRKKQRVKIAYEEIFV | 62 | >30 |
| KRKKQRVKIAYEEIFV | 63 | <10 |
| RKKQRVKIAYEEIFV | 64 | 10-30 |
| TKRKRKKQRVKIAYEEIF | 65 | >30 |
| KRKRKKQRVKIAYEEIF | 66 | 10-30 |
| RKRKKQRVKIAYEEIF | 67 | >30 |
| KRKKQRVKIAYEEIF | 68 | >30 |
| RKKQRVKIAYEEIF | 69 | >30 |
| KKQRVKIAYEEIF | 70 | >30 |
| KKKKKMPKLRFASRIRKIRKKQF | 71 | 10-30 |
| AAKRKRKKQRVKIAYEEIFVQNM | 72 | 10-30 |
| KTKRKRKKQAAKIAYEEIFVQNM | 73 | >30 |
| TKRKRKKQQRVKIAYEEIFV | 74 | >30 |
| KRKRKKQQRVKIAYEEIFV | 75 | 10-30 |
| RKRKKQQRVKIAYEEIFV | 76 | >30 |
| KRKKQQRVKIAYEEIFV | 77 | >30 |
| RKKQQRVKIAYEEIFV | 78 | >30 |
| KTKRKRKKQRVKIIF | 79 | >30 |
| TKRKRKKQRVKIIF | 80 | >30 |
| KRKRKKQRVKIIF | 81 | 10-30 |
| RKRKKQRVKIIF | 82 | >30 |
| KRKKQRVKIIF | 83 | >30 |
| RKKQRVKIIF | 84 | >30 |
| KTKRKRKKQRVKIIFV | 85 | <10 |
| TKRKRKKQRVKIIFV | 86 | 10-30 |
| KRKRKKQRVKIIFV | 87 | <10 |
| KRKKQRVKIIFV | 88 | >30 |
| RKKQRVKIIFV | 89 | >30 |
| RKRKKQRVKIIFV | 90 | 10-30 |
| [KTKRKRKKQRVKIIFV*]2-K | 91 | <10 |
| [TKRKRKKQRVKIIFV*]2-K | 92 | <10 |
| [KRKRKKQRVKIIFV*]2-K | 93 | <10 |
| [RKRKKQRVKIIFV*]2-K | 94 | <10 |
| [KRKKQRVKIIFV*]2-K | 95 | <10 |
| [RKKQRVKIIFV*]2-K | 98 | <10 |
| [KTKRKRKKQRVKIIF*]2-K | 97 | <10 |
| [TKRKRKKQRVKIIF*]2-K | 98 | <10 |
| [KRKRKKQRVKIIF*]2-K | 99 | <10 |
| [RKRKKQRVKIIF*]2-K | 100 | <10 |
| [KRKKQRVKIIF*]2-K | 101 | <10 |
| [RKKQRVKIIF*]2-K | 102 | <10 |
| [KTKRKRKKQRVKIIFVQNM*]2-K | 103 | <10 |
| [AcKTKRKRKKQRVKIIFVQNM*]2-K | 104 | <10 |
| KTKRKRKKQRVKIIFVQNA | 105 | <10 |
| KTKRKRKKQRVKIIFVQAM | 106 | <10 |
| KTKRKRKKQRVKIIFVANM | 107 | <10 |
| KTKRKRKKQRVKIIFAQNM | 108 | 10-30 |
| KTKRKRKKQRVKIIAVQNM | 109 | >30 |
| KTKRKRKKQRVKIAFVQNM | 110 | 10-30 |
| KTKRKRKKQRVKAIFVQNM | 111 | 10-30 |
| KTKRKRKKQRVAIIFVQNM | 112 | <10 |
| KTKRKRKKQRAKIIFVQNM | 113 | <10 |
| KTKRKRKKQAVKIIFVQNM | 114 | >30 |
| KTKRKRKKARVKIIFVQNM | 115 | <10 |
| KTKRKRKAQRVKIIFVQNM | 116 | <10 |
| KTKRKRAKQRVKIIFVQNM | 117 | <10 |
| KTKRKAKKQRVKIIFVQNM | 118 | <10 |
| KTKRARKKQRVKIIFVQNM | 119 | <10 |
| KTKAKRKKQRVKIIFVQNM | 120 | <10 |
| KTARKRKKQRVKIIFVQNM | 121 | <10 |
| KAKRKRKKQRVKIIFVQNM | 122 | <10 |
| ATKRKRKKQRVKIIFVQNM | 123 | <10 |
| [KTKRKRKKQRVKIAYEEIFV*]2-K | 124 | <10 |
| KRKRKKQRVKIAYEEIF*]2-K | 125 | <10 |
| [TKRKRKKQRVKIAYEEIF*]2-K | 126 | <10 |
| [KTKRKRKKQRVKIAYEEIF*]2-K | 127 | <10 |
| [KTKRKRKKQRVKIAhxAhxIF*]2-K | 128 | <10 |
| [TKRKRKKQRVKIAhxAhxIF*]2-K | 129 | <10 |
| [TRRRRRRQRVRIIF*]2-K | 130 | <10 |
| [RRRRRRQRVRIIF*]2-K | 131 | <10 |
| [RRRRRQRVRIIF*]2-K | 132 | <10 |
| [RRRRQRVRIIF*]2-K | 133 | <10 |
| [RRRQRVRIIF*]2-K | 134 | <10 |
| [KRKRKKQRVKIAhXAhxIF*]2-K | 135 | <10 |
| KTKRKRKKQRVKRAYEEIF | 136 | >30 |
| KTKRKRIKQRVKRAYEEIF | 137 | >30 |
| [RKRKKQRVKIIFA*]2-K | 138 | <10 |
| [RKRKKQRVKIIAV*]2-K | 139 | <10 |
| [RKRKKQRVKIAFV*]2-K | 140 | <10 |
| [RKRKKQRVKAIFV*]2-K | 141 | <10 |
| [RKRKKQRVAIIFV*]2-K | 142 | <10 |
| [RKRKKQRAKIIFV*]2-K | 143 | <10 |
| [RKRKKQAVKIIFV*]2-K | 144 | <10 |
| [RKRKKARVKIIFV*]2-K | 145 | <10 |
| [RKRKAQRVKIIFV*]2-K | 146 | <10 |
| [RKRAKQRVKIIFV*]2-K | 147 | <10 |
| [RKAKKQRVKIIFV*]2-K | 148 | <10 |
| [RARKKQRVKIIFV*]2-K | 149 | <10 |
| [AKRKKQRVKIIFV*]2-K | 150 | <10 |
| [NKRKKQRVKIIFV*]2-K | 151 | <10 |
| [QKRKKQRVKIIFV*]2-K | 152 | <10 |
| [GKRKKQRVKIIFV*]2-K | 153 | <10 |
| [HKRKKQRVKIIFV*]2-K | 154 | <10 |
| [IKRKKQRVKIIFV*]2-K | 155 | <10 |
| [LKRKKQRVKIIFV*]2-K | 156 | <10 |
| [FKRKKQRVKIIFV*]2-K | 157 | <10 |
| [SKRKKQRVKIIFV*]2-K | 158 | <10 |
| [TKRKKQRVKIIFV*]2-K | 159 | <10 |
| [YKRKKQRVKIIFV*]2-K | 160 | <10 |
| [VKRKKQRVKIIFV*]2-K | 161 | <10 |
| [KKQRVKIIFV*]2-K | 162 | <10 |
| [KQRVKIIFV*]2-K | 163 | <10 |
| [KKQRVKIIF*]2-K | 164 | <10 |
| [KQRVKIIF*]2-K | 165 | <10 |
| BiotinTKRKRKKQRVKIIFV | 166 | 10-30 |
| [AcRRRRRQRVRIIF*]2-Ida | 167 | <10 |
| [AcRRRRRQRVRIIF*]2-GP3 | 168 | <10 |
| Ida[Ac-K*RRRRRQRVRIIF]2 | 169 | <10 |
| [AcRRRRRQRVRTIF*]2-IdaPEG20kDa | 170 | <10 |
| [AcRRRRRQRVRIIF*]2-GP3(PEG20kDa)2 | 171 | <10 |
| PEG20kDa-Ida[Ac-K*RRRRRQRVRIIF*]2 | 172 | <10 |
| [AcARRRRQRVRIIF*]2-K | 173 | <10 |
| [AcRARRRQRVRIIF*]2-K | 174 | <10 |
| [AcRRARRQRVRIIF*]2-K | 175 | <10 |
| [AcRRRARQRVRIIF*]2-K | 176 | <10 |
| [AcRRRRAQRVRIIF*]2-K | 177 | <10 |
| [AcRRRRRQAVRIIF*]2-K | 178 | <10 |
| [AcRRRRRQRVAIIF*]2-K | 179 | <10 |
| [RQRIRVRIRFRVR*]2-K | 180 | <10 |
| [FRRVRRIVRRIQR*]2-K | 181 | <10 |
| [RIQRRFRRVIRRV*]2-K | 182 | <10 |
| [RFVRIRIRQRRR*]2-K | 183 | <10 |
| [AcHRRRRQRVRIIF*]2-K | 184 | <10 |
| [AcRHRRRQRVRIIF*]2-K | 185 | <10 |
| [AcRRHRRQRVRIIF*]2-K | 186 | <10 |
| [AcRRRHRQRVRIIF*]2-K | 187 | <10 |
| [AcRRRRHQRVRIIF*]2-K | 188 | <10 |
| [AcRRRRRQHVRIIF*]2-K | 189 | <10 |
| [AcRRRRRQRVHIIF*]2-K | 190 | <10 |
| [AcAARRRQRVRIIF*]2-K | 191 | <10 |
| [AcARARRQRVRIIF*]2-K | 192 | <10 |
| [AcARRARQRVRIIF*]2-K | 193 | <10 |
| [AcARRRAQRVRIIF*]2-K | 194 | <10 |
| [AcARRRRQAVRIIF*]2-K | 195 | <10 |
| [AcARRRRQRVAIIF*]2-K | 196 | <10 |
| [AcRAARRQRVRIIF*]2-K | 197 | <10 |
| [AcRARARQRVRIIF*]2-K | 198 | <10 |
| [AcRARRAQRVRIIF*]2-K | 199 | <10 |
| [AcRARRRQAVRIIF*]2-K | 200 | <10 |
| [AcRARRRQRVAIIF*]2-K | 201 | <10 |
| [AcRRAARQRVRIIF*]2-K | 202 | <10 |
| [AcRRARAQRVRIIF*]2-K | 203 | <10 |
| [AcRRARRQAVRIIF*]2-K | 204 | <10 |
| [AcRRARRQRVAIIF*]2-K | 205 | <10 |
| [AcRRRAAQRVRIIF*]2-K | 206 | <10 |
| [AcRRRARQAVRIIF*]2-K | 207 | <10 |
| [AcRRRARQRVAIIF*]2-K | 208 | <10 |
| [AcRRRRAQAVRIIF*]2-K | 209 | <10 |
| [AcRRRRAQRVAIIF*]2-K | 210 | <10 |
| [AcRRRRRQAVAIIF*]2-K | 211 | <10 |
| [AcAARRRQRVAIIF*]2-K | 212 | >30 |
| [AcARARRQRVAIIF*]2-K | 213 | <10 |
| [AcARRARQRVAIIF*]2-K | 214 | <10 |
| [AcARRRAQRVAIIF*]2-K | 215 | <10 |
| [AcARRRRQAVAIIF*]2-K | 216 | 10-30 |
| [AcAHHHHQHVAIIF*]2-K | 217 | >30 |
| [AcRARRRQAVAIIF*]2-K | 218 | <10 |
| [AcRRARRQAVAIIF*]2-K | 219 | <10 |
| [AcRRRARQAVAIIF*]2-K | 220 | <10 |
| [AcRRRRAQAVAIIF*]2-K | 221 | <10 |
| [AcHHHHHQAVAIIF*]2-K | 222 | >30 |
| [AcARRRRQRVAIGFK*]2-Ida | 223 | >30 |
| [AcARRRRQRVAIHFK*]2-Ida | 224 | <10 |
| [AcARRRRQRVAINFK*]2-Ida | 225 | <10 |
| [AcARRRRQRVAITFK*]2-Ida | 226 | <10 |
| [AcARRRRQRVAIWFK*]2-Ida | 227 | <10 |
| [AcARRRRQRVAIYFK*]2-Ida | 228 | <10 |
| [AcHHRRRQRVRIIF*]2-K | 229 | <10 |
| [AcHRHRRQRVRIIF*]2-K | 230 | <10 |
| [AcHRRHRQRVRIIF*]2-K | 231 | <10 |
| [AcHRRRHQRVRIIF*]2-K | 232 | <10 |
| [AcHRRRRQHVRIIF*]2-K | 233 | <10 |
| [AcHRRRRQRVHIIF*]2-K | 234 | <10 |
| [AcRHHRRQRVRIIF*]2-K | 235 | <10 |
| [AcRHRHRQRVRIIF*]2-K | 236 | <10 |
| [AcRHRRHQRVRIIF*]2-K | 237 | <10 |
| [AcRHRRRQHVRIIF*]2-K | 238 | <10 |
| [AcRHRRRQRVHIIF*]2-K | 239 | <10 |
| [AcRRHHRQRVRIIF*]2-K | 240 | <10 |
| [AcRRHRHQRVRIIF*]2-K | 241 | <10 |
| [AcRRHRRQHVRIIF*]2-K | 242 | <10 |
| [AcRRHRRQRVHIIF*]2-K | 243 | <10 |
| [AcRRRHRQHVRIIF*]2-K | 244 | <10 |
| [AcRRRHRQRVHIIF*]2-K | 245 | 10-30 |
| [AcRRRRHQHVRIIF*]2-K | 246 | <10 |
| [AcRRRRHQRVHIIF*]2-K | 247 | <10 |
| [AcRRRRRQHVHIIF*]2-K | 248 | <10 |
| [AcRRRRRQAVAIEFK*]2-Ida | 249 | 10-30 |
| [AcRRRRRQAVAIHFK*]2-Ida | 250 | <10 |
| [AcRRRRRQAVAIPFK*]2-Ida | 251 | >30 |
| [AcARRRRQRVAIIFk*]2-Ida | 252 | <10 |
| [AcARRRRQRVAIIfK*]2-Ida | 253 | <10 |
| [AcARRRRQRVAIiFK*]2-Ida | 254 | <10 |
| [AcARRRRQRVAiIFK*]2-Ida | 255 | <10 |
| [AcARRRRQRVaIIFK*]2-Ida | 256 | <10 |
| [AcARRRRQRvAIIFK*]2-Ida | 257 | <10 |
| [AcARRRRQrVAIIFK*]2-Ida | 258 | <10 |
| [AcARRRRqRVAIIFK*]2-Ida | 259 | <10 |
| [AcARRRrQRVAIIFK*]2-Ida | 260 | <10 |
| [AcARRrRQRVAIIFK*]2-Ida | 261 | <10 |
| [AcARrRRQRVAIIFK*]2-Ida | 262 | <10 |
| [AcArRRRQRVAIIFK*]2-Ida | 263 | <10 |
| [AcaRRRRQRVAIIFK*]2-Ida | 264 | <10 |
| [AcRRRRRQAVAIGFK*]2-Ida | 265 | <10 |
| [AcRRRRRQAVAIWFK*]2-Ida | 266 | <10 |
| [AcRRRRRQAVAIYFK*]2-Ida | 267 | <10 |
| [AcARRRRQRVAIPFK*]2-Ida | 268 | >30 |
| [AcARRRRQRVAIEFK*]2-Ida | 269 | 10-30 |
| [AcRRRRRQAVAINFK*]2-Ida | 270 | <10 |
| [AcARRRRQRVAIYF*]2-K | 271 | <10 |
| [AcRARRRQAVAINFK*]2-Ida | 272 | <10 |
| [AcRARRRQAVAITFK*]2-Ida | 273 | <10 |
| [AcRARRRQAVAIWFK*]2-Ida | 274 | <10 |
| [AcRARRRQAVAIYFK*]2-Ida | 275 | <10 |
| [AcHRRRRQHVRIYFK*]2-Ida | 276 | <10 |
| [AcHRRRRQRVHIYFK*]2-Ida | 277 | <10 |
| [AcRRRRRQHVHIYFK*]2-Ida | 278 | <10 |
| [AcRRRRRQRVRIYFK*]2-Ida | 279 | <10 |
| [AcRRRRAQAVAIYFK*]2-Ida | 280 | <10 |
| [AcRRRRHQAVAIYFK*]2-Ida | 281 | <10 |
| [AcRRRRAQHVAIYFK*]2-Ida | 282 | <10 |
| [AcRRRRAQAVHIYFK*]2-Ida | 283 | <10 |
| [AcRRRRHQHVAIYFK*]2-Ida | 284 | <10 |
| [AcRRRRHQAVHIYFK*]2-Ida | 285 | <10 |
| [AcRRRRAQHVHIYFK*]2-Ida | 286 | <10 |
| [AcRRRRHQHVHIYFK*]2-Ida | 287 | <10 |
| [AcARRRRQRVAIyFK*]2-Ida | 288 | >30 |
| [AcARRRRQRVAIlNalFK*]2-Ida | 289 | <10 |
| [AcARRRRQRVAIPhenylGlyFK*]2-Ida | 290 | <10 |
| [AcARRRRQRVAIChaFK*]2-Ida | 291 | <10 |
| [AcARRRRQRVAIHomoPheFK*]2-Ida | 292 | <10 |
| [AcARRRRQRVAIhomopheFK*]2-Ida | 293 | <10 |
| [AcARRRRQRVAI3,4-DichloroPheFK*]2-Ida | 294 | <10 |
| [AcARRRRQRVAI3,3-DiphenylPheFK*]2-Ida | 295 | 10-30 |
| [AcARRRRQRVAI4-ChloroPheFK*]2-Ida | 296 | <10 |
| [AcARRRRQRVAI4-NitroPheFK*]2-Ida | 297 | <10 |
| [AcARRRRQRVAAibFK*]2-Ida | 298 | >30 |
| [AcRRRRRQAVRIGIGFK*]2-Ida | 299 | <10 |
| [AcRRRRRQAVRIPIPFK*]2-Ida | 300 | 10-30 |
| [AcRRRRRQAVRITITFK*]2-Ida | 301 | <10 |
| [AcRRRRRQAVRIAIAFK*]2-Ida | 302 | <10 |
| [AcRRRRRQAVRIQIQFK*]2-Ida | 303 | <10 |
| [AcRRRRRQAVRIDIDFK*]2-Ida | 304 | <10 |
| [AcRRRRRQAVRIIKVN*]2-Ida | 305 | <10 |
| [AcRRRRRQAVRIKFV*]2-Ida | 306 | <10 |
| [AcRRRRRQAVRKIFV*]2-Ida | 307 | <10 |
| [AcRRRRRQAVKIIFV*]2-Ida | 308 | <10 |
| [AcRRRRRQAKRIIFV*]2-Ida | 309 | <10 |
| [AcRRRRRQKVRIIFV*]2-Ida | 310 | <10 |
| AcRRRRRQAVRITITFK | 311 | <10 |
| [AcRARRRQAVAIEFK*]2-Ida | 312 | >30 |
| [AcRARRRQAVAIGFK*]2-Ida | 313 | 10-30 |
| [AcRARRRQAVAIPFK*]2-Ida | 314 | >30 |
| [AcARRRRQAVAIYFK*]2-Ida | 315 | <10 |
| [AcARRRRQAVHIYFK*]2-Ida | 316 | <10 |
| [AcARRRRQHVAIYFK*]2-Ida | 317 | <10 |
| [AcHRRRRQAVAIYFK*]2-Ida | 318 | <10 |
| [AcARRRRQHVHIYFK*]2-Ida | 319 | <10 |
| [AcHRRRRQAVHIYFK*]2-Ida | 320 | <10 |
| [AcHRRRRQHVAIYFK*]2-Ida | 321 | <10 |
| [AcHRRRRQHVHIYFK*]2-Ida | 322 | <10 |
| [AcRHRRRQHVRIYFK*]2-Ida | 323 | <10 |
| [AcRRHRRQHVRIYFK*]2-Ida | 324 | <10 |
| [AcRRRHRQHVRIYFK*]2-Ida | 325 | <10 |
| [AcRRRRHQHVRIYFK*]2-Ida | 326 | <10 |
| [AcHHRRRQHVRIYFK*]2-Ida | 327 | <10 |
| [AcHRHRRQHVRIYFK*]2-Ida | 328 | <10 |
| [AcARRRRQRVAIYFK*]2-GP3 | 329 | <10 |
| [AcHRRHRQHVRIYFK*]2-Ida | 330 | <10 |
| [AcARRRRQRVAIYFK*]2-GP3 | 331 | <10 |
| [AcHRRRHQHVRIYFK*]2-Ida | 332 | <10 |
| [AcRHHRRQHVRIYFK*]2-Ida | 333 | <10 |
| [AcARRRRQRVAIYFK*]2-Ida-(AcOH salt) | 334 | <10 |
| [AcRHRRHQHVRIYFK*]2-Ida | 335 | <10 |
| [AcARRRRQRVAIYFK*]2-Ida-PEG 20 kDa | 336 | <10 |
| [AcRHRHRQHVRIYFK*]2-Ida | 337 | <10 |
| [AcRHRRRQHVHIYFK*]2-Ida | 338 | <10 |
| [AcRRHHRQHVRIYFK*]2-Ida | 339 | <10 |
| [AcRRHRHQHVRIYFK*]2-Ida | 340 | <10 |
| [AcRRHRHQRVHIYFK*]2-Ida | 341 | <10 |
| [AcARRRRQRVAIYd,1-HomoPheK*]2-Ida | 342 | 10-30 |
| [AcARRRRQRVAIYPhenylGlyK*]2-Ida | 343 | <10 |
| [AcARRRRQRVAIYChaK*]2-Ida | 344 | <10 |
| [AcARRRRQRVAIYWK*]2-Ida | 345 | <10 |
| [AcARRRRQRVAIYEK*]2-Ida | 346 | >30 |
| [AcARRRRQRVAIYTK*]2-Ida | 347 | >30 |
| [AcARRRRQRVAIYHK*]2-Ida | 348 | >30 |
| [AcARRRRQRVAIYPK*]2-Ida | 349 | >30 |
| [AcARRRRQRVAIY3,3-diphenylAlaK*]2-Ida | 350 | <10 |
| [AcARRRRQRVAIYGK*]2-Ida | 351 | >30 |
| [AcARRRRQRVAIYIK*]2-Ida | 352 | <10 |
| [AcARRRRQRVAIYYK*]2-Ida | 353 | >30 |
| AcARRRRQRVAIiFK | 354 | >30 |
| AcARRRRqRVAIIFK | 355 | >30 |
| AcArRRRQRVAIIFK | 356 | >30 |
| AcRRRRRQAVRIQIQFK | 357 | 10-30 |
| AcRRRRRQAVRIDIDFK | 358 | >30 |
| [AcRRRHHQHVRIYFK*]2-Ida | 359 | <10 |
| [AcRRRHRQHVHIYFK*]2-Ida | 360 | <10 |
| AcARRRRQRVAIYFK | 362 | >30 |
| [AcARRRRQRVAChaYFK*]2-Ida | 362 | <10 |
| [AcARRRRQRVAWYFK*]2-Ida | 363 | <10 |
| [AcARRRRQRVAEYFK*]2-Ida | 364 | >30 |
| [AcARRRRQRVATYFK*]2-Ida | 365 | >30 |
| [AcARRRRQRVAHYFK*]2-Ida | 366 | >30 |
| [AcARRRRQRVAPYFK*]2-Ida | 367 | >30 |
| [AcARRRRQRVA3,3-diphenylAlaYFK*]2-Ida | 368 | <10 |
| [AcARRRRQRVAQYFK*]2-Ida | 369 | >30 |
| [AcARRRRQRVAFYFK*]2-Ida | 370 | <10 |
| [AcARRRRQRVAYYFK*]2-Ida | 371 | <10 |
| [AcARRRRQRVAHomoPheYFK*]2-Ida | 372 | <10 |
| [AcARRRRQRVAhomopheYFK*]2-Ida | 373 | 10-30 |
| [AcARRRRQRVAPhenylGlyYFK*]2-Ida | 374 | <10 |
| [AcRRRRRQRVRIIFK*]2-Ida-PEG 30 kDa | 375 | <10 |
| [AcARRRRQRVAIYFK*]2-Ida-PEG 30 kDa | 376 | <10 |
| [AcARRRRQRVAIMFK*]2-Ida | 377 | <10 |
| [AcARRRRQRVAIQFK*]2-Ida | 378 | <10 |
| [AcARRRRQRVAISFK*]2-Ida | 379 | <10 |
| [AcARRRRQEVAIYFK*]2-Ida | 380 | <10 |
| [AcARRRRQAVAIYFK*]2-Ida-PEG 30 kDa | 381 | <10 |
| [AcARRRRQRVAIVFK*]2-Ida | 382 | <10 |
| [AcARRRRQFVAIYFK*]2-Ida | 383 | <10 |
| [AcARRRRQGVAIYFK*]2-Ida | 384 | <10 |
| [AcARRRRQMVAIYFK*]2-Ida | 385 | <10 |
| [AcARRRRQNVAIYFK*]2-Ida | 386 | <10 |
| [AcARRRRQAVEIYFK*]2-Ida | 387 | <10 |
| [AcARRRRQAVFIYFK*]2-Ida | 388 | <10 |
| [AcARRRRQAVGIYFK*]2-Ida | 389 | <10 |
| [AcARRRRQAVMIYFK*]2-Ida | 390 | <10 |
| [AcARRRRQAVNIYFK*]2-Ida | 391 | <10 |
| [AcARRRRQAVPIYFK*]2-Ida | 392 | >30 |
| [AcARRRRQAVQIYFK*]2-Ida | 393 | <10 |
| [AcARRRRQAVSIYFK*]2-Ida | 394 | <10 |
| [AcARRRRQAVTIYFK]2-Ida | 395 | <10 |
| [AcARRRRQAVWIYFK*]2-Ida | 396 | <10 |
| [AcARRRRQAVYIYFK*]2-Ida | 397 | <10 |
| [AcARRRRQAVLIYFK*]2-Ida | 398 | <10 |
| [AcARRRRQAVRIYFK*]3-Ata | 399 | <10 |
| [AcDRRRRQAVAIYFK*]2-Ida | 400 | <10 |
| [AcERRRRQAVAIYFK*]2-Ida | 402 | <10 |
| [AcFRRRRQAVAIYFK*]2-Ida | 402 | <10 |
| [AcGRRRRQAVAIYFK*]2-Ida | 403 | <10 |
| [AcIRRRRQAVAIYFK*]2-Ida | 404 | <10 |
| [AcLRRRRQAVAIYFK*]2-Ida | 405 | <10 |
| [AcMRRRRQAVAIYFK*]2-Ida | 406 | <10 |
| [AcNRRRRQAVAIYFK*]2-Ida | 407 | <10 |
| [AcPRRRRQAVAIYFK*]2-Ida | 408 | <10 |
| [AcQRRRRQAVAIYFK*]2-Ida | 409 | <10 |
| [AcSRRRRQAVAIYFK*]2-Ida | 410 | <10 |
| [AcTRRRRQAVAIYFK*]2-Ida | 411 | <10 |
| [AcVRRRRQAVAIYFK*]2-Ida | 412 | <10 |
| [AcWRRRRQAVAIYFK*]2-Ida | 413 | <10 |
| [AcYRRRRQAVAIYFK*]2-Ida | 414 | <10 |
| [AcARRRRQAVRIKFV*]2-Ida | 415 | <10 |
| [AcARRRRQAVRIK*F12-Ida | 416 | <10 |
| [AcRRRRRQAVAIK*FV]2-Ida | 417 | <10 |
| [AcARRRRQPVAIYFK*]2-Ida | 418 | <10 |
| [AcARRRRQQVAIYFK*]2-Ida | 419 | <10 |
| [AcARRRRQSVAIYFK*]2-Ida | 420 | <10 |
| [AcRRRRRQAVAIK*F]2-Ida | 422 | <10 |
| [AcARRRRQTVAIYFK*]2-Ida | 422 | <10 |
| [AcARRRRQRVAIK*FV]2-Ida | 423 | <10 |
| [AcARRRRQWVAIYFK*]2-Ida | 424 | <10 |
| [AcARRRRQRVAIK*F]2-Ida | 425 | <10 |
| [AcARRRRQYVAIYFK*]2-Ida | 426 | <10 |
| [AcARRRRQAVAIK*F]2-Ida | 427 | <10 |
| [AcARRRRQAVAIK*F]2-GP3 | 428 | <10 |
| [AcARRRRQAVAIK*FI]2-Ida | 429 | <10 |
| [AcARRRRQAVAIK*YF]2-Ida | 430 | <10 |
| [AcARRRRQAVAIYK*F]2-Ida | 431 | <10 |
| [(AcRRRRRQAVAIYFK*)2-Ida]2-Ida | 432 | <10 |
| [AcRRRREQAVAIYFK*]2-Ida | 433 | 10-30 |
| [AcRRRRFQAVAIYFK*]2-Ida | 434 | <10 |
| [AcRRRRGQAVAIYFK*]2-Ida | 435 | <10 |
| [AcRRRRLQAVAIYFK*]2-Ida | 436 | <10 |
| [AcRRRRMQAVAIYFK*]2-Ida | 437 | <10 |
| [AcRRRRNQAVAIYFK*]2-Ida | 438 | <10 |
| [AcRRRRPQAVAIYFK*]2-Ida | 439 | <10 |
| [AcRRRRSQAVAIYFK*]2-Ida | 440 | <10 |
| [AcRRRRTQAVAIYFK*]2-Ida | 441 | <10 |
| [AcRRRRVQAVAIYFK*]2-Ida | 442 | <10 |
| [AcRRRRWQAVAIYFK*]2-Ida | 443 | <10 |
| [AcRRRRYQAVAIYFK*]2-Ida | 444 | <10 |
| YGRKKRRQRRR | 445 | >30 |
| [AcRRRRRQAVAIYFK*]3-Ata | 446 | <10 |
| [AcARRRRQAVRIiFK*]3-Ata | 447 | <10 |
| [AcRRRRRQAAAAAAK*]2-Ida | 448 | >30 |
| [AcRRRRRAAAAAAAK*]2-Ida | 449 | >30 |
| [AcARRRRQAVAILFK*]2-Ida | 450 | <10 |
| [AcNRRRRQAVAIVFK*]2-Ida | 451 | >30 |
| [AcPRRRRQAVAIVFK*]2-Ida | 452 | <10 |
| [AcQRRRRQAVAIVFK*]2-Ida | 453 | <10 |
| [AcSRRRRQAVAIVFK*]2-Ida | 454 | <10 |
| [AcTRRRRQAVAIVFK*]2-Ida | 455 | <10 |
| [AcK*RRRRQAVAIVF]2-Ida | 456 | >30 |
| [AcPRRRRQAVAIYFK*]3-Ata | 457 | >30 |
| [AcK*ARRRRQAVAIYF]2-Ida | 458 | >30 |
| [AcK*RRRRQAVAIYF]2-Ida | 459 | >30 |
| [AcARRK*RQAVAIYF]2-Ida | 460 | 10-30 |
| [AcARRRK*QAVAIYF]2-Ida | 462 | 10-30 |
| [AcARRRRK*AVAIYF]2-Ida | 462 | 10-30 |
| [AcARRARQAVAIYFK*]2-Ida | 463 | <10 |
| [AcARRERQAVAIYFK*]2-Ida | 464 | <10 |
| [AcARRQRQAVAIYFK*]2-Ida | 465 | <10 |
| [AcAK*RRRQAVAIYF]2-Ida | 466 | >30 |
| [AcARK*RRQAVAIYF]2-Ida | 467 | >30 |
| [AcARRIRQAVAIYFK*]2-Ida | 468 | >30 |
| AcARRRRQRVAIYFKARRRRQRVAIYFK | 469 | <10 |
| [AcPRRRDQAVAIYFK*]2-Ida | 470 | >30 |
| [AcPRRRFQAVAIYFK*]2-Ida | 471 | >30 |
| [AcPRRRGQAVAIYFK*]2-Ida | 472 | >30 |
| [AcPRRRLQAVAIYFK*]2-Ida | 473 | >30 |
| [AcPRRRMQAVAIYFK*]2-Ida | 474 | 10-30 |
| [AcPRRRNQAVAIYFK*]2-Ida | 475 | <10 |
| [AcPRRRPQAVAIYFK*]2-Ida | 476 | <10 |
| [AcPRRRSQAVAIYFK*]2-Ida | 477 | 10-30 |
| [AcPRRRTQAVAIYFK*]2-Ida | 478 | 10-30 |
| [AcPRRRVQAVAIYFK*]2-Ida | 479 | 10-30 |
| [AcPRRRWQAVAIYFK*]2-Ida | 480 | 10-30 |
| [AcPRRRYQAVAIYFK*]2-Ida | 481 | 10-30 |
| [AcPRRRRQAVAIK*F]2-Ida | 482 | <10 |
| [AcQRRRRQAVAIK*F]2-Ida | 483 | <10 |
| [AcARRRNQAVAIK*F]2-Ida | 484 | <10 |
| [AcARRRPQAVAIK*F]2-Ida | 485 | <10 |
| [AcARRRRQSVAIK*F]2-Ida | 486 | <10 |
| [AcARRRRQPVAIK*F]2-Ida | 487 | 10-30 |
| [AcPRRRNQAVAIK*F]2-Ida | 488 | 10-30 |
| [AcPRRRPQAVAIK*F]2-Ida | 489 | <10 |
| [AcPRRRRQSVAIK*F]2-Ida | 490 | 10-30 |
| [AcPRRRRQPVAIK*F]2-Ida | 491 | 10-30 |
| [AcARRRNQSVAIK*F]2-Ida | 492 | 10-30 |
| [AcARRRNQPVAIK*F]2-Ida | 493 | >30 |
| [AcPRNRRQAVAIYFK*]2-Ida | 494 | 10-30 |
| [AcPRERRQAVAIYFK*]2-Ida | 495 | >30 |
| [AcPRHRRQAVAIYFK*]2-Ida | 496 | 10-30 |
| [AcPRLRRQAVAIYFK*]2-Ida | 497 | 10-30 |
| [AcPRSRRQAVAIYFK*]2-Ida | 498 | <10 |
| [AcPRYRRQAVAIYFK*]2-Ida | 499 | <10 |
| [AcPRRNRQAVAIYFK*]2-Ida | 500 | <10 |
| [AcPRRERQAVAIYFK*]2-Ida | 501 | <10 |
| [AcPRRHRQAVAIYFK*]2-Ida | 502 | 10-30 |
| [AcPRRLRQAVAIYFK*]2-Ida | 503 | <10 |
| [AcPRRSRQAVAIYFK*]2-Ida | 504 | 10-30 |
| [AcPRRYRQAVAIYFK*]2-Ida | 505 | <10 |
| [AcARRRRQRVAIYFK*GGRGDSP]2-Ida | 506 | <10 |
| [AcRGDSPGGARRRRQRVAIYFK*]2-Ida | 507 | <10 |
| [AcCNGRCGGARRRRQRVAIYFK*]2-Ida | 508 | <10 |
| [AcARRRRARVAIYFK*]2-Ida | 509 | <10 |
| [AcARRRRERVAIYFK*]2-Ida | 510 | <10 |
| [AcARRRRFRVAIYFK*]2-Ida | 511 | 10-30 |
| [AcARRRRGRVAIYFK*]2-Ida | 512 | <10 |
| [AcARRRRIRVAIYFK*]2-Ida | 513 | 10-30 |
| [AcARRRRMRVAIYFK*]2-Ida | 514 | <10 |
| [AcARRRRNRVAIYFK*]2-Ida | 515 | <10 |
| [AcARRRRPRVAIYFK*]2-Ida | 516 | <10 |
| [AcARRRRTRVAIYFK*]2-Ida | 517 | 10-30 |
| [AcARRRRVRVAIYFK*]2-Ida | 518 | >30 |
| [AcARRRRWRVAIYFK*]2-Ida | 519 | 10-30 |
| [AcARRRRYRVAIYFK*]2-Ida | 520 | <10 |
| [AcARRRRQRVAIYFK*]2-DSS | 521 | <10 |
| [AcARRRRQRVAIYFK*]2-DST | 522 | <10 |
| [AcARRRRQRVAIYFK*]2-Ida-[AcKGGRGDSP] | 523 | <10 |
| [AcARRRRQRVAIYFK*]2-Ida-PEG 3.4 kDa- | 524 | <10 |
| [AcKGGRGDSP] | | |

The peptide monomers of the invention may be oligomerized using the biotin/streptavidin system. Biotinylated analogs of peptide monomers may be synthesized by standard techniques known to those skilled in the art. For example, the peptide monomers may be C-terminally biotinylated. These biotinylated monomers are then oligomerized by incubation with streptavidin [e.g., at a 4:1 molar ratio at room temperature in phosphate buffered saline (PBS) or HEPES-buffered RPMI medium (Invitrogen) for 1 hour]. In a variation of this process, biotinylated peptide monomers may be oligomerized by incubation with any one of a number of commercially available anti-biotin antibodies [e.g., goat anti-biotin IgG from Kirkegaard & Perry Laboratories, Inc. (Washington, D.C.)].

In certain embodiments, the peptide monomers of the invention are dimerized or multimerized by covalent attachment to at least one linker moiety. The linker moiety is preferably, although not necessarily, a C1-12 linking moiety optionally terminated with one or two —NH— linkages and optionally substituted at one or more available carbon atoms with a lower alkyl substituent. Preferably the linker comprises —NH—R—NH— wherein R is a lower (C1-6) alkylene substituted with a functional group, such as a carboxyl group or an amino group, that enables binding to another molecular moiety (e.g., as may be present on the surface of a solid support during peptide synthesis or to a PK-modifying agent such as PEG). In certain embodiments the linker is a lysine residue. In certain other embodiments, the linker bridges the C-termini of two peptide monomers, by simultaneous attachment to the C-terminal amino acid of each monomer. In other embodiments, the linker bridges the peptides by attaching to the side chains of amino acids not at the C-termini. When the linker attaches to a side chain of an amino acid not at the C-termini of the peptides, the side chain preferably contains an amine, such as those found in lysine, and the linker contains two or more carboxy groups capable of forming an amide bond with the peptides.

The terms “alkyl”, “alkoxy”, “hydroxyalkyl”, “alkoxyalkyl”, and “alkoxycarbonyl”, used alone or as part of a larger moiety includes both straight and branched chains containing one to twelve carbon atoms. The terms “alkenyl” and “alkynyl” used alone or as part of a larger moiety shall include both straight and branched chains containing two to twelve carbon atoms. The term “cycloalkyl” used alone or as part of a larger moiety shall include cyclic C3-C12 hydrocarbons which are completely saturated or which contain one or more units of unsaturation, but which are not aromatic. Lower alkyl refers to an alkyl group containing 1-6 carbons.

The term “amino” refers to an NH2 group.

The term “alkylamino” or “aminoalkyl” refers to an amino group wherein one of the hydrogen atoms is replaced by an alkyl group.

The term “dialkylamino” or “aminodialkyl” refers to an amino group wherein the hydrogen atoms are replaced by alkyl groups,

wherein the alkyl group may be the same or different.

The term “halogen” means F, Cl, Br, or I.

The term “heteroatom” means nitrogen, oxygen, or sulfur and includes any oxidized form of nitrogen and sulfur, and the quaternized form of any basic nitrogen. Also the term “nitrogen” includes a substitutable nitrogen of a heterocyclic ring. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl) or NR+ (as in N-substituted pyrrolidinyl).

The terms “carbocycle”, “carbocyclyl”, “carbocyclo”, or “carbocyclic” as used herein means an aliphatic ring system having three to fourteen members. The terms “carbocycle”, “carbocyclyl”, “carbocyclo”, or “carbocyclic” whether saturated or partially unsaturated, also refers to rings that are optionally substituted. The terms “carbocycle”, “carbocyclyl”, “carbocyclo”, or “carbocyclic” also include aliphatic rings that are fused to one or more aromatic or nonaromatic rings, such as in a decahydronaphthyl or tetrahydronaphthyl, where the radical or point of attachment is on the aliphatic ring.

The term “aryl” used alone or as part of a larger moiety as in “aralkyl”, “aralkoxy”, or “aryloxyalkyl”, refers to aromatic ring groups having six to fourteen members, such as phenyl, benzyl, phenethyl, 1-naphthyl, 2-naphthyl, 1-anthracyl and 2-anthracyl. The term “aryl” also refers to rings that are optionally substituted. The term “aryl” may be used interchangeably with the term “aryl ring”. “Aryl” also includes fused polycyclic aromatic ring systems in which an aromatic ring is fused to one or more rings. Examples include 1-naphthyl, 2-naphthyl, 1-anthracyl and 2-anthracyl. Also included within the scope of the term “aryl”, as it is used herein, is a group in which an aromatic ring is fused to one or more non-aromatic rings, such as in an indanyl, phenanthridinyl, or tetrahydronaphthyl, where the radical or point of attachment is on the aromatic ring.

The term “heterocycle”, “heterocyclyl”, or “heterocyclic” as used herein includes non-aromatic ring systems having four to fourteen members, preferably five to ten, in which one or more ring carbons, preferably one to four, are each replaced by a heteroatom. Examples of heterocyclic rings include 3-1H-benzimidazol-2-one, (1-substituted)-2-oxo-benzimidazol-3-yl, 2-tetrahydro-furanyl, 3-tetrahydrofuranyl, 2-tetrahydropyranyl, 3-tetrahydropyranyl, 4-tetra-hydropyranyl, [1,3]-dioxalanyl, [1,3]-dithiolanyl, [1,3]-dioxanyl, 2-tetra-hydro-thiophenyl, 3-tetrahydrothiophenyl, 2-morpholinyl, 3-morpholinyl, 4-morpholinyl, 2-thiomorpholinyl, 3-thiomorpholinyl, 4-thiomorpholinyl, 1-pyrrolidinyl, 2-pyrrolidinyl, 3-pyrrolidinyl, 1-piperazinyl, 2-piperazinyl, 1-piperidinyl, 2-piperidinyl, 3-piperidinyl, 4-piperidinyl, 4-thiazolidinyl, diazolonyl, N-substituted diazolonyl, 1-phthalimidinyl, benzoxanyl, benzopyrrolidinyl, benzopiperidinyl, benzoxolanyl, benzothiolanyl, and benzothianyl. Also included within the scope of the term “heterocyclyl” or “heterocyclic”, as it is used herein, is a group in which a non-aromatic heteroatom-containing ring is fused to one or more aromatic or non-aromatic rings, such as in an indolinyl, chromanyl, phenanthridinyl, or tetrahydroquinolinyl, where the radical or point of attachment is on the non-aromatic heteroatom-containing ring. The term “heterocycle”, “heterocyclyl”, or “heterocyclic” whether saturated or partially unsaturated, also refers to rings that are optionally substituted.

The term “heteroaryl”, used alone or as part of a larger moiety as in “heteroaralkyl” or “heteroarylalkoxy”, refers to heteroaromatic ring groups having five to fourteen members. Examples of heteroaryl rings include 2-furanyl, 3-furanyl, 3-furazanyl, N-imidazolyl, 2-imidazolyl, 4-imidazolyl, 5-imidazolyl, 3-isoxazolyl, 4-isoxazolyl, 5-isoxazolyl, 2-oxadiazolyl, 5-oxadiazolyl, 2-oxazolyl, 4-oxazolyl, 5-oxazolyl, 1-pyrrolyl, 2-pyrrolyl, 3-pyrrolyl, 1-pyrazolyl, 2-pyrazolyl, 3-pyrazolyl, 2-pyridyl, 3-pyridyl, 4-pyridyl, 2-pyrimidyl, 4-pyrimidyl, 5-pyrimidyl, 3-pyridazinyl, 2-thiazolyl, 4-thiazolyl, 5-thiazolyl, 5-tetrazolyl, 2-triazolyl, 5-triazolyl, 2-thienyl, 3-thienyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, indazolyl, isoindolyl, acridinyl, and benzoisoxazolyl. Also included within the scope of the term “heteroaryl”, as it is used herein, is a group in which a heteroatomic ring is fused to one or more aromatic or nonaromatic rings where the radical or point of attachment is on the heteroaromatic ring. Examples include tetrahydroquinolinyl, tetrahydroisoquino-linyl, and pyrido[3,4-d]pyrimidinyl. The term “heteroaryl” also refers to rings that are optionally substituted. The term “heteroaryl” may be used interchangeably with the term “heteroaryl ring” or the term “heteroaromatic”.

An aryl (including aralkyl, aralkoxy, aryloxyalkyl and the like) or heteroaryl (including heteroaralkyl and heteroarylalkoxy and the like) group may contain one or more substituents. Examples of suitable substituents on any unsaturated carbon atom of an aryl, heteroaryl, aralkyl, or heteroaralkyl group include a halogen, —R0, —OR0, —SR0, 1,2-methylene-dioxy, 1,2-ethylenedioxy, protected OH (such as acyloxy), phenyl (Ph), substituted Ph, —O(Ph), substituted —O(Ph), —CH2(Ph), substituted —CH2(Ph), CH2CH2(Ph), substituted —CH2CH2(Ph), —NO2, —CN, —N(R0)2, —NR0C(O)R0, NR0C(O)N(R0)2, NR0CO2R0, —NR0NR0C(O)R0, —NR0NR0C(O)N(R0)2, —NR0NR0C2R0, C(O)C(O)R0, C(O)CH2C(O)R0, —CO2R0, —C(O)R0, —C(O)N(R0)2, —OC(O)N(R0)2, S(O)2R0, —SO2N(R0)2, —S(O)R0, —NR0SO2N(R0)2, —NR0SO2R0, —C(═S)N(R0)2, C(═NH)N(R0)2, (CH2)yNHC(O)R0, and —(CH2)yNHC(O)CH(V—R0)(R0); wherein each R0 is independently selected from hydrogen, a substituted or unsubstituted aliphatic group, an unsubstituted heteroaryl or heterocyclic ring, phenyl (Ph), substituted Ph, O(Ph), substituted —O(Ph), —CH2 (Ph), or substituted —CH2(Ph); y is 0-6; and V is a linker group. Examples of substituents on the aliphatic group or the phenyl ring of R0 include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, and haloalkyl.

An aliphatic group or a non-aromatic heterocyclic ring or a fused aryl or heteroaryl ring may contain one or more substituents. Examples of suitable substituents on any saturated carbon of an aliphatic group or of a non-aromatic heterocyclic ring or a fused aryl or heteroaryl ring include those listed above for the unsaturated carbon of an aryl or heteroaryl group and the following: ═O, ═S, ═NNHR*, ═NN(R*)₂, ═N—, ═NNHC(O)R*, ═NNHCO2(alkyl), ═NNHSO2 (alkyl), or ═NR*, where each R* is independently selected from hydrogen, an unsubstituted aliphatic group, or a substituted aliphatic group. Examples of substituents on the aliphatic group include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, and haloalkyl.

Suitable substituents on the nitrogen of a non-aromatic heterocyclic ring include R+, —N(R+)2, —C(O)R+, —CO2R+, —C(O)C(O)R+, —C(O)CH2C(O)R+, —SO2R+, —SO2N(R+)2, C(═S)N(R+)2, —C(═NH)—N(R+)2, and —NR+SO2R+; wherein each R+ is independently selected from hydrogen, an aliphatic group, a substituted aliphatic group, phenyl (Ph), substituted Ph, —O(Ph), substituted —O(Ph), —CH2(Ph), substituted —CH2(Ph), or an unsubstituted heteroaryl or heterocyclic ring. Examples of substituents on the aliphatic group or the phenyl ring include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, and haloalkyl.

For example, when the C-terminal linker is a lysine amide, the dimer may be illustrated structurally as shown in Formula I, and summarized as shown in Formula II: 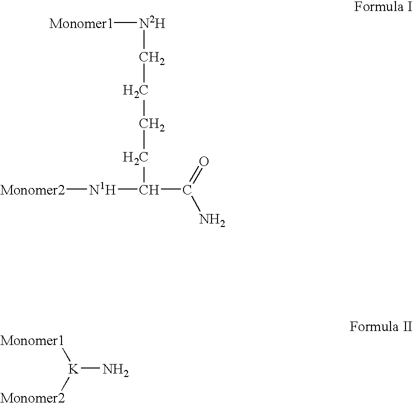
In Formula I, N² represents the nitrogen atom of lysine's ε-amino group and N¹ represents the nitrogen atom of lysine's α-amino group. The dimeric structure can be written as [peptide]2Lys or [peptide]2K to denote a peptide bound to both the α- and ε-amino groups of lysine, or [Ac-peptide]2Lys or [Ac-peptide]2K to denote an N-terminally acetylated peptide bound to both the α- and ε-amino groups of lysine, or [Ac-peptide-Lys*]2-Iminodiacetic-N-(Boc-βAla) or [Ac-peptide-K*]2-Ida to denote a homodimer of an N-terminally acetylated peptide bearing a C-terminal lysine residue where the ε amine of lysine is bound to each of the two carboxyl groups of iminodiacetic acid and where Boc-beta-alanine is covalently bound to the nitrogen atom of iminodiacetic acid via an amide bond, or as [Ac-peptide-K*]-Ida-Boc to denote homodimer of an N-terminally acetylated peptide bearing a C-terminal lysineamide residue where the ε amine of lysine is bound to each of the two carboxyl groups of iminodiacetic acid linker (Ida) and where the amino group of the Ida linker has a Boc group attached.

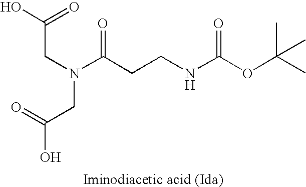

In an optional embodiment, polyethylene glycol (PEG) may serve as the linker that dimerizes two peptide monomers: for example, a single PEG moiety containing two reactive functional groups may be simultaneously attached to the N-termini of both peptide chains of a peptide dimer.

In yet another embodiment, the linker moiety may comprise a molecule containing two carboxylic acids and optionally substituted at one or more available atoms with an additional functional group such as an amine capable of being bound to one or more PEG molecules. Such a molecule can be depicted as:
—CO—(CH₂)n-uX—(CH₂)m-CO—
where n is an integer between zero and 10, m is an integer between one and 10, X is selected from O, S, N(CH₂)pNR1, NCO(CH₂)pNR1, and CHNR1, R1 is selected from H, Boc, Cbz, and p is an integer between 1 and 10. In certain embodiments, one amino group of each of the peptides form an amide bond with the linker. In certain other embodiments, the amino group of the peptide bound to the linker is the epsilon amine of a lysine residue or the alpha amine of the N-terminal residue, or an amino group of the optional spacer molecule. In particularly preferred embodiments, both n and m are one, X is NCO(CH₂)pNR1, p is two, and R1 is Boc. Optionally, the Boc group can be removed to liberate a reactive amine group capable of forming a covalent bond with a suitably activated PEG species such as mPEG-SPA-NHS or mPEG-NPC (Nektar Therapeutics, San Carlos Calif.). Optionally, the linker contains more than one reactive amine capable of being derivatized with a suitably activated PEG species. Optionally, the linker contains one or more reactive amine capable of being derivatized with a suitably activated pharmacokinetic (PK) modifying agent such as a fatty acid, a homing peptide, a transport agent, a cell-penetrating agent, an organ-targeting agent, or a chelating agent.

A peptide monomer or dimer may further comprise one or more spacer moieties.

In one embodiment, the spacer moiety is a C1-12 linking moiety optionally terminated with —NH— linkages or carboxyl (—COOH) groups, and optionally substituted at one or more available carbon atoms with a lower alkyl substituent. In one embodiment, the spacer is R—COOH wherein R is a lower (C1-6) alkyl optionally substituted with a functional group such as a carboxyl group or an amino group that enables binding to another molecular moiety. For example, the spacer may be a glycine (G) residue, or an amino hexanoic acid (Ahx) such as 6-amino hexanoic acid. In other embodiments, the spacer is —NH—R—NH— wherein R is a lower (C1-6) alkyl substituted with a functional group such as a carboxyl group or an amino group that enables binding to another molecular moiety. For example, the spacer may be a lysine (K) residue or a lysine amide (K—NH₂, a lysine residue wherein the carboxyl group has been converted to an amide moiety —CONH₂).

In preferred embodiments, the spacer moiety has the following structure:
—NH—(CH₂)_{α}—[O—(CH₂)_{β}]_{γ}—O_{δ}—(CH₂)_{ε}—Y—
where α, β, γ, δ, and ε are each integers whose values are independently selected. In preferred embodiments, α, β, and ε are each integers whose values are independently selected between one and about six, δ is zero or one, γ is an integer selected between zero and about ten, except that when γ is greater than one, β is two, and Y is selected from NH or CO. In particularly preferred embodiments, α, β, and ε are each equal to two, both γ and δ are equal to 1, and Y is NH. In another particularly preferred embodiment, γ and δ are zero, α and ε together equal five, and Y is CO.

In preferred embodiments, the linker moiety is selected from the following:

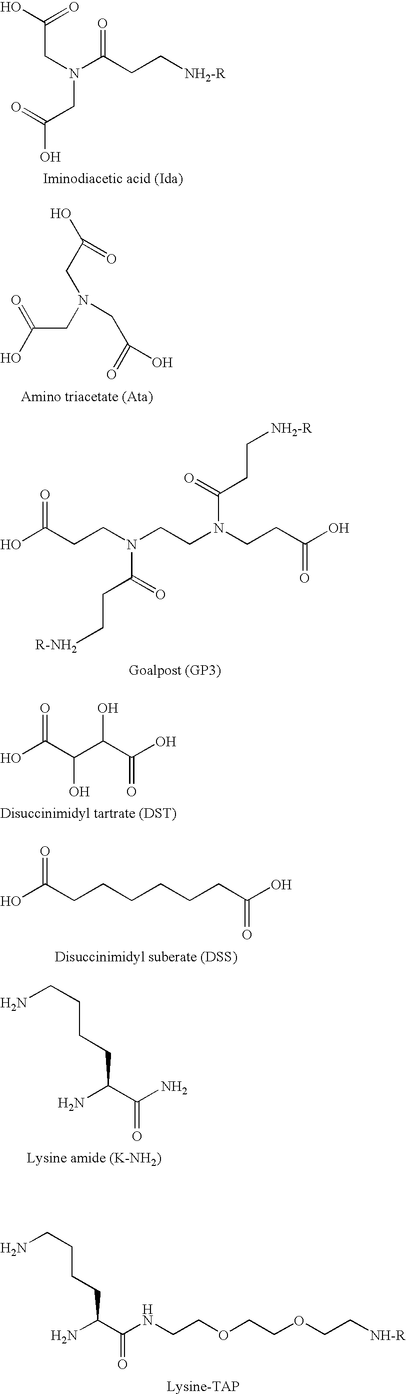

The peptide monomers, dimers, or multimers of the invention may further comprise one or more water soluble polymer moieties. Preferably, these polymers are covalently attached to the peptide compounds of the invention. Preferably, for therapeutic use of the end product preparation, the polymer is pharmaceutically acceptable. One skilled in the art will be able to select the desired polymer based on such considerations as whether the polymer-peptide conjugate will be used therapeutically, and if so, the desired dosage, circulation time, resistance to proteolysis, and other considerations. The water soluble polymer may be, for example, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl-pyrrolidone)polyethylene glycol, propropylene glycol homopolymers, polypropylene oxide/ethylene oxide copolymers, and polyoxyethylated polyols. A preferred water soluble polymer is PEG.

The polymer may be of any molecular weight, and may be branched or unbranched. A preferred PEG for use in the present invention is linear, unbranched PEG having a molecular weight of from about 5 kilodaltons (kDa) to about 60 kDa (the term “about” indicating that in preparations of PEG, some molecules will weigh more, and some less, than the stated molecular weight). More preferably, the PEG has a molecular weight of from about 10 kDa to about 40 kDa, and even more preferably, the PEG has a molecular weight from 20 to 30 kDa. Other sizes may be used, depending on the desired therapeutic profile (e.g., duration of sustained release desired; effects, if any, on biological activity; ease in handling; degree or lack of antigenicity; and other effects of PEG on a therapeutic peptide known to one skilled in the art).

The number of polymer molecules attached may vary; for example, one, two, three, or more water-soluble polymers may be attached to a peptide of the invention. The multiple attached polymers may be the same or different chemical moieties (e.g., PEGs of different molecular weight).

In certain embodiments, PEG may serve as a linker that dimerizes two peptide monomers. In other embodiments, PEG may be attached to at least one terminus (N-terminus or C-terminus) of a peptide monomer or dimer. In other embodiments, PEG may be attached to a spacer moiety of a peptide monomer or dimer. In a preferred embodiment, PEG is attached to the linker moiety of a peptide dimer. Optionally, the linker contains more than one reactive amine capable of being derivatized with a suitably activated PEG species. Optionally, the linker contains one or more reactive amine capable of being derivatized with a suitably activated pharmacokinetic (PK) modifying agent such as a fatty acid, a homing peptide, a transport agent, a cell-penetrating agent, an organ-targeting agent, or a chelating agent.

Other methods for stabilizing peptides known in the art may be used in the present invention. For example, using D-aminoacids, using reduced amide bonds for the peptide backbone, and using non-peptide bonds to link the side chains, including, but not limited to, pyrrolinone and sugar mimetics. The design and synthesis of sugar scaffold peptide mimetics are described by Hirschmann et al. (J. Med. Chem., 1996, 36, 2441-2448), which is incorporated herein by reference in its entirety. Further, pyrrolinone-based peptide mimetics present the peptide pharmacophore on a stable background that has improved bioavailability characteristics (see, for example, Smith et al., J. Am. Chem. Soc. 2000, 122, 11037-11038), which is incorporated herein by reference in its entirety.

Preparation of the Peptide Compounds of the Invention:

Peptide Synthesis

The peptides of the invention may be prepared by classical methods known in the art. These standard methods include exclusive solid phase synthesis, partial solid phase synthesis methods, fragment condensation, classical solution synthesis, and recombinant DNA technology [see, e.g., Merrifield, J. Am. Chem. Soc. 1963 85:2149].

In one embodiment, the peptide monomers of a peptide dimer are synthesized individually and are dimerized or multimerized subsequent to synthesis by reaction with a suitable linker moiety. In preferred embodiments the peptide monomers of a dimer have the same amino acid sequence.

In alternative embodiments, the peptide monomers of a dimer are linked via their C-termini by a linker moiety having two functional groups capable of serving as initiation sites for peptide synthesis and a third functional group (e.g., a carboxyl group or an amino group) that enables binding to another molecular moiety (e.g., as may be present on the surface of a solid support during peptide synthesis or to a pharmacokinetics-modifying agent such as PEG). In this case, the two peptide monomers may be synthesized directly onto two reactive nitrogen groups of the linker moiety in a variation of the solid phase synthesis technique. Such synthesis may be sequential or simultaneous.

Where sequential synthesis of the peptide chains of a dimer onto a linker is to be performed, two amine functional groups on the linker molecule are protected with two different orthogonally removable amine protecting groups. In certain embodiments, the protected diamine is a protected lysine. The protected linker is coupled to a solid support via the linker's third functional group. The first amine protecting group is removed, and the first peptide of the dimer is synthesized on the first deprotected amine moiety. Then the second amine protecting group is removed, and the second peptide of the dimer is synthesized on the second deprotected amine moiety. For example, the first amino moiety of the linker may be protected with Alloc, and the second with Fmoc. In this case, the Fmoc group (but not the Alloc group) may be removed by treatment with a mild base [e.g., 20% piperidine in dimethyl formamide (DMF)], and the first peptide chain synthesized. Thereafter, the Alloc group may be removed with a suitable reagent [e.g., Pd(PPh₃)₄/N-methyl morpholine and chloroform], and the second peptide chain synthesized. This technique may be used to generate dimers wherein the sequences of the two peptide chains are identical or different.

Where simultaneous synthesis of the peptide chains of a dimer onto a linker is to be performed, two amine functional groups of the linker molecule are protected with the same removable amine protecting group. In preferred embodiments, the protected diamine is a protected lysine. The protected linker is coupled to a solid support via the linker's third functional group. In this case the two protected functional groups of the linker molecule are simultaneously deprotected, and the two peptide chains simultaneously synthesized on the deprotected amines. Note that using this technique, the sequences of the peptide chains of the dimer will generally be identical.

One method for peptide synthesis is solid phase synthesis. Solid phase peptide synthesis procedures are well-known in the art [see, e.g., Stewart Solid Phase Peptide Syntheses (Freeman and Co.: San Francisco) 1969; 2002/2003 General Catalog from Novabiochem Corp, San Diego, USA; Goodman Synthesis of Peptides and Peptidomimetics (Houben-Weyl, Stuttgart) 2002]. In solid phase synthesis, synthesis is typically commenced from the C-terminal end of the peptide using an α-amino protected resin. A suitable starting material can be prepared, for instance, by attaching the required α-amino acid to a resin, a hydroxymethyl resin, a polystyrene resin, a benzhydrylamine resin, or the like. One such chloromethylated resin is sold under the trade name BIO-BEADS SX-1 by BioRad Laboratories (Richmond, Calif.). The preparation of the hydroxymethyl resin has been described [Bodonszky, et al. (1966) Chem. Ind. London 38:1597]. The benzhydrylamine (BHA) resin has been described [Pietta and Marshall (1970) Chem. Commun. 650], and the hydrochloride form is commercially available from Beckman Instruments, Inc. (Palo Alto, Calif.). For example, an α-amino protected amino acid may be coupled to a chloromethylated resin with the aid of a cesium bicarbonate catalyst, according to the method described by Gisin (1973) Helv. Chim. Acta 56:1467.

After initial coupling, the α-amino protecting group is removed, for example, using trifluoroacetic acid (TFA) or hydrochloric acid (HCl) or piperidine solutions in organic solvents at room temperature. Thereafter, α-amino protected amino acids are successively coupled to a growing support-bound peptide chain. The α-amino protecting groups are those known to be useful in the art of stepwise synthesis of peptides, including: acyl-type protecting groups, including, but not limited to, acyl, formyl, trifluoroacetyl, and acetyl; aromatic urethane-type protecting groups [e.g., benzyloxycarboyl (Cbz) and substituted Cbz], aliphatic urethane protecting groups [e.g., t-butyloxycarbonyl (Boc), isopropyloxycarbonyl, cyclohexyloxycarbonyl], and alkyl type protecting groups (e.g., benzyl, triphenylmethyl), fluorenylmethyl oxycarbonyl (Fmoc), allyloxycarbonyl (Alloc), and 1-(4,4-dimethyl-2,6-dioxocyclohex-1-ylidene)ethyl (Dde).

The side chain protecting groups (typically ethers, esters, trityl, PMC, and the like) remain intact during coupling and are not split off during the deprotection of the amino-terminus protecting group or during coupling. The side chain protecting group must be removable upon the completion of the synthesis of the final peptide and under reaction conditions that will not alter the target peptide. The side chain protecting groups for Tyr include tetrahydropyranyl, tert-butyl, trityl, benzyl, Cbz, Z-Br—Cbz, and 2,5-dichlorobenzyl. The side chain protecting groups for Asp include t-butyl, benzyl, 2,6-dichlorobenzyl, methyl, ethyl, and cyclohexyl. The side chain protecting groups for Thr and Ser include acetyl, benzoyl, trityl, tetrahydropyranyl, benzyl, 2,6-dichlorobenzyl, and Cbz. The side chain protecting groups for Thr and Ser are benzyl. The side chain protecting groups for Arg include nitro, Tosyl (Tos), Cbz, adamantyloxycarbonyl mesitoylsulfonyl (Mts), 2,2,4,6,7-pentamethyldihydrobenzofurane-5-sulfonyl (Pbf), 4-methoxy-2,3,6-trimethyl-benzenesulfonyl (Mtr), or Boc. The side chain protecting groups for Lys include Cbz, 2-chlorobenzyloxycarbonyl (2-Cl—Cbz), 2-bromobenzyloxycarbonyl (2-Br—Cbz), Tos, or Boc. A number of reviews on protecting groups, familiar to those skilled in the art, include: Meienhofer, J. (1985) in Chemistry and Biochemistry of the Amino Acids (Barrett, G. C., eds.), pp. 297-337, Chapman and Hall, London; Thomas, D. W. and Jones, J. H. Int. J. Peptide Protein Res. 25, 1985, 213-223; Atherton, E. and Sheppard, R. C. (1989) Solid Phase Peptide Synthesis: A Practical Approach Using the Fmoc-Polyamide Technique, IRL press, Oxford; Fields, G. B. and Noble, R. L. Int. J. Peptide Protein Res. 35, 1990, 161-214; and Greene, T. W. and Wuts, P. G. M. (1999) Protective Groups in Organic Chemistry, Third Edition, Wiley-Interscience, New York.

After removal of the α-amino protecting group, the remaining protected amino acids are coupled stepwise in the desired order. Each protected amino acid is generally reacted in about a 3-fold excess using an appropriate carboxyl group activator such as 2-(1H-benzotriazol-1-yl)-1,1,3,3 tetramethyluronium hexafluorophosphate (HBTU) or dicyclohexylcarbodimide (DCC) in solution, for example, in methylene chloride (CH₂Cl₂), N-methylpyrrolidone, dimethyl formamide (DMF), or mixtures thereof.

After the desired amino acid sequence has been completed, the desired peptide is decoupled from the resin support by treatment with a reagent, such as trifluoroacetic acid (TFA) or hydrogen fluoride (HF), which not only cleaves the peptide from the resin, but also cleaves all remaining side chain protecting groups. When a chloromethylated resin is used, hydrogen fluoride treatment results in the formation of the free peptide acids. When the benzhydrylamine resin is used, hydrogen fluoride treatment results directly in the free peptide amide. Alternatively, when the chloromethylated resin is employed, the side chain protected peptide can be decoupled by treatment of the peptide resin with ammonia to give the desired side chain protected amide or with an alkylamine to give a side chain protected alkylamide or dialkylamide. Side chain protection is then removed in the usual fashion by treatment with hydrogen fluoride in the case of Boc-based syhthesis or TFA in the case of Fmoc-based synthesis to give the free amides, alkylamides, or dialkylamides. In preparing the esters of the invention, the resins used to prepare the peptide acids are employed, and the side chain protected peptide is cleaved with base and the appropriate alcohol (e.g., methanol). Side chain protecting groups are then removed in the usual fashion by treatment with hydrogen fluoride to obtain the desired ester.

These procedures can also be used to synthesize peptides in which amino acids other than the 20 naturally occurring, genetically encoded amino acids are substituted at one, two, or more positions of any of the compounds of the invention. D-amino acids and non-naturally occurring synthetic amino acids or non peptide bonds, including but not limited to reduced amides, pyrrolidones and sugar mimetics can also be incorporated into the peptides of the present invention.

Peptide Modifications

One can also modify the amino and/or carboxy termini of the peptide compounds of the invention to produce other compounds of the invention. Amino terminus modifications include methylation (e.g., —NHCH₃ or —N(CH₃)₂), acetylation (e.g., with acetic acid or a halogenated derivative thereof such as α-chloroacetic acid, α-bromoacetic acid, or α-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from the group consisting of alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the peptide compound. In preferred embodiments, the N-terminus is acetylated with acetic acid or acetic anhydride.

Carboxy terminus modifications include replacing the free acid with a carboxamide group or forming a cyclic lactam at the carboxy terminus to introduce structural constraints. One can also cyclize the peptides of the invention, or incorporate a desamino or descarboxy residue at the termini of the peptide, so that there is no terminal amino or carboxyl group, to decrease susceptibility to proteases or to restrict the conformation of the peptide. C-terminal functional groups of the compounds of the present invention include amide, amide lower alkyl, amide di(lower alkyl), lower alkoxy, hydroxy, and carboxy, and the lower ester derivatives thereof, and the pharmaceutically acceptable salts thereof.

One can replace the naturally occurring side chains of the genetically encoded amino acids (or the stereoisomeric D amino acids) with other side chains, for instance with groups such as alkyl, lower (C₁₋₆) alkyl, cyclic 4-, 5-, 6-, to 7-membered alkyl, amide, amide lower alkyl amide di(lower alkyl), lower alkoxy, hydroxy, carboxy and the lower ester derivatives thereof, and with 4-, 5-, 6-, to 7-membered heterocyclic. In particular, proline analogues in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

One can also readily modify peptides by phosphorylation, and other methods [e.g., as described in Hruby, et al. (1990) Biochem J. 268:249-262].

The peptide compounds of the invention also serve as structural models for non-peptidic compounds with similar biological activity. Those of skill in the art recognize that a variety of techniques are available for constructing compounds with the same or similar desired biological activity as the lead peptide compound, but with more favorable activity than the lead with respect to solubility, stability, and susceptibility to hydrolysis and proteolysis [See, Morgan and Gainor (1989) Ann. Rep. Med. Chem. 24:243-252]. These techniques include, but are not limited to, replacing the peptide backbone with a backbone composed of phosphonates, amidates, carbamates, sulfonamides, secondary amines, and N-methylamino acids.

Anti-proliferative activity is evaluated in situ by testing the ability of the fragments to inhibit the proliferation of new blood vessel cells, referred to herein as the inhibition of angiogenesis. A suitable assay is the chick embryo chorioallantoic membrane (CAM) assay described by Crum et al., Science 230:1375 (1985) and described in U.S. Pat. No. 5,001,116, which is incorporated by reference herein. The CAM assay is briefly described as follows. Fertilized chick embryos are removed from their shell on day 3 or 4, and a methylcellulose disc containing the fragment of interest is implanted on the chorioallantoic membrane. The embryos are examined 48 hours later and, if a clear avascular zone appears around the methylcellulose disc, the diameter of that zone is measured. The larger the diameter of the zone, the greater the anti-angiogenic activity. An additional assay used to assess antiangiogenic activity is the Matrigel assay and is described by Passaniti et al. (Passaniti et al. Lab. Invest. 67(4):519-28 (1992)).

Formulations

The synthetic protein, peptide, or protein fragment, can be prepared in a physiologically acceptable formulation, such as in a pharmaceutically acceptable carrier, using known techniques. For example, the protein, peptide or protein fragment is combined with a pharmaceutically acceptable excipient to form a therapeutic composition.

The composition may be in the form of a solid, liquid or aerosol. Examples of solid compositions include pills, creams, and implantable dosage units. Pills may be administered orally. Therapeutic creams may be administered topically. Implantable dosage units may be administered locally, for example, at a tumor site, or may be implanted for systematic release of the therapeutic composition, for example, subcutaneously. Examples of liquid compositions include formulations adapted for injection subcutaneously, intravenously, intra-arterially, and formulations for topical and intraocular administration. Examples of aerosol formulations include inhaler formulations for administration via the lungs.

The composition may be administered by standard routes of administration. In general, the composition may be administered by topical, oral, rectal, vaginal, nasal or parenteral (for example, intravenous, subcutaneous, or intramuscular) routes. In addition, the composition may be incorporated into sustained release matrices such as biodegradable polymers, the polymers being implanted in the vicinity of where delivery is desired, for example, at the site of a tumor. Biodegradable and other polymers can also be implanted so as to enable systemic delivery, for example from a s.c or i.m. implantation site The method includes administration of a single dose, administration of repeated doses at predetermined time intervals, and sustained administration for a predetermined period of time.

A sustained release matrix, as used herein, is a matrix made of materials, usually polymers which are degradable by enzymatic or acid/base hydrolysis or by dissolution. Once inserted into the body, the matrix is acted upon by enzymes and body fluids. The sustained release matrix desirably is chosen by biocompatible materials such as liposomes, polylactides (polylactide acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The dosage of the composition will depend on the condition being treated, the particular composition used, and other clinical factors such as weight and condition of the patient, and the route of administration.

The composition may be administered in combination with other compositions and procedures for the treatment of diseases. For example, unwanted cell proliferation may be treated conventionally with surgery, radiation or an approved anticancer drug in combination with the administration of the composition, and additional doses of the composition may be subsequently administered to the patient to stabilize and inhibit the growth of any residual unwanted cell proliferation.

Diseases and Conditions to be Treated

The invention can be used to treat any disease characterized by abnormal cell mitosis and/or abnormal angiogenesis in humans or animals. Such diseases include, but are not limited to, abnormal stimulation of endothelial cells (e.g., atherosclerosis); solid tumors; blood-borne tumors, such as leukemias; tumor metastasis; benign tumors, for example, hemangiomas, acoustic neuromas, neurofibromas, trachomas, and pyogenic granulomas; vascular malfunctions; abnormal wound healing; inflammatory and immune disorders; Behcet's disease; gout or gouty arthritis; abnormal angiogenesis accompanying: rheumatoid arthritis; skin diseases, such as psoriasis; diabetic retinopathy, and other ocular angiogenic diseases, such as retinopathy of prematurity (retrolental fibroplasia), macular degeneration, corneal graft rejection, neovascular glaucoma; liver diseases and Oster Webber Syndrome (Osler-Weber-Rendu disease).

Diseases associated with neovascularization can be treated in humans and animals according to the present invention. Such diseases include, but are not limited to, ocular neovascular disease, diabetic retinopathy, retinopathy of prematurity, corneal graft rejection, neovascular glaucoma and retrolental fibroplasias, epidemic keratoconjunctivitis, Vitamin A deficiency, contact lens overwear, atopic keratitis, superior limbic keratitis, pterygium keratitis sicca, Sjögren's syndrome, acne rosacea, phylectenulosis, syphilis, Mycobacterial infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, scleritis, Steven-Johnson disease, pemphigoid, radial keratotomy, and corneal graph rejection.

Other diseases associated with neovascularization can be treated according to the present invention. Such diseases include, but are not limited to, sickle cell anemia, sarcoid, pseudoxanthoma elasticum, Paget's disease, vein occlusion, artery occlusion, carotid obstructive disease, chronic uveitis/vitritis, Lyme's disease, systemic lupus erythematosis, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, and post-laser complications. Other diseases include, but are not limited to, diseases associated with rubeosis (neovascularization of the iris and the angle) and diseases caused by the abnormal proliferation of fibrovascular or fibrous tissue including all forms of proliferative vitreoretinopathy, whether or not associated with diabetes.

The present invention may also be used to treat angiogenesis-dependent cancers including, but not limited to, any one or combination of rhabdomyosarcoma, retinoblastoma, Ewing sarcoma, neuroblastoma, and osteosarcoma. Other angiogenesis-dependent cancers treatable with the present invention include, but are not limited to, breast cancer, prostrate cancer, renal cell cancer, brain cancer, ovarian cancer, colon cancer, bladder cancer, pancreatic cancer, stomach cancer, esophageal cancer, cutaneous melanoma, liver cancer, small cell and non-small cell lung cancer, testicular cancer, kidney cancer, bladder cancer, cervical cancer, lymphoma, parathyroid cancer, penile cancer, rectal cancer, small intestine cancer, thyroid cancer, uterine cancer, Hodgkin's lymphoma, non-Hodgkin's lymphoma, lip cancer, oral cancer, skin cancer, leukemia or multiple myeloma.

As mentioned above, another disease that can be treated according to the present invention is rheumatoid arthritis. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Other diseases that can be treated according to the present invention are hereditary hemorrhagic telangiectasia, osteoarthritis, chronic inflammation, Crohn's disease, ulcerative colitis, Bartonellosis, inflammatory or immune mediated bowel disease and acquired immune deficiency syndrome.

The present invention can be used to treat eye conditions in humans or animals, wherein the eye conditions include, but are not limited to, ocular neovascular disease, diabetic retinopathy, retinopathy of prematurity, macular degeneration, corneal graft rejection, neovascular glaucoma, retrolental fibroplasias, epidemic keratoconjunctivitis, contact lens overwear, atopic keratitis, superior limbic keratitis, pterygium keratitis sicca, myopia, chronic retinal detachment, optic pits, Terrien's marginal degeneration, hyperviscosity syndromes, chronic uveitis, chronic vitritis, presumed ocular histoplasmosis, retinitis, choroiditis, proliferative vitreoretinopathy, scleritis, Eales' disease, Best's disease, trachoma, or post-laser complications.

The present invention can be used to treat inflammatory or immune mediated diseases in humans or animals, wherein the inflammatory or immune mediated diseases include, but are not limited to, rheumatoid arthritis, osteoarthritis, ulcerative colitis, Crohn's disease, Mooren's ulcer, arthritis, sarcoidosis, inflammatory or immune mediated bowel disease, systemic lupus, Wegener's syndrome, Stevens-Johnson disease, Behcet's disease, pemphigoid, Lyme's disease, asthma or acquired immune deficiency syndrome.

The present invention can be used to treat infectious diseases in humans or animals, wherein the infectious diseases include, but are not limited to, syphilis, a bacterial infection, a Mycobacterial infection, a bacterial ulcer, a fungal ulcer, a Herpes simplex infection, a Herpes zoster infection, a protozoan infection, a Bartonellosis infection, or toxoplasmosis.

The present invention can be used to treat blood or blood vessel diseases in humans or animals, wherein the blood or blood vessel diseases include, but are not limited to, vein occlusion, artery occlusion, carotid obstructive disease, polyarteritis, atherosclerosis, Osler-Weber-Rendu disease, sickle cell anemia, leukemia, acute or chronic neoplastic disease of the bone marrow, hemangiomas, hereditary hemorrhagic telangiectasia, disease of the bone marrow, anemia, impaired blood clotting or enlargement of the lymph nodes, liver, or spleen. The present invention can also be used to treat chronic neoplastic disease of the bone marrow, wherein those diseases include, but are not limited to, multiple myeloma and myelo dysplastic syndrome.

The present invention can be used to treat skin conditions in a humans or an animals, wherein the skin conditions include, but are not limited to, abnormal wound healing, acne rosacea, chemical burns of the skin, dermatitis or psoriasis.

In addition, the invention can be used to treat a variety of post-menopausal symptoms, osteoporosis, cardiovascular disease, myocardial angiogenesis, plaque neovascularization, hemophiliac joints, angiofibroma, wound granulation, intestinal adhesions, scleroderma, hypertrophic scars; i.e., keloids. They are also useful in the treatment of diseases that have angiogenesis as a pathologic consequence, such as cat scratch disease, and Helicobacter pylori ulcers. The invention can also be used to treat Alzheimer's disease, to reduce the incidence of stroke, and as an alternative to prior estrogen replacement therapies.

Additionally, the compounds of the present invention can be used to treat endometriosis. Endometriosis is the abnormal growth of endometrial cells; the cells that line the uterus that are shed monthly in the menstrual process. Wayward endometrial cells can position themselves in the lower abdomen on areas such as the cul-de-sac, the recto-vaginal septum, the stomach, the fallopian tubes, the ovaries, and the bladder. During menstruation, the normal uterine lining is sloughed off and expelled through the vagina, but transplanted endometrial tissue has no means of exiting the body; instead the endometrial tissue and cells adhere and grow where positioned. The results are internal bleeding, inflammation, and scarring. One of the serious consequences of endometrial scarring is infertility. The endometrial growths are generally not malignant or cancerous. Among other complications, the growths can rupture and can spread the endometriosis to new areas of the lower abdomen. Endometriosis is a progressive disease. The growths or lesions are first seen as clear vesicles, then become red, and finally progress to black lesions over a period of seven to ten years.

### DEFINITIONS

The terms “a”, “an” and “the” as used herein are defined to mean one or more and include the plural unless the context is inappropriate.

The term “peptides” describes chains of amino acids (typically L-amino acids) whose alpha carbons are linked through peptide bonds formed by a condensation reaction between the carboxyl group of the alpha carbon of one amino acid and the amino group of the alpha carbon of another amino acid. In naturally occurring peptides, in most cases, the terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free alpha-amino group on the amino acid at the amino terminal of the peptide, or to the alpha-amino group (amido group when participating in a peptide bond) of an amino acid at any other location within the peptide. Similarly, the term “carboxy terminus” (abbreviated C-terminus) refers to the free carboxyl group on the amino acid at the carboxy terminus of a peptide, or to the carboxyl group of an amino acid at any other location within the peptide.

Typically, the amino acids making up a peptide are numbered in order, starting at the amino terminal and increasing in the direction toward the carboxy terminal of the peptide. Thus, when one amino acid is said to “follow” another, that amino acid is positioned closer to the carboxy terminal of the peptide than the preceding amino acid. Here, naturally occurring amino acids are represented in the text by the commonly used one letter codes (e.g. G=glycine).

The term “residue” is used herein to refer to an amino acid

(D or L enantiomer) that is incorporated into a peptide by an amide bond. As such, the amino acid may be a naturally occurring amino acid or, unless otherwise limited, may encompass known analogs of natural amino acids that function in a manner similar to the naturally occurring amino acids (i.e., amino acid mimetics). Moreover, an amide bond mimetic includes peptide backbone modifications well known to those skilled in the art.

Furthermore, one skilled in the art will recognize that, as mentioned above, individual substitutions, deletions or additions which alter, add or delete a single amino acid or several amino acids in a sequence are conservatively modified variations where the alterations result in the substitution of an amino acid with a chemically similar amino acid. Conservative substitution tables providing functionally similar amino acids are well known in the art. The following six groups each contain examples of amino acids that are frequently considered as conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K); Histidine (H);

5) Isoleucine (I), Leucine (L), Methionine (M), Valine (V);

6) Phenylalanine (F), Tyrosine (Y), Tryptophan (W).

One skilled in the art will recognize that unnatural amino acids may be substituted for the amino acids of this invention. For example, the term “basic amino acid”, in addition to arginine, lysine, and histidine noted above as group 4, may also include any amino acid with a side chain functional group capable of displaying ionic interactions, such as ornithine, substituted arginine residues, substituted lysine residues, or other amino acids containing amino, amidines, or guanidinyl groups in their side chains. The term “hydrophobic amino acid”, in addition to isoleucine, leucine, methionine, and valine noted above as group 5, may also include any amino acid with a side chain containing a non-hydrophilic group, such as those containing lower alkyls, branched aliphatics, aromatics, and the like. The term “aromatic amino acid”, in addition to phenylalanine, tyrosine, and tryptophan noted above as group 6, may also include any amino acid with a side chain capable of forming π-π stacking interactions, such as those containing cyclic conjugated rings, substituted phenyl rings, pyridyl rings, fused-ring aromatics, and the like. The term “polar amino acid”, in addition to asparagine and glutamine noted above as group 3, may also include any amino acid with a side chain capable of displaying polar-polar interactions often described as hydrogen-bond donors or acceptors, such as esters, amides, ureas, sulfonamides, sulfoxides, sulfones, and the like. The term “small amino acid”, in addition to alanine, serine, and threonine noted above as group 1, may also include any amino acid with a side chain lacking a strong steric interaction, such as aminobutyric acid, or aminoisobutyric acid. The term “acidic amino acid”, in addition to aspartic and glutamic acids noted above as group 2, may also include any amino acids containing a side chain functional group capable of protonating another functional group, such as carboxylic acids, sulfonic acids, sulfonamide, sulfonylureas, tetrazoles, and the like. One skilled in the art will recognize that some amino acids can be included in more than one group; for example the amino acids serine and threonine are commonly grouped into both the polar amino acid and small amino acid groups, and amino acids such as alanine and proline are commonly grouped into both the small and hydrophilic amino acid groups.

Typically, the isolated, antiproliferative peptides described herein are at least about 80% pure, usually at least about 90%, and preferably at least about 95% as measured by HPLC.

When peptides are relatively short in length (i.e., less than about 50 amino acids), they are often synthesized using chemical peptide synthesis techniques. Solid phase synthesis is a method in which the C-terminal amino acid of the sequence is attached to an insoluble support followed by sequential addition of the remaining amino acids in the sequence. This is a preferred method for the chemical synthesis of the peptides described herein. Techniques for solid phase synthesis are known to those skilled in the art.

Short peptides and related amides can also by synthesized efficiently by solution phase coupling chemistry. Amino acids and related molecules, with the appropriate protection groups, are coupled in solution to yield amides and peptides. Coupling reagents for forming amide bonds include, but are not limited to, 1,3-dicyclohexyl carbodiimide, 1-hydroxybenzotriazole, 2-(H-benzotriazole-1-yl)-1,1,3,3-tetramethyluronium tetrafluoroborate, or carbonyl diimidizole.

As employed herein, the phrase “biological activity” refers to the functionality, reactivity, and specificity of compounds that are derived from biological systems or those compounds that are reactive to them, or other compounds that mimic the functionality, reactivity, and specificity of these compounds. Examples of suitable biologically active compounds include, but are not limited to, enzymes, antibodies, antigens and proteins.

The term “bodily fluid,” as used herein, includes, but is not limited to, saliva, gingival secretions, cerebrospinal fluid, gastrointestinal fluid, mucous, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, vitreal fluid, and nasal secretions.

The compositions and methods are further illustrated by the following non-limiting examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Synthesis of a Dimeric Peptide on TentaGel-Rink Resin

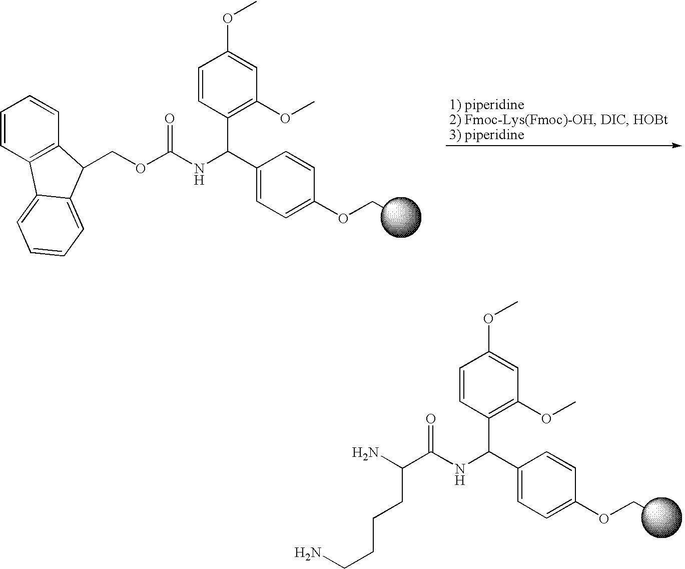

TentaGel-Rink resin: The synthesis was carried out on an Aaptec model 90 solid-phase peptide synthesizer. Step 1: TentaGel-Rink resin (25.0 g, 0.2 mmol/g from Rapp Polymere, Germany) was treated with 150 mL of a solution of 20% piperidine in DMF (1×2 min, 1×25 min). Step 2: The resin was washed (DMF, DCM, MeOH, DMF, 150 mL). Step 3: The resin was treated with an activated solution of Fmoc-AA-OH (prepared from 4 eq. amino acid and 4 eq. HOBt in DMF (0.25 M), followed by the addition of 4 eq. of DIC in DMF, 0.25 M) and allowed to mix for 2 hours. Step 4: The resin was treated for a second time with an activated solution of Fmoc-AA-OH (prepared from 4 eq. amino acid and 4 eq. HOBt in DMF (0.25 M), followed by the addition of 4 eq. of DIC in DMF, 0.25 M) and allowed to mix for 2 hours. Step 5: The resin was washed (DCM, MeOH, DMF, 150 mL) and steps 1-5 were repeated until the desired dimeric peptide sequence was obtained.

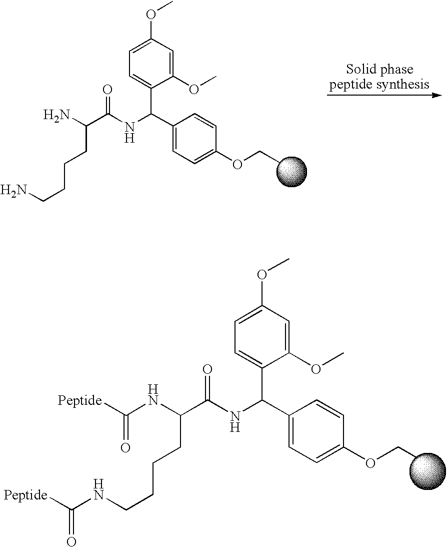

TentaGel-Rink-Lys[Peptide]2: As described above, the resin from above was subjected to repeated cycles of Fmoc-amino acid couplings with DIC/HOBt activation and Fmoc removal with piperidine to build both peptide chains simultaneously. After the final Fmoc removal, the terminal amine groups were acylated by treating the resin with a solution of 10% acetic anhydride, 20% pyridine in THF (150 mL) for 40 minutes, followed by washing (DCM, MeOH, DMF, 150 mL). The resin was dried to yield the protected peptide resin.

Lys[Peptide]2: Once the synthesis was complete, the protected peptide resin from above was subjected to 500 mL of a cocktail of TFA/TIPS/phenol/H₂O (88:2:5:5) with agitation for 3 hours. The mixture was then filtered to remove the resin and washed twice with 100 mL of additional TFA cocktail. The dimeric peptide was then precipitated from the TFA with cold diethyl ether (10 fold excess by volume) and centrifugation. The precipitated peptide was washed twice with diethyl ether and dried yielding 9 g deprotected peptide (amino acid side chain protecting groups were removed simultaneously with peptide cleavage from the resin). The peptide was then purified by C18 reversed phase HPLC using ACN/water containing 0.1% TFA to yield pure dimeric peptide (2 g, 0.51 mmol).

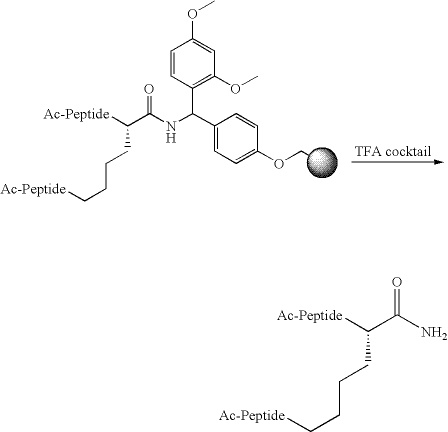

### Example 2

### Synthesis of a Linear Peptide on TentaGel-Rink Resin

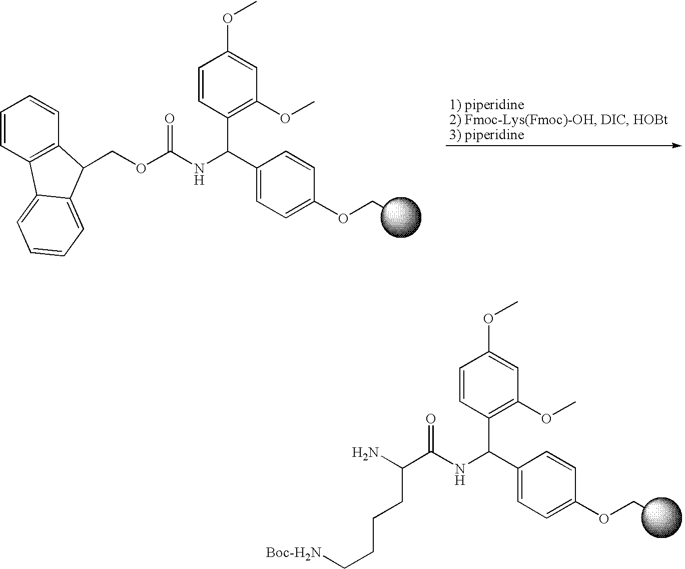

TentaGel-Rink resin: The synthesis was carried out on an Aaptec model 90 solid-phase peptide synthesizer. Step 1: TentaGel-Rink resin (25.0 g, 0.2 mmol/g from Rapp Polymere, Germany) was treated with 150 mL of a solution of 20% piperidine in DMF (1×2 min, 1×25 min). Step 2: The resin was washed (DMF, DCM, MeOH, DMF, 150 mL). Step 3: The resin was treated with an activated solution of Fmoc-AA-OH (prepared from 4 eq. amino acid and 4 eq. HOBt in DMF (0.25 M), followed by the addition of 4 eq. of DIC in DMF, 0.25 M) and allowed to mix for 2 hours. Step 4: The resin was washed (DCM, MeOH, DMF, 150 mL) and steps 1-4 were repeated until the desired linear peptide sequence was obtained. After the final Fmoc removal, the terminal amine groups were acylated by treating the resin with a solution of 10% acetic anhydride, 20% pyridine in THF (150 mL) for 40 minutes, followed by washing (DCM, MeOH, DMF, 150 mL). The resin was dried to yield the protected peptide resin.

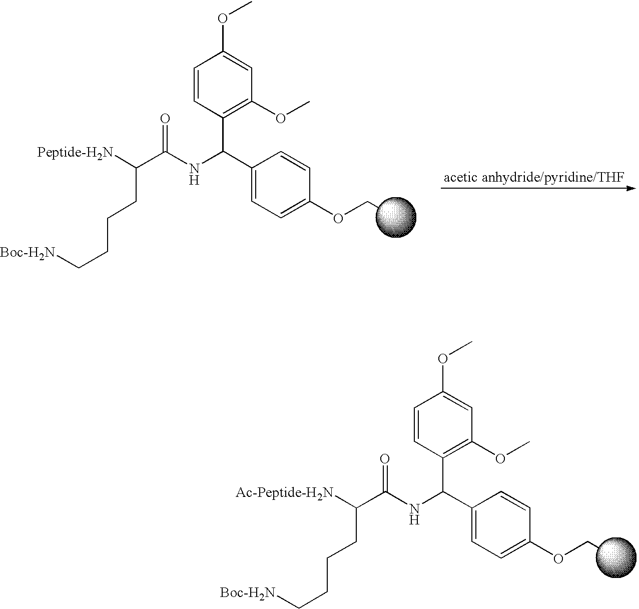

Linear peptide (monomer): Once the synthesis was complete, the protected peptide resin from above was subjected to 500 mL of a cocktail of TFA/TIPS/phenol/H₂O (88:2:5:5) with agitation for 3 hours. The mixture was then filtered to remove the resin and washed twice with 100 mL of additional TFA cocktail. The dimeric peptide was then precipitated from the TFA with cold diethyl ether (10 fold excess by volume) and centrifugation. The precipitated peptide was washed twice with diethyl ether and dried yielding 11 g crude deprotected peptide (amino acid side chain protecting groups were removed simultaneously with peptide cleavage from the resin). The peptide was then purifed by C18 reversed phase HPLC using ACN/water containing 0.1% TFA to yield pure linear monomer (5 g, 2.84 mmol).

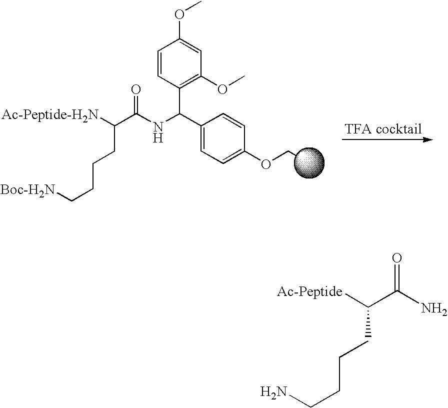

### Example 3

### Dimerization and PEGylation Using a Trifunctional Amine Linker and a Trifunctional Molecule

A first trifunctional molecule having the structure 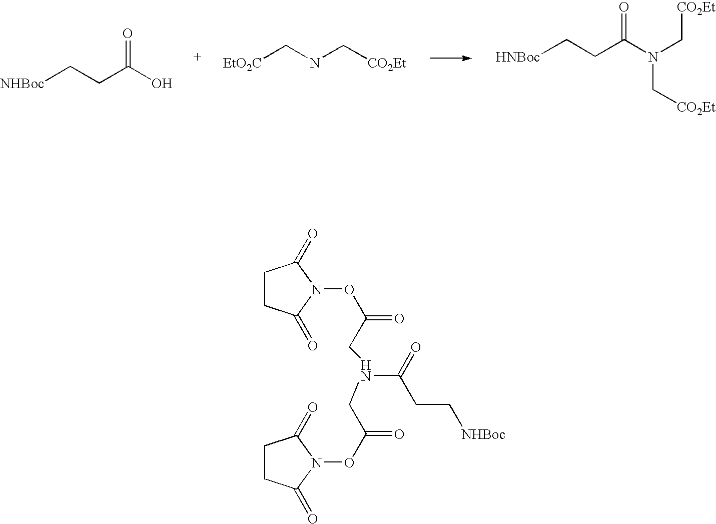
was made according to the following:

To a solution of Boc-βAla-OH (10.0 g, 52.8 mmol) (Boc=tert-butoxycarbonyl) and diethyl iminodiacetate (10.0 g, 52.8 mmol) in 200 mL of DCM at 0° C. was added DCC (10.5 g, 50.9 mmol) over 5 minutes. A white precipitate formed within 2 minutes. The reaction mixture was allowed to warm to room temperature and was stirred for 24 hours. The urea was filtered off with a sintered filter (medium porosity) and the solvent removed under reduced pressure. The residue was taken up in 500 mL of EtOAc (EtOAc=ethyl acetate), filtered as above, and transferred to a separatory funnel. The organic phase was washed (sat. NaHCO₃, brine, 1 N HCl, brine), dried (MgSO₄), filtered, and dried to yield a colorless oil. The oil solidified to yield a white crystalline solid within 10 minutes.

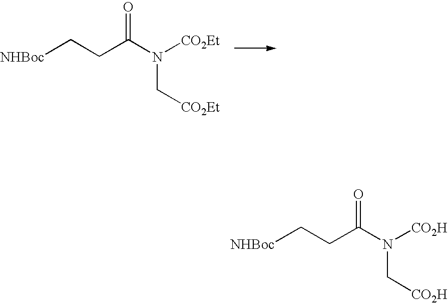

The crude diester was taken up in 75 mL of THF (THF=tetrahydrofuran) and 75 mL of MeOH (MeOH=methanol) and 50 mL of water was added. To this solution was added a solution of KOH (KOH=potassium hydroxide) (8.6 g, 153 mmol) in 25 mL of water. The reaction mixture turned light yellow in color. After stirring for 12 hours (pH was still ˜12), the organic solvent was removed on a rotary evaporator and the resultant slurry partitioned between Et2O (Et2O=Diethyl ether) and saturated NaHCO₃. The combined aqueous phase was acidified to pH 1, saturated with NaCl, and extracted with EtOAc. The EtOAc phase was washed (brine), dried (MgSO₄), and concentrated to yield 13.97 g of product as a white solid (90.2% for 2 steps).

To a solution of diacid (1.00 g, 3.29 mmol) and hydroxysuccinimide (0.945 g, 8.21 mmol) in 50 mL of ACN was added DCC (1.36 g, 6.59 mmol) over 5 minutes. A white precipitate formed immediately. The reaction mixture was stirred 22 hours and was filtered to remove the DCC urea. The solvent was removed under reduced pressure and the residue taken up in EtOAc (250 mL) and transferred to a separatory funnel. The organic phase was washed (saturated NaHCO₃, brine, 1 N HCl, brine), dried (MgSO₄), and concentrated to afford a white solid. The solid was taken up in 75 mL of ACN, filtered, and concentrated to yield 1.28 g of product as a white solid (78.2% yield).

Step 1—Coupling of the Trifunctional Linker to the Peptide Monomers:

For coupling to the linker, 2 eq. peptide (715 mg, 0.406 mmol) was mixed with 1 eq. of trifunctional linker (101 mg, 0.203 mmol) in dry DMF (30 mL) to give a clear solution, and 10 eq. of DIEA (0.7 mL, 4.05 mmol) was added after 2 minutes. The mixture was stirred at ambient temperature for 3 hours and monitored by analytical C-18 reverse phase HPLC. Once the reaction was complete, the peptide dimer was precipitated with diethyl ether, followed by purification with C18 preparative reversed phase HPLC using ACN/water containing 0.1% TFA to yield pure peptide dimer (536 mg, 0.15 mmol). The structure of the peptide dimer was confirmed by LC mass spectral analysis (MW 3690.53, calc.; MW 3691, det.). 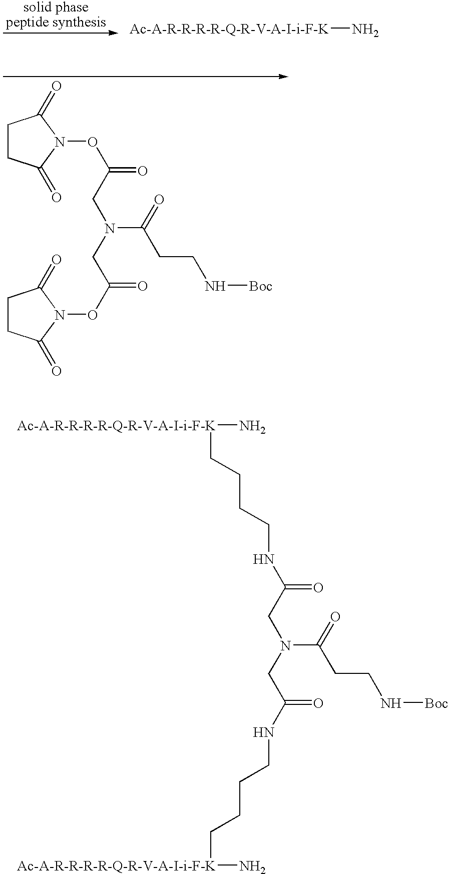
Step 2—PEGylation of the Peptide Dimer:
PEGylation Via a Carbamate Bond:

The Boc group on the above linker was removed upon treatment with 1:1 TFA:DCM for 30 minutes. The peptide was isolated by precipitation from diethyl ether and drying under reduced pressure. The peptide dimer (240 mg, 0.065 mmol) and the PEG species (1560 mg 30 kDa PEG, mPEG-NPC manufactured by NOF Corp., Japan, available through Nektar Therapeutics, U.S., (formerly Shearwater Corp.)) were mixed in a 1:2 molar ratio in 70% DMSO/30% ACN (8 mL) to afford a clear solution. After 5 minutes, 40 eq. of DIEA (453 μL, 2.6 mmol) was added to above solution. The mixture was stirred at ambient temperature 14 hours and monitored by analytical C-18 reverse phase HPLC. Once the pegylation reaction was complete, the pegylated peptide was precipitated with diethyl ether, followed by purification with C18 preparative reversed phase HPLC using ACN/water containing 0.1% TFA to yield pure peptide-PEG30 kDa (1.08 g, 0.032 mmol). The structure of the PEGylated peptide was confirmed by MALDI mass spectral analysis (MW 33704.88 calc.; 33706, det.).

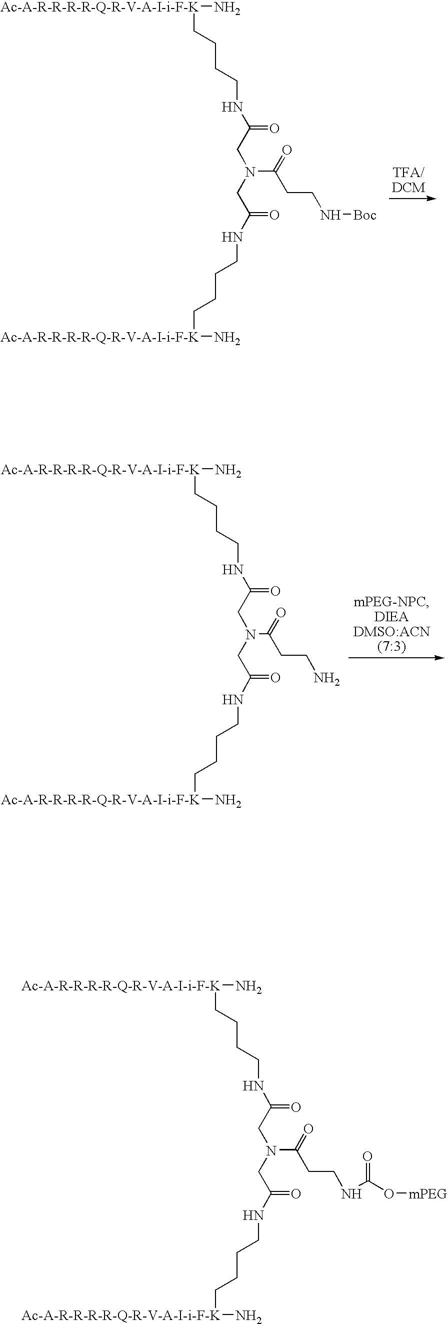

### Example 4

### Proliferation Assay of Endothelial Cells

HUVECs were routinely cultured to near confluency in EGM media (Clontech, Mountain View, Calif.). The cells were trypsinized and plated in a 96-well plate at 5,000 cells per well per 100 μl EBM supplemented with 2% FCS and antibiotics. The cells were allowed to adhere to the plate for at least 12 hours. Then, bFGF at 10 ng/ml or VEGF at 5 ng/ml, and various concentrations of peptide, were added to the wells. The cells were cultured for 48 hours at 37° C. in a 5% CO₂ atmosphere. Cell proliferation was determined using a bromo-deoxyuridine (BrdU) incorporation method as described by the manufacturer. Inhibition of cell proliferation was assessed by BrdU incorporation.

The activities of representative peptides of this invention for their ability to inhibit HUVEC proliferation are listed above in Table 1.

### Example 5

### Proliferation Assay of Tumor Cells

The antiproliferative activity of selected peptides of the present invention was assessed on several tumor cell lines. Mouse Lewis lung carcinoma (LLC) cells were routinely cultured to near confluency in DMEM media with 10% FCS added. The cells were trypsinized and plated in a 96-well plate at 5,000 cells per well per 100 μl media supplemented with 2% FCS and antibiotics. MDA-MB-231 cells were routinely cultured to near confluency in advanced DMEM media supplemented with 10% FCS. The cells were allowed to adhere to the plate for at least 12 hours. Then, whole media and various concentrations of peptide, were added to the wells. The cells were cultured for 48 hours at 37° C. in a 5% CO₂ atmosphere. Cell proliferation was determined using a bromo-deoxyuridine (BrdU) incorporation method as described by the manufacturer. Inhibition of cell proliferation, was assessed by BrdU incorporation.

The ability of representative peptides of this invention for their ability to inhibit various tumor cells line proliferation are listed below in Table 2.

TABLE 2

| Peptide | Inhibitory Activity (1C50 uM) | | |
|---|---|---|---|
| SegID | HUVEC | MDA-MB-231 | LLC |
| 132 | <10 | <10 | <10 |
| 167 | <10 | <10 | <10 |
| 214 | >30 | >30 | >30 |
| 228 | <10 | <10 | <10 |
| 240 | <10 | <10 | <10 |
| 244 | <10 | <10 | <10 |
| 250 | <10 | 10-30 | 10-30 |
| 254 | <10 | <10 | <10 |
| 267 | <10 | <10 | <10 |
| 272 | <10 | >30 | >30 |
| 279 | <10 | <10 | <10 |
| 315 | <10 | <10 | >30 |
| 322 | <10 | 10 | <10 |
| 331 | <10 | <10 | <10 |
| 333 | <10 | 10 | <10 |
| 346 | >30 | >30 | >30 |
| 375 | <10 | <10 | <10 |
| 376 | <10 | <10 | >30 |
| 381 | <10 | 10-30 | >30 |
| 385 | <10 | 10-30 | >30 |

### Example 6

### Antiangiogenic Activity in a Murine Matrigel Angiogenesis Model

In order to assess the effect of the peptides in this invention on angiogenesis, an additional assay was used to assess antiangiogenic activity. The assay used was the murine Matrigel plug assay, methods for which are described by Passaniti et al. (Lab. Invest. 67(4):519-28 (1992)).

Briefly, Matrigel (BD Biosciences, San Jose, Calif.) is mixed with 0.5 ug/ml of bFGF. 0.5 ml of this Matrigel/bFGF mixture is then injected subcutaneously on the ventral abdominal surface at the level of the xiphoid process of C57B1/6 mice. Matrigel plugs, injected in absence of bFGF addition, are used as negative controls. Treatment with various peptides is initiated 12-18 hours later. Animals are treated by i.p. administration of peptides, dosed at 0.2 mg/d of peptide dimers, and 1.4 mg/d of Pegylated peptide dimers. After 10-12 days of treatment, animals are euthanized, and the Matrigel plugs are surgically removed, and cleaned of mouse tissue, fascia, and fat. The plugs are individually homogenized in 1 ml of distilled water using a tissue grinder, and the homogenate centrifuged for 10 minutes at 1500 g. After centrifugation, supernatant for each plug is assayed for the presence of Hemoglobin using a Serum Hemoglobin analysis kit (Sigma Diagnostics, St. Louis, Mo.). Angiogenesis is defined as the fold increase of hemoglobin in the buffer control treated mouse Matrigel plugs, above the amount of hemoglobin in negative (−bFGF) control Matrigel plugs. For experimental peptide treated animals, the percent inhibition is defined as the inhibition seen when comparing positive control plugs (+bFGF/Buffer treatment) with peptide treated plugs (+bFGF/Peptide treatment).

The antiangiogenic activity of a number of representative peptides is presented below in Table 3.

TABLE 3

| | Matrigel Model |
|---|---|
| Seq ID | % Inhibition |
| 132 | 11.0 |
| 167 | 42.0 |
| 375 | 46.0 |
| 228 | 55.0 |
| 376 | 42.0 |
| 267 | 36.0 |
| 279 | 56.0 |
| 315 | 52.0 |
| 381 | 51.0 |
| 331 | 44.0 |
| 254 | 60.2 |

It should be understood, of course, that the foregoing relates only to preferred embodiments of the present invention and that numerous modifications or alterations may be made therein without departing from the spirit and the scope of the invention as set forth in the appended claims.

We claim:

1. A composition comprising one or more peptides:
wherein the peptides comprise 8-40 amino acid residues in length, and the amino acid residue sequence of the residues comprises:
X₁—(X_{A})_{N}—X_{B}—X_{C}—(X_{A})_{N}—X_{D}—(X_{A})_{N}—X_{E}—X_{F}—X_{G}—X_{H},
wherein X₁ comprises a residue selected from the group consisting of any amino acid;
wherein X_{A} comprises a number (N) of residues individually selected from the group consisting of basic amino acids and A;
wherein N is an integer selected from the group consisting of 1, 2, 3, 4, 5, 6, 7, 8, 9 and 10;
wherein X_{B} comprises a residue selected from the group consisting of any amino acid;
wherein X_{C} comprises a residue selected from the group consisting of A, Y, Q, W, V, T, P, N, M, I, F, G, and E;
wherein X_{D} comprises a residue selected from the group consisting of V, a hydrophobic amino acid, or a valence bond;
wherein X_{E} comprises a residue selected from the group consisting of I, V, L, a hydrophobic amino acid or a valence bond;
wherein X_{F} comprises a residue selected from the group consisting of I, V, L, F, a hydrophobic amino acid, an aromatic amino acid or a valence bond;
wherein X_{G} comprises a residue selected from the group consisting of F, I, a hydrophobic amino acid or an aromatic amino acid; and
wherein X_{H} comprises a residue selected from the group consisting of K or other amino acid capable of covalently bonding with another chemical entity;
homologs, isomers and active fragments thereof.

2. A composition comprising one or more peptides,
wherein the peptides comprise 13 to about 20 amino acid residues, and wherein the amino acid sequence of the residues comprises:
X₁X₂X₃X₄X₅X₆X₇X₈X₉X₁₀X₁₁X₁₂X₁₃
wherein X₁ comprises a residue selected from the group consisting of any—amino acids;
wherein X₂ comprises a residue selected from the group consisting of basic amino acids and A;
wherein X₃ comprises a residue selected from the group consisting of a basic amino acids and A;
wherein X₄ comprises a residue selected from the group consisting of a basic amino acids and A;
wherein X₅ comprises a residue selected from the group consisting of any amino acid;
wherein X₆ comprises a residue selected from the group consisting of A, Y, Q, W, V, T, P, N, M, I, F, G, and E;
wherein X₇ comprises a residue selected from the group consisting of basic amino acids and A;
wherein X₈ comprises a residue selected from the group consisting of V or a hydrophobic amino acids;
wherein X₉ comprises a residue selected from the group consisting of basic amino acids and A;
wherein X₁₀ comprises a residue selected from the group consisting of I, V, L, and hydrophobic amino acids;
wherein X₁₁ comprises a residue selected from the group consisting of I, V, V, F, and hydrophobic amino acids;
wherein X₁₂ comprises a residue selected from the group consisting of F, I, and hydrophobic amino acids;
wherein X₁₃ comprises a residue selected from the group consisting of K or other amino acid capable of covalently bonding with another chemical entity,
homologs, isomers and active-fragments thereof.

3. The composition of claim 2,
wherein X₁ comprises a residue selected from the group consisting of A, D, E, F, G, H, I, K, L, M, N, P, Q, R, S, T, V, W and Y;
wherein X₂ comprises a residue selected from the group consisting of basic amino acids, A, H, K, Q, R, S, and V;
wherein X₃ comprises a residue selected from the group consisting of basic amino acids, A, E, G, H, I, K, L, N, Q, R, S, V, and Y;
wherein X₄ comprises a residue selected from the group consisting of basic amino acids, A, E, H, I, K, L, N, P, Q, R, S, T, V, and Y;
wherein X₅ comprises a residue selected from the group consisting of A, D, E, F, G, H, K, L, M, N, P, Q, R, S, T, V, W and Y;
wherein X₆ comprises a residue selected from the group consisting of A, Y, Q, W, V, T, P, N, M, I, F, G, K, L, R, S and E;
wherein X₇ comprises a residue selected from the group consisting of basic amino acids, A, E, F, G, H, 1, K, M, N, P, Q, R, S, T, V, W, and Y;
wherein X₈ comprises a residue selected from the group consisting of hydrophobic amino acids, A, F, G, I, K, P, Q, R, S, V, W, and Y;
wherein X₉ comprises a residue selected from the group consisting of basic amino acids, A, E, F, G, H, I, K, L, M, N, P, Q, R, S, T, V, W, and Y;
wherein X₁₀ comprises a residue selected from the group consisting of hydrophobic amino acids, 1, V, L, A, E, F, G, H, K, N, P, Q, R, S, T, W, Y, Aib, and Cha, 3,3-diphenylAla, homoPhe, and phenylGly;
wherein X₁₁ comprises a residue selected from the group consisting of hydrophobic amino acids, I, V, F, A, D, E, G, K, M, N, P, Q, R, S, T, W, Y, Ahx, Nal, phenylGly, Cha, homoPhe, 3,4-dichloroPhe, 3,4-diphenylPhe, 4-chloroPhe, and 4-nitroPhe;
wherein X₁₂ comprises a residue selected from the group consisting of hydrophobic amino acids, F, I, A, E, G, H, K, M, N, P, R, S, T, V, W, Y, Ahx, Cha, homoPhe, phenylGly, and 3,3-diphenylAla;
wherein X₁₃ comprises K.

4. The composition of claim 2, wherein:
X₁ comprises a residue selected from the group consisting of any amino acid;
wherein X₂ comprises a residue selected from the group consisting of a basic amino acids and A;
wherein X₃ comprises a residue selected from the group consisting of a basic amino acids and A;
wherein X₄ comprises a residue selected from the group consisting of basic amino acids and A;
wherein X₅ comprises a residue selected from the group consisting of any amino acid;
wherein X₆ comprises a residue selected from the group consisting of A, Y, Q, W, V, T, P, N, M, I, F, G, and E;
wherein X₇ comprises a residue selected from the group consisting of basic amino acids and A;
wherein X₈ comprises a residue selected from the group consisting of hydrophobic amino acids and V;
wherein X₉ comprises a residue selected from the group consisting of basic amino acids and A;
wherein X₁₀ comprises a residue selected from the group consisting of hydrophobic amino acids, I, V, and L;
wherein X₁₁ comprises a residue selected from the group consisting of hydrophobic amino acids, I, V, L, and F;
wherein X₁₂ comprises a residue selected from the group consisting of hydrophobic amino acids, F, and I;
and wherein X₁₃ comprises K.

5. The composition of claim 3,
wherein X₁ comprises a residue selected from the group consisting of A and R;
wherein X₂ comprises R;
wherein X₃ comprises R;
wherein X₄ comprises R;
wherein X₅ comprises R;
wherein X₆ comprises Q;
wherein X₇ comprises a residue selected from the group consisting of A and R;
wherein X₈ comprises V;
wherein X₉ comprises a residue selected from the group consisting of A and R;
wherein X₁₀ comprises I;
wherein X₁₁ comprises a residue selected from the group consisting of I, d-Isoleucine and Y;
wherein X₁₂ comprises F;
wherein X₁₃ comprises K;
further comprising a linker,
wherein the linker comprises goalpost 3, iminodiacetic acid, or lysine.

6. The composition of claim 2, wherein the peptide is selected from one of the following: SEQ ID NO:132; SEQ ID NO:167; SEQ ID NO:228; SEQ ID NO:254; SEQ ID NO:267; SEQ ID NO:279; SEQ ID NO:318; SEQ ID NO:331; SEQ ID NO:375; SEQ ID NO:376; and SEQ ID NO:381.

7. The composition of claim 1, wherein the peptide comprises an amino acid sequence as set forth in SEQ ID NOS. 1-524, and active fragments thereof.

8. The composition of claim 1, wherein the composition comprises dimers, homodimers, heterodimers, or multimers.

9. The composition of claim 8,
wherein the peptides are linked at covalent bonding sites, wherein the covalent bonding sites comprise C-termini, N-termini, functional groups, lysine, protected lysine; or,
wherein the peptides are attached to an optional linker; or
wherein the peptides are attached to one or more spacer molecules.

10. The composition of claim 9, wherein the linker is selected from the group consisting of:

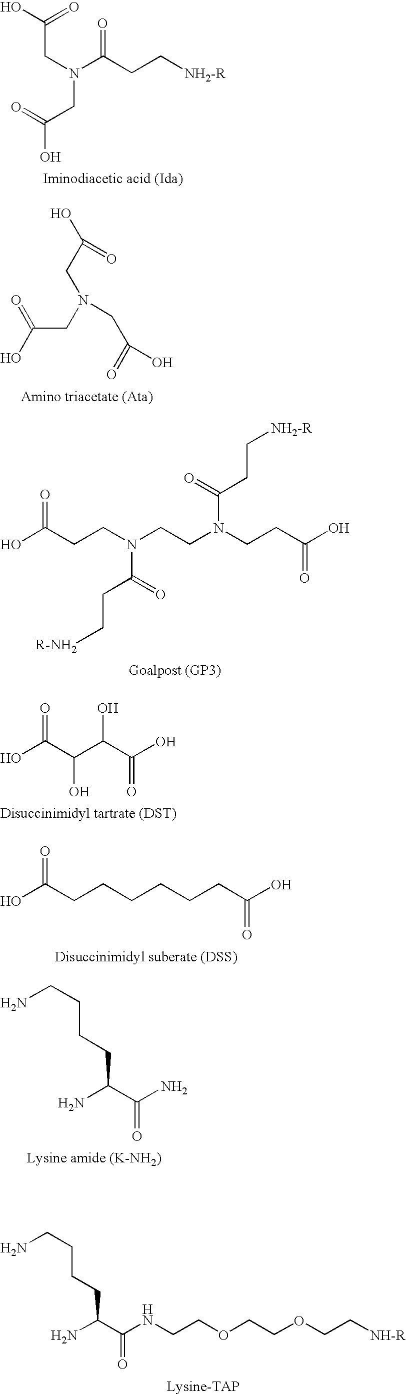

11. The composition of claim 10, wherein the linker comprises; 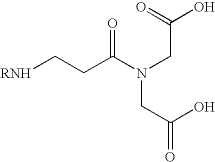
wherein R is H, Boc, or PEG and the epsilon amine of lysine in position 13 is bound to the linker with amide bonds.

12. The composition of claim 11, wherein R═H.

13. The composition of claim 9, wherein the linking agent comprises lysine.

14. The composition of claim 1, wherein one or more peptides is modified by the presence of a water soluble moiety, methylation, acetylation, addition of a benzyloxycarbonyl group, addition of a blocking group, incorporation of a desamino acid, addition of a carboxamide group, formation of a cyclic lactam, cyclization, addition of side chains, or phosphorylation.

15. The composition of claim 14, wherein the water soluble moiety comprises polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl-pyrrolidone)polyethylene glycol, propropylene glycol homopolymers, polypropylene oxide/ethylene oxide copolymers, or polyoxyethylated polyols.

16. A method for inhibiting cellular proliferation comprising administering a composition comprising one or more peptides;
wherein the peptides comprise 8-40 amino acid residues in length, and the amino acid residue sequence of the residues comprises:
X₁—(X_{A})_{N}—X_{B}—X_{C}—(X_{A})_{N}—X_{D}—(X_{A})_{N}—X_{E}—X_{F}—X_{G}—X_{H},
wherein X₁ comprises a residue selected from the group consisting of any amino acid;
wherein X_{A} comprises a number (N) of residues individually selected from the group consisting of basic amino acids and A;
wherein N is an integer selected from the group consisting of 1, 2, 3, 4, 5, 6, 7, 8, 9 and 10;
wherein X_{B} comprises a residue selected from the group consisting of any amino acid;
wherein X_{C} comprises a residue selected from the group consisting of A, Y, Q, W, V, T, P, N, M, I, F, G, and E;
wherein X_{D} comprises a residue selected from the group consisting of V, a hydrophobic amino acid, or a valence bond;
wherein X_{E} comprises a residue selected from the group consisting of I, V, L, a hydrophobic amino acid or a valence bond;
wherein X_{F} comprises a residue selected from the group consisting of I, V, L, F, a hydrophobic amino acid, an aromatic amino acid or a valence bond;
wherein X_{G} comprises a residue selected from the group consisting of F, I, a hydrophobic amino acid or an aromatic amino acid; and
wherein X_{H} comprises a residue selected from the group consisting of K or other amino acid capable of covalently bonding with another chemical entity;
homologs, isomers and active fragments thereof.

17. A method for inhibiting cellular proliferation comprising administering a composition comprising one or more peptides;
wherein the peptides comprise 13 to about 20 amino acid residues, and wherein the amino acid sequence of the residues comprises:
X₁X₂X₃X₄X₅X₆X₇X⁸X₉X₁₀X₁₁X₁₂X₁₃
wherein X₁ comprises a residue selected from the group consisting of any amino acids;
wherein X₂ comprises a residue selected from the group consisting of basic amino acids and A;
wherein X₃ comprises a residue selected from the group consisting of a basic amino acids and A;
wherein X₄ comprises a residue selected from the group consisting of a basic amino acids and A;
wherein X₅ comprises a residue selected from the group consisting of any amino acid;
wherein X₆ comprises a residue selected from the group consisting of A, Y, Q, W, V, T, P, N, M, I, F, G, and E;
wherein X₇ comprises a residue selected from the group consisting of basic amino acids and A;
wherein X₈ comprises a residue selected from the group consisting of V or a hydrophobic amino acids;
wherein X₉ comprises a residue selected from the group consisting of basic amino acids and A;
wherein X₁₀ comprises a residue selected from the group consisting of I, V, L, and hydrophobic amino acids;
wherein X₁₁ comprises a residue selected from the group consisting of I, V, V, F, and hydrophobic amino acids;
wherein X₁₂ comprises a residue selected from the group consisting of F, I, and hydrophobic amino acids;
wherein X₁₃ comprises a residue selected from the group consisting of K or other amino acid capable of covalently bonding with another chemical entity,
homologs, isomers and active-fragments thereof.

18. The method of claim 17 wherein:
wherein X₁ comprises a residue selected from the group consisting of A, D, E, F, G, H, I, K, L, M, N, P, Q, R, S, T, V, W and Y;
wherein X₂ comprises a residue selected from the group consisting of basic amino acids, A, H, K, Q, R, S, and V;
wherein X₃ comprises a residue selected from the group consisting of basic amino acids, A, E, G, H, I, K, L, N, Q, R, S, V, and Y;
wherein X₄ comprises a residue selected from the group consisting of basic amino acids, A, E, H, I, K, L, N, P, Q, R, S, T, V, and Y;
wherein X₅ comprises a residue selected from the group consisting of A, D, E, F, G, H, K, L, M, N, P, Q, R, S, T, V, W and Y;
wherein X₆ comprises a residue selected from the group consisting of A, Y, Q, W, V, T, P, N, M, I, F, G, K, L, R, S and E;
wherein X₇ comprises a residue selected from the group consisting of basic amino acids, A, E, F, G, H, I, K, M, N, P, Q, R, S, T, V, W, and Y;
wherein X₈ comprises a residue selected from the group consisting of hydrophobic amino acids, A, F, G, I, K, P, Q, R, S, V, W, and Y;
wherein X₉ comprises a residue selected from the group consisting of basic amino acids, A, E, F, G, H, I, K, L, M, N, P, Q, R, S, T, V, W, and Y;
wherein X₁₀ comprises a residue selected from the group consisting of hydrophobic amino acids, I, V, L, A, E, F, G, H, K, N, P, Q, R, S, T, W, Y, Aib, and Cha, 3,3-diphenylAla, homoPhe, and phenylGly;
wherein X₁₁ comprises a residue selected from the group consisting of hydrophobic amino acids, I, V, F, A, D, E, G, K, M, N, P, Q, R, S, T, W, Y, Ahx, Nal, phenylGly, Cha, homoPhe, 3,4-dichloroPhe, 3,4-diphenylPhe, 4-chloroPhe, and 4-nitroPhe;
wherein X₁₂ comprises a residue selected from the group consisting of hydrophobic amino acids, F, I, A, E, G, H, K, M, N, P, R, S, T, V, W, Y, Ahx, Cha, homoPhe, phenylGly, and 3,3-diphenylAla;
wherein X₁₃ comprises K.

19. The method of claim 17,
wherein X₁ comprises a residue selected from the group consisting of any amino acid;
wherein X₂ comprises a residue selected from the group consisting of a basic amino acids and A;
wherein X₃ comprises a residue selected from the group consisting of a basic amino acids and A;
wherein X₄ comprises a residue selected from the group consisting of basic amino acids and A;
wherein X₅ comprises a residue selected from the group consisting of any amino acid;
wherein X₆ comprises a residue selected from the group consisting of A, Y, Q, W, V, T, P, N, M, I, F, G, and E;
wherein X₇ comprises a residue selected from the group consisting of basic amino acids and A;
wherein X₈ comprises a residue selected from the group consisting of hydrophobic amino acids and V;
wherein X₉ comprises a residue selected from the group consisting of basic amino acids and A;
wherein X₁₀ comprises a residue selected from the group consisting of hydrophobic amino acids, I, V, and L;
wherein X₁₁ comprises a residue selected from the group consisting of hydrophobic amino acids, I, V, L, and F;
wherein X₁₂ comprises a residue selected from the group consisting of hydrophobic amino acids, F, and I;
and wherein X₁₃ comprises K.

20. The method of claim 18,
wherein X₁ comprises a residue selected from the group consisting of A and R;
wherein X₂ comprises R;
wherein X₃ comprises R;
wherein X₄ comprises R;
wherein X₅ comprises R;
wherein X₆ comprises Q;
wherein X₇ comprises a residue selected from the group consisting of A and R;
wherein X₈ comprises V;
wherein X₉ comprises a residue selected from the group consisting of A and R;
wherein X₁₀ comprises 1;
wherein X₁₁ comprises a residue selected from the group consisting of I, d-Isoleucine and Y;
wherein X₁₂ comprises F;
wherein X₁₃ comprises K;
further comprising a linker,
wherein the linker comprises goalpost 3, iminodiacetic acid, or lysine.

21. The method of claim 17, wherein the peptide is selected from one of the following: SEQ ID NO:132; SEQ ID NO:167; SEQ ID NO:228; SEQ ID NO:254; SEQ ID NO:267; SEQ ID NO:279; SEQ ID NO:318; SEQ ID NO:331; SEQ ID NO:375; SEQ ID NO:376; and SEQ ID NO:381.

22. The method of claim 17, wherein the peptide comprises an amino acid sequence as set forth in SEQ ID NOS. 1-524 and active fragments thereof.

23. The method of claim 17, wherein the composition comprises dimers, homodimers, heterodimers, or multimers.

24. The method of claim 17, wherein the peptides are linked at covalent bonding sites, wherein the covalent bonding sites comprise C-termini, N-termini, functional groups, lysine, or protected lysine.

25. The method of claim 17, wherein the linker is selected from the group consisting of:

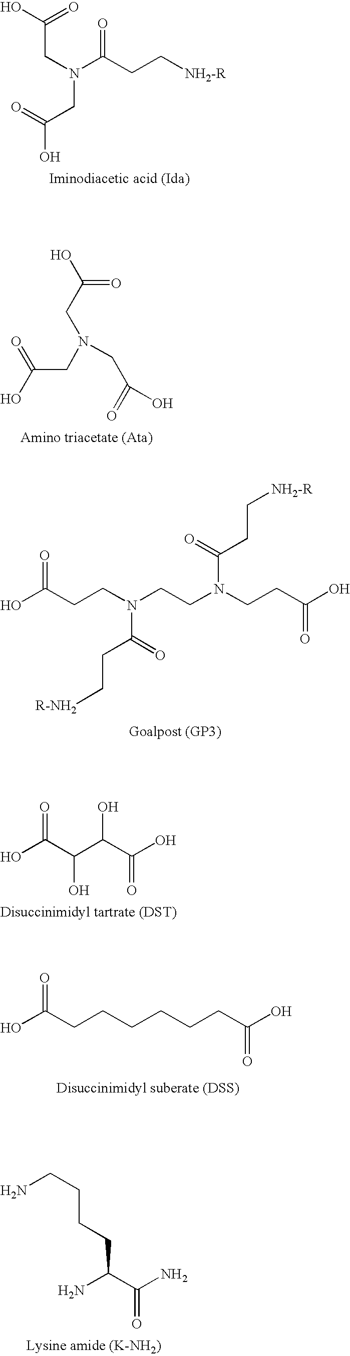

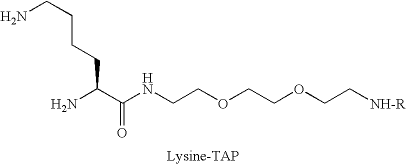

26. The method of claim 17, wherein the linking agent comprises: 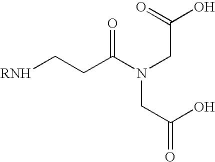
wherein R is H, Boc, or PEG and the epsilon amine of lysine in position 13 is bound to the linker with amide bonds.

27. The method of claim 25, wherein R═H.

28. The method of claim 24, wherein the linking agent comprises lysine.

29. The method of claim 16, wherein one or more peptides is modified by the presence of a water soluble moiety, methylation, acetylation, addition of a benzyloxycarbonyl group, addition of a blocking group, incorporation of a desamino acid, addition of a carboxamide group, formation of a cyclic lactam, cyclization, addition of side chains, or phosphorylation.

30. The method of claim 25, wherein the water soluble moiety comprises polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl-pyrrolidone)polyethylene glycol, propropylene glycol homopolymers, polypropylene oxide/ethylene oxide copolymers, or polyoxyethylated polyols.

31. The method of claim 16, wherein the cellular proliferation comprises undesirable cellular proliferation.

32. The method of claim 26, wherein the undesirable cellular proliferation is associated with atherosclerosis, solid tumors; blood-borne tumors, such as leukemias; tumor metastasis; benign tumors, hemangiomas, acoustic neuromas, neurofibromas, trachomas, pyogenic granulomas, vascular malfunctions, abnormal wound healing, inflammatory, immune disorders, Behcet's disease, gout, gouty arthritis, abnormal angiogenesis accompanying rheumatoid arthritis, skin diseases, psoriasis, diabetic retinopathy, ocular angiogenic diseases, retinopathy of prematurity, retrolental fibroplasia, macular degeneration, corneal graft rejection, neovascular glaucoma, liver diseases or Oster Webber Syndrome (Osler-Weber-Rendu disease).
